(19) United States
(12) Patent Application Publication
(10) Pub. No.: US 2017/0173114 A1
(43) Pub. Date: Jun. 22, 2017

(54) METHODS AND COMPOSITIONS FOR INDUCTION OF UCP1 EXPRESSION

(71) Applicant: Joslin Diabetes Center, Inc., Boston, MA (US)

(72) Inventors: C. Ronald Kahn, West Newton, MA (US); Yu-Hua Tseng, Newton, MA (US)

(21) Appl. No.: 15/309,320

(22) PCT Filed: May 7, 2015
(86) PCT No.: PCT/US15/29744

§ 371 Date: Nov. 7, 2016

Related U.S. Application Data
(60) Provisional application No. 61/989,628, filed on May 7, 2014.

(51) Int. Cl. A61K 38/18; A61K 35/28; A61K 48/00; A61K 39/395; A61K 35/12

(52) U.S. Cl. CPC A61K 38/1825; A61K 39/39541; A61K 35/12; A61K 48/005; A61K 35/28; A61K 2121/00

(57) ABSTRACT

The present invention provides methods and compositions for the induction of expression of UCP1 independent of lipid accumulation. The invention, in particular, features methods for converting FGF receptive cells, e.g., preadipocyte cells, into energy consuming cells through FGF-mediated UCP1 expression. The invention further provides methods and compositions for treating metabolic disorders with an FGF receptor agonist, (e.g., an FGF protein, or fragment thereof, a nucleic acid encoding an FGF protein, an FGF mimetic, an anti-FGF receptor agonist antibody, or antigen binding fragment thereof), or a cell contacted with an FGF receptor agonist, including FGF6.

### RELATED APPLICATIONS

This application claims the benefit of priority to U.S. Provisional Patent Appln. No. 61/989,628, filed on May 7, 2014. The entire contents of the aforementioned priority application are incorporated by reference herein.

### GOVERNMENT RIGHTS

This invention was made with U.S. government support under grant number NIDDK R01 DK077097 awarded by the National Institutes of Health. The government has certain rights in the invention.

### BACKGROUND OF THE INVENTION

Metabolic disorders represent a major risk factor for several common medical conditions, including obesity, diabetes mellitus, dyslipidemia, non-alcoholic fatty liver disease, cardiovascular disease, and certain cancers. As such, novel therapies for treating obesity and related metabolic conditions such as diabetes are of the utmost importance for healthcare and research communities.

In mammals, there are two functionally different types of fat: white adipose tissue (WAT), the primary site of triglyceride storage, and brown adipose tissue (BAT), which is specialized in thermogenic energy expenditure (Cannon B. et al., Physiol. Rev. 84:277-359, 2004). Brown adipose tissue plays a pivotal role in adaptive thermogenesis, a physiological process during which energy is dissipated in response to environmental changes, such as cold and diet (Lowell B. B. et al., Nature 404:652-660, 2000; Tseng Y. H., et al., Nat. Rev. Drug Discov. 9:465-482, 2010). Two developmentally-distinct types of brown adipocytes exist in mammals: the classical or constitutive BAT (cBAT) that arises during embryogenesis (Seale P. et al., Nature 454:961-967, 2008); and the inducible or recruitable BAT (rBAT), also known as the beige or brite adipocytes, (Petrovic N. et al., J. Biol. Chem. 285:7153-7164, 2010; Enerback S., et al., N. Engl. J. Med. 360:2021-2023, 2009; Ishibashi J. et al., Science 328:1113-1114, 2010) that is recruited postnatally within WAT or skeletal muscle (Guerra C. et al., J. Clin. Invest. 102:412-420, 1998; Almind K. et al., Proc. Natl. Acad. Sci. USA 104:2366-2371, 2007). An important cross-talk has recently been demonstrated between these two types of brown adipose tissue (Schulz T. J. et al., Nature 495:379-383, 2013). When impaired, cBAT is able to signal through the sympathetic nervous system to induce the formation of rBAT within subcutaneous WAT. This previously unknown compensatory mechanism, aimed at restoring total brown fat-mediated thermogenic capacity in the body, is sufficient to maintain normal temperature homeostasis and resistance to diet-induced obesity.

Heat is generated directly by protons rushing down their electrochemical gradient and also indirectly by the subsequent increase in flux through the electron transport chain that follows. This process is also known as thermogenesis (Cannon B. et al., Int. J. Obes. (Lond) 34 Suppl 1:S7-16, 2010). UCP1 is unique to brown adipose tissue, can serve as a defining marker of brown adipocytes, and is necessary to mediate brown adipose tissue thermogenesis (Golozoubova V. et al., FASEB J. 15:2048-2050, 2001). While other tissues possess different members of the UCP family, UCP1 is the only carrier that can promote heat production (Nautiyal J. et al., Trends Endocrinol Metab 24:451-459, 2013). Thus, UCP1-deficient mice are cold sensitive (Enerback S. et al., Nature 387:90-94, 1997) and exhibit increased susceptibility to diet-induced obesity (Lowell B. B. et al., Nature 366:740-742, 1993; Kontani Y. et al., Aging Cell 4:147-155, 2005; Feldmann H. M. et al., Cell. Metab. 9:203-209, 2009). Conversely, transgenic mice with UCP1 expression in white fat display lean phenotype (Kopecky J. et al., J. Gin. Invest. 96:2914-2923, 1995; Leonardsson G., et al., Proc. Natl. Acad. Sci. USA 101:8437-8442, 2004).

BAT is specialized to dissipate chemical energy in the form of heat and has recently been shown to be present in humans (Nedergaard J. et al., Am. J. Physiol. Endocrinol. Metab. 293:E444-E452, 2007; Cypess A. M. et al., N. Engl. J. Med. 360:1509-1517, 2009; Marken Lichtenbelt W. D. et al., N. Engl. J. Med. 360:1500-1508, 2009; Saito M. et al., Diabetes 58:1526-1531, 2009; Virtanen K. A. et al., N. Engl. J. Med. 360:1518-1525, 2009; Zingaretti M. C. et al., FASEB J. 23:3113-3120, 2009; Celi F. S. et al., N. Engl. J. Med. 360:1553-1556, 2009). BAT dissipates energy as heat to maintain optimal thermogenesis. The energetic processes executed by BAT require a readily available fuel supply, which includes glucose and lipids. Indeed, studies indicate that BAT is involved in triglyceride clearance and glucose disposal (Bartelt A. et al., Nat. Med. 17:200-205, 2011; Williams K. J. et al., Nat. Med. 17:157-159, 2011; Nedergaard J. et al., Cell. Metab. 13:238-240, 2011). Lipids become available by cellular uptake, de novo lipogenesis, and from release of fat stored in the multilocular lipid droplets of brown adipocytes, a process called lipolysis. BAT also possesses a great capacity for glucose uptake and metabolism, as well as an ability to modulate insulin sensitivity (Schulz T. J. et al., Biochem J 453:167-178, 2013) making BAT a target for the treatment of metabolic disorders.

Given BAT's immense capacity for energy expenditure (Cannon B. et al., Physiol. Rev. 84:277-359, 2004) and newly identified effects on fatty acid and glucose metabolism (Bartelt A. et al., J. Mol. Med. (Berl) 2012), the ability to increase the amount and activity of BAT is of interest as a possible therapy for treating diseases such as obesity and diabetes.

The unique property of BAT to be able to mediate energy expenditure and thermogenesis is dependent on the presence of uncoupling protein 1 (UCP1), whose expression is specific to BAT. While there are more than forty members of the mitochondrial carrier family, UCP1 is the only carrier able to permit proton translocation across the mitochondrial inner membrane. During this process, UCP1 robustly facilitates fatty acid oxidation and dissipates energy as heat while uncoupling respiration from ATP synthesis. Ectopic overexpression of UCP1 in non-adipocytes results in enhanced mitochondrial uncoupling and increased energy expenditure (Casteilla L. et al., Proc. Nat.l Acad. Sci. USA 87:5124-5128, 1990; Gonzalez-Muniesa P. et al., J. Physiol. Biochem. 61:389-393, 2005; Li, B. et al., Nat. Med. 6:1115-1120, 2000). Studies reveal that forced expression of UCP1 in Chinese hamster ovary (CHO) cells (Casteilla L. et al., Proc. Nat.l Acad. Sci. USA 87:5124-5128, 1990) or HepG2 hepatocyte cell lines (Gonzalez-Muniesa P. et al., J. Physiol. Biochem. 61:389-393, 2005) is sufficient to induce uncoupling of respiration and decrease ATP production. Transgenic mice expressing UCP1 in skeletal muscle are protected from high fat diet-induced obesity and display enhanced glucose uptake in skeletal muscle and improved insulin sensitivity (Li, B. et al., Nat. Med. 6:1115-1120, 2000). These findings indicate that UCP1 can function alone in non-adipocytes. Therefore, the up regulation of UCP1 in white adipose tissue or even non-adipocytes could mimic the function of brown adipose tissue and promote metabolic health. However, no factor has been identified that is able to induce UCP1 expression independent of brown/beige adipocyte differentiation.

Given the essential role of UCP1 in brown fat-mediated thermogenesis, molecules that are able to promote UCP1 expression and function provide avenues for the development of new therapies to treat obesity and other metabolic conditions. Accordingly, there is a need in the art for methods for the regulation of UCP1 expression, mitochondrial function and energy metabolism as therapies for obesity or diabetes and related disorders.

### SUMMARY OF INVENTION

The present invention is based, at least in part, on the novel finding that certain fibroblast growth factors, e.g., FGF2, FGF6, FGF9, can induce uncoupling protein 1 (UCP1) in a cell in a manner that is independent of cell differentiation, as UCP1 can function independent of brown adipocyte differentiation. Thus, the invention includes in one embodiment methods and compositions for upregulating UCP1 (e.g., by administration of FGF6) in white adipose tissue (WAT) or non-adipocyte cells in order to increase energy consumption. Such energy consumption—usually attributed to BAT but determined herein to be possible in preadipocytes and WAT—can be used therapeutically to treat metabolic disorders, such as obesity, diabetes, or metabolic syndrome. In addition, in a further embodiment, the invention includes methods and compositions relating to increasing energy consumption in mature adipocytes, through treatment or exposure to an FGF, e.g., FGF6.

In one particular embodiment, the present invention provides methods and compositions relating to the use of FGF receptor agonists for inducing UCP1 expression in FGF-receptive cells, whereby the UCP1 expression results in the ability of the cell to consume energy in the absence of differentiation.

One aspect of the invention provides methods of expressing uncoupling protein 1 (UCP1) in an FGF-receptive cell, the method comprising contacting the FGF-receptive cell with an FGF receptor agonist, in an amount sufficient to induce UCP1 expression, such that UCP1 is expressed in the FGF-receptive cell, wherein the FGF-receptive cell does not exhibit substantial lipid accumulation following contact with the FGF receptor agonist, e.g., FGF protein or nucleic acid encoding the FGF protein.

In another aspect, the invention provides methods of expressing uncoupling protein 1 (UCP1) in an FGF-receptive cell, the method comprising contacting the FGF-receptive cell with an FGF receptor agonist, in an amount sufficient to induce UCP1 expression, such that UCP1 is expressed in the FGF-receptive cell, wherein the FGF-receptive cell is a preadipocyte and does not differentiate into a brown adipocyte following contact with the FGF receptor agonist.

In one embodiment of the invention, the FGF-receptive cell is an undifferentiated cell. In another embodiment of the invention, the undifferentiated cell is selected from the group consisting of a primary adipose precursor, an adult stem cell, an embryonic stem cell, an induced pluripotent stem cell, a stromal-vascular fraction cell, an immortalized human brown fat precursor cell, an immortalized human white fat precursor cell, a brown preadipocyte, and a white preadipocyte.

In a further embodiment of the invention, the FGF-receptive cell is contacted with the FGF receptor agonist in vitro. In another embodiment, the FGF-receptive cell is contacted with the FGF receptor agonist in vivo. In yet a further embodiment, the method comprises implanting the FGF-receptive cell in a subject. In one embodiment, the subject has a disorder that would benefit from metabolic control. In a particular embodiment, the subject is human. In one embodiment, the disorder that would benefit from metabolic control is selected from the group consisting of a disorder that would benefit from glucose control, a disorder that would benefit from weight control, a disorder that would benefit from cholesterol control, and a fatty acid metabolism disorder. In a particular disorder, the disorder that would benefit from glucose control is selected from the group consisting of insulin resistance, diabetes, and hyperglycemia. In another embodiment, the disorder that would benefit from weight control is selected from the group consisting of liver disease, dyslipidemia, a glycemic control disorder, cardiovascular disease and obesity. In yet another embodiment, the disorder that would benefit from cholesterol control is heart disease. In a particular embodiment, the disorder is metabolic syndrome. In yet another embodiment, the subject has insulin resistance and/or insulin insensitivity.

In another embodiment, the FGF-receptive cell does not exhibit substantial increases in expression of a brown adipocyte marker selected from the group consisting of PR Domain Containing 16 (PRDM16), PPAR-gamma Coactivator 1 (PGC1), Adipocyte Protein 2 (Ap2), and Cell Death Inducing DFFA-Like Effector A (CIDEA).

Another aspect of the invention provides methods of treating a subject having a disorder that would benefit from metabolic control, the method comprising administering a composition comprising an FGF receptor agonist to the subject, such that the disorder is treated, wherein the FGF receptor agonist is administered to the subject in the absence of an additional agent selected from the group consisting of an additional growth factor, dexamethasone, and indomethacin.

In one embodiment of the invention, the FGF receptor agonist is administered to the subject by injection. In another embodiment, the injection is subcutaneous.

In yet another embodiment, the FGF receptor agonist is a nucleic acid encoding an FGF protein and is administered to the subject via a viral vector. In a further embodiment, the FGF receptor agonist is administered to the subject via a drug delivery matrix. In one embodiment, the drug delivery matrix is silk hydrogel. In one embodiment, the FGF receptor agonist is administered to adipose tissue of the subject.

One aspect of the invention provides ex vivo methods of treating a subject having a disorder that would benefit from metabolic control, the method comprising administering an FGF-receptive cell contacted with an FGF receptor agonist to the subject, such that the disorder is treated, wherein the FGF-receptive cell is administered to the subject in the absence of an additional agent selected from the group consisting of an additional growth factor, dexamethasone, and indomethacin.

In one embodiment, the disorder is selected from the group consisting of a disease that would benefit from glucose control, a disease that would benefit from weight control, a disease that would benefit from cholesterol control, and a fatty acid metabolism disorder. In one embodiment the disease that would benefit from glucose control is selected from the group consisting of insulin resistance, diabetes, and hyperglycemia. In another embodiment, the disease that would benefit from weight control is selected from the group consisting of liver disease, dyslipidemia, a glycemic control disorder, cardiovascular disease and obesity.

In a further embodiment, the disease that would benefit from cholesterol control is heart disease. In a particular embodiment, the disorder is metabolic syndrome. In yet another embodiment, the subject has insulin resistance and/or insulin insensitivity.

In one embodiment, the subject is a human subject.

In one embodiment of the invention, the FGF receptor agonist is selected from the group consisting of an FGF protein (or functional fragment thereof), a nucleic acid encoding an FGF protein (or functional fragment thereof), an FGF mimetic, and an anti-FGF receptor agonist antibody, or an antigen-binding fragment thereof. In a particular embodiment, the FGF protein is not FGF21. In another embodiment, the FGF protein is selected from the group consisting of FGF1, FGF2, FGF4, FGF6, FGF8, FGF9, FGF16, FGF17, FGF18, and FGF20. In a particular embodiment, the FGF protein is FGF6.

In one embodiment the FGF receptor agonist is administered at a dose of about 0.5 mg/kg to about 300 mg/kg.

One aspect of the invention provides a method of treating a subject having diabetes or obesity, the method comprising administering a composition comprising an FGF6 protein or a nucleic acid encoding an FGF6 protein to the subject, such that the diabetes or obesity in the subject is treated, wherein the FGF6 protein or the nucleic acid encoding the FGF6 protein is administered to the subject in the absence an additional agent selected from the group consisting of an additional growth factor, dexamethasone, and indomethacin.

A further aspect of the invention provides an ex vivo method of treating a subject having obesity or diabetes, the method comprising administering an FGF-receptive cell contacted with an FGF6 protein or a nucleic acid encoding an FGF protein to the subject, such that obesity or diabetes in the subject is treated, wherein the FGF-receptive cell is administered to the subject in the absence of an additional agent selected from the group consisting of an additional growth factor, dexamethasone, and indomethacin.

In another aspect, the invention provides a method of treating metabolic syndrome in a subject, the method comprising selecting a subject having metabolic syndrome, and administering FGF6 protein or a nucleic acid encoding an FGF6 protein to the subject, such that the metabolic syndrome in the subject is treated.

A further aspect of the invention provides an ex vivo method of treating metabolic syndrome in a subject, the method comprising selecting a subject having metabolic syndrome, and administering an FGF-receptive cell contacted with FGF6 protein or a nucleic acid encoding an FGF6 protein to the subject, such that the metabolic syndrome in the subject is treated.

In one embodiment, the FGF-receptive cell is administered to the subject in the absence of an additional agent selected from the group consisting of an additional growth factor, dexamethasone, and indomethacin. In another embodiment, the subject has or is at risk for insulin resistance and/or insulin insensitivity. In one embodiment, the FGF6 protein or the nucleic acid encoding the FGF6 protein is administered to the subject by injection. In another embodiment, the injection is subcutaneous. In yet another embodiment, the nucleic acid is administered to the subject via a viral vector. In one embodiment, the FGF6 protein or the nucleic acid encoding the FGF6 protein is administered to the subject via a drug delivery matrix. In another embodiment, the drug delivery matrix is silk hydrogel. In a further embodiment, the FGF6 protein, the nucleic acid encoding the FGF6 protein, or the FGF-receptive cell is administered to adipose tissue of the subject. In a particular embodiment, an anti-FGFR1 agonist antibody is administered to the subject. In another embodiment, the subject is human.

One aspect of the invention provides methods for lowering the weight of a subject, comprising selecting a subject in need of weight loss, and locally administering to white adipose tissue of the subject an FGF receptor agonist, thereby lowering the weight of the subject.

In one embodiment of the invention, the subject has a disorder selected from the group consisting of a disease that would benefit from glucose control, a disease that would benefit from weight control, a disease that would benefit from cholesterol control, and a fatty acid metabolism disorder. In another embodiment, the disease that would benefit from glucose control is selected from the group consisting of insulin resistance, diabetes, and hyperglycemia. In a further embodiment, the disease that would benefit from weight control is selected from the group consisting of liver disease, dyslipidemia, a glycemic control disorder, cardiovascular disease and obesity. In yet another embodiment, the disease that would benefit from cholesterol control is heart disease. In a particular embodiment, the disorder is metabolic syndrome. In another embodiment, the subject has insulin resistance and/or insulin insensitivity. In a particular embodiment, the subject is human.

In one embodiment of the invention, the FGF receptor agonist is selected from the group consisting of an FGF protein (or functional fragment thereof), a nucleic acid encoding an FGF protein (or functional fragment thereof), an FGF mimetic, and an anti-FGF receptor agonist antibody, or an antigen-binding fragment thereof. In another embodiment, the FGF protein is not FGF21. In a further embodiment, the FGF protein is selected from the group consisting of FGF1, FGF2, FGF4, FGF6, FGF8, FGF9, FGF16, FGF17, FGF18, and FGF20. In a particular embodiment, the FGF protein is FGF6. In a further embodiment, the FGF receptor agonist is administered subcutaneously to the subject.

In one embodiment, the methods of the invention are performed locally in a subject in need thereof, e.g., an obese human subject. For example, the methods described herein may be directed to tissue primarily comprising beige adipocytes, white adipocytes, or brown adipocytes.

Another aspect of the invention is a method of generating immortalized human fat progenitors. In one embodiment, the fat progenitor is a human brown fat progenitor. In another embodiment, the fat progenitor is a human white fat progenitor. The method includes obtaining primary stromal-vascular fraction (SVF) cells from a human subject, and infecting the SVF cells with a virus that expresses human telomere reverse transcriptase (hTERT), such that immortalized human fat progenitors are generated. In one embodiment, the SVF cells are infected with the hTERT expressing virus at about 80% confluence. In one embodiment, the SVF cells are infected with the hTERT expressing virus until the SVF cells reach about 90% confluence. In a further embodiment, the SVF cells are infected with the virus in the presence of polybrene. In yet a further embodiment, the immortalized cells are selected using hygromycin selection.

### BRIEF DESCRIPTION OF THE DRAWINGS

FIG. 1A depicts UCP1-mediated heat production in mitochondria. FIG. 1B depicts a high-throughput screen of secreted proteins that was designed to identify proteins capable of inducing UCP1 expression.

FIG. 2A graphically depicts relative expression of FGF6 in the muscle, brown adipose tissue and white adipose tissue in mice in response to treatment CL316,243, which is a compound mimicking beta-adrenergic activation. FIG. 2B graphically depicts relative expression of FGF6 in mature adipocytes and stromal vascular fraction cells (SVF). PBS=phosphate buffered saline control. CL=CL316,243. SQ=subcutaneous white adipose tissue. EPI=epididymal white adipose tissue. BAT=brown adipose tissue. MUS=skeletal muscle. FIG. 2C graphically depicts the relative expression of FGF6 induced by cold exposure (4° C., 7 days) and FIG. 2D graphically depicts the relative expression of FGF6 induced by exercise training (14 days).

FIGS. 3A-3D graphically depict results from the treatment of murine brown preadipocytes with FGF6, vehicle control (“control” or “C”), or induction media (“induction” or “I”). FIG. 3A graphically depicts mRNA expression of adipogenic markers PPARγ, ap2 and FAS. FIG. 3B depicts mRNA expression of brown fat markers UCP1, PRDM16, PGC1α and CIDEA. FIG. 3C provides results from a Western blot of UCP1 and β-tubulin protein levels in cells exposed to the vehicle control or FGF6. FIG. 3D depicts acidification of cell culture media due to increased mitochondrial metabolism and accumulation of lipid as demonstrated by increased staining with oil red O.

FIGS. 4A-4C graphically depict FGF6 induction of UCP1 expression in brown preadipocytes in a dose-dependent and time-regulated manner. Specifically, FIG. 4A depicts a dose-response curve of UCP1 expression by FGF6 at day 7. Numbers above curve indicate fold-increase relative to vehicle control. FIG. 4B depicts the fold-induction of UCP1 by vehicle control (“C”), FGF6 (“F6”) or FGF21 (“F21”) within 24 hours of treatment. FIG. 4C depicts a time-course of UCP1 expression induced by 200 ng/ml of FGF6, compared with cells differentiated in regular induction media. Numbers above curves indicate fold-induction relative to day 0. FIG. 4D depicts the effect of FGF6 on cell proliferation (measured by MTT assay) at 24 and 72 hours.

FIGS. 5A-5C depict constitutive overexpression of FGF6 in WT-1 brown preadipocytes. Specifically, FIG. 5A depicts marked increases in UCP1 expression over basal levels (i.e., control (“cont”)) induced by constitutive overexpression of FGF6 in brown preadipocytes. FIG. 5B depicts a profile of cellular respiration developed by utilizing well-characterized mitochondrial toxins including, oligomycin, an inhibitor of ATP synthase, which allows measurement of ATP turnover; an uncoupler, FCCP, was used to measure respiratory capacity; and a complex 1 inhibitor, rotenone, that prevents electron transfer activity and leaves only non-mitochondrial activity to be measured. FIG. 5C depicts the bioenergetic profile including basal respiration, ATP turnover, proton leak and respiratory capacity of FGF6 overexpressing brown preadipocytes versus control cells (“cont”).

FIGS. 6A-6C depict constitutive overexpression of FGF6 in 3T3-F442A white preadipocytes. Specifically, FIG. 6A depicts marked increases in UCP1 expression over basal levels (i.e., control (“cont”)) induced by constitutive overexpression of FGF6 in white preadipocytes. FIG. 6B depicts a profile of cellular respiration developed by utilizing well-characterized mitochondrial toxins including, oligomycin, an inhibitor of ATP synthase, which allows measurement of ATP turnover; an uncoupler, FCCP, was used to measure respiratory capacity; and a complex 1 inhibitor, rotenone, that prevents electron transfer activity and leaves only non-mitochondrial activity to be measured. FIG. 6C depicts the bioenergetics profile including basal respiration, ATP turnover, proton leak and respiratory capacity of FGF6 overexpressing white preadipocytes versus control cells (“cont”).

FIGS. 7A-7B depict the profile of mitochondrial respiration in WT-1 brown preadipocytes treated with 200 ng/mL FGF6 for 24 hours. Data were normalized to DNA content. FIG. 7C depicts the relative ratio of coupled and uncoupled respiration in brown preadipocytes treated with FGF6, versus control cells (“buffer”).

FIGS. 8A-8B depict mitochondrial DNA copy number and mitochondrial gene expression in WT-1 brown preadipocytes following treatment with FGF6 for 24 hours. FIG. 8A depicts that treatment of FGF6 does not alter mitochondrial DNA copy number in WT-1 brown preadipocytes. FIG. 8B depicts that the relative expression of nuclear-encoded mitochondrial genes was not altered upon treatment of FGF6. The left bars of FIG. 8B describe control cells, and the right bars describe FGF6-treated cells.

FIGS. 9A-9C depict FGF6 and FGF21 treatment of various cell types for three days. Specifically, FIG. 9A depicts FGF6 induction of UCP1 expression in primary stromo-vascular fraction (SVF) cells isolated from interscapular brown adipose tissue (BAT). FIG. 9B depicts FGF6 induction of UCP1 expression in primary stromo-vascular fraction cells (SVF) isolated from subcutaneous (SQ) white adipose tissue. FIG. 9C depicts no induction of UCP1 in C2C12 myogenic cells by treatment with FGF6 or FGF21. Treatment of cells with vehicle only is represented by the control (“cont”).

FIG. 10A depicts FGF6 induced expression of PTGS2 mRNA in brown preadipocytes following three days of treatment. FIG. 10B depicts FGF6 induced expression of COX2 protein in brown preadipocytes following three 3 days of treatment.

FIG. 11 depicts FGF6 induced UCP1 expression in brown preadipocytes is suppressed by NS-398, a selective COX2 inhibitor, in a dose dependent manner.

FIG. 12A depicts the loss of PTGS2 expression upon stably transfection of PTGS2-specific siRNA in DE cells. FIG. 12B depicts the loss of FGF6-mediated induction of UCP1 expression upon stable transfection of PTGS2-specific siRNA in DE cells.

FIG. 13A depicts FGF6 suppression of RIP140 expression in brown preadipocytes following 3 or 7 days of treatment with 200 ng/mL of FGF6 as compared to control (“cont”).

FIG. 13B depicts FGF6 suppression of RIP140 expression in white preadipocytes following 3 days of treatment with 200 ng/mL of FGF6 as compared to control (“cont”).

FIGS. 14A-14D depict UCP1 and PPARγ gene expression in murine brown preadipocyte WT-1 cells following treatment with FGF2, FGF6, FGF9, FGF21, BMP7 or vehicle (control) for 24 hours (FIG. 14A), 2 days (FIG. 14B), 5 days (FIG. 14C) and 7 days (FIG. 14D). All experiments were performed in triplicate and the data presented as mean+/−SEM.

FIG. 15 depicts UCP1 and PPARγ gene expression in murine brown preadipocyte WT-1 cells following treatment with FGF2, FGF6, FGF9, FGF21 or BMP7 for 24 hours, 2 days, 5 days and 7 days. All experiments were performed in triplicate and the data presented as mean+/−SEM.

FIG. 16 depicts UCP1 gene expression in murine brown preadipocyte WT-1 cells following treatment with FGF4, FGF22 or vehicle (control) for three days. All experiments were performed in triplicate and the data presented as mean+/−SEM.

FIGS. 17A and 17B depict UCP1 and PTGS2 gene expression in murine brown preadipocyte WT-1 cells following treatment with FGF4, FGF5, FGF6, FGF10, FGF16, FGF17, FGF18, FGF20 or buffer (control) for three days. All experiments were performed in triplicate and the data presented as mean+/−SEM.

FIG. 18 depicts UCP1 and PTGS2 gene expression in murine brown preadipocyte WT-1 cells following treatment with FGF1 (“F1”), FGF10 (“F10”) or vehicle control for three days. All experiments were performed in triplicate and the data presented as mean+/−SEM.

FIGS. 19A-19D depict UCP1 and PTGS2 gene expression in differentiated brown adipose cells and cells undergoing adipocyte differentiation. FIGS. 19A and 19B depict UCP1 and PTGS2 gene expression in WT-1 brown preadipocytes which were induced to become mature brown adipocytes by treatment with BMP7 in growth medium supplemented with insulin and triiodothyronine for 8 days. The differentiated cells were then treated with FGF6 or vehicle control (“ctl”) for 32 hours. FIGS. 19C and 19D depict UCP1 and PTGS2 gene expression in WT-1 brown preadipocytes which were induced to undergo differentiation by growth in growth medium supplemented with insulin and triiodothyronine for 3 days, followed by 48 hours of treatment in adipocyte induction media (growth medium supplemented insulin, T3, isobutyl-methylxanthine and dexamethasone). The cells were then treated with FGF2, FGF6, FGF9 or BMP7 in growth medium supplemented with insulin and triiodothyronine for two additional days. mRNA was isolated and subjected for Q-RT-PCR analysis for UCP1 and PTGS2. Experiments were performed in triplicate and the data presented as mean+/−SEM.

FIGS. 20A and 20B depict the effects of constitutive overexpression of FGF6 in WT-1 brown preadipocytes. Specifically, FIG. 20A depicts a profile of cellular glycolysis developed by utilizing well-characterized mitochondrial toxins including oligomycin, an inhibitor of ATP synthase, which shifts energy production to glycolysis, and a glucose analog, 2-DG, which allows the calculation of glycolytic reserve. FIG. 20B depicts the bioenergetic profile including glycolysis, glycolytic capacity, and glycolytic reserve of FGF6 overexpressing brown preadipocytes versus control cells (“entry”).

FIG. 21 depict the effects of constitutive overexpression of FGF6 on glucose uptake in preadipocytes. Specifically, FIG. 21 depicts induction of glucose uptake in FGF6 overexpressing WT-1 brown preadipocytes and F442A white preadipocytes versus control cells (“control” and “EGF”).

FIG. 22 graphically depicts results from the treatment of murine mature brown adipocytes with FGF6 or controls (“buffer” or BMP7). In particular, FIG. 22 graphically depicts mRNA expression of markers UCP1, PPARG2, PTGS2, NDST3 and SIRT1.

FIGS. 23A and 23B depict constitutive overexpression of FGF6 in differentiated WT-1 cells for 24 hours. FIG. 23A depicts a cellular respiration profile of FGF6 treated brown preadipocytes (upper line) versus control-treated cells (“buffer”; middle line) developed by utilizing well-characterized mitochondrial toxins including, oligomycin, an inhibitor of ATP synthase, which allows measurement of ATP turnover; an uncoupler, FGGP, was used to measure respiratory capacity; and a complex 1 inhibitor, rotenone, that prevents electron transfer activity and leaves only non-mitochondrial activity to be measured. A represents pyruvate; B represents 0.5 μM oligo; C represents 1 μM FCCP; and D represents 0.11 μM rotenone/2.2 μM ANT. FIG. 23B depicts induction of glucose uptake in FGF6 overexpressing WT-1 mature brown adipocytes versus control cells (“control”).

FIG. 24A depicts the generation of immortalized human brown and white fat progenitors. FIG. 24B provides a graphic depiction of UCP1 expression by FGF6 in human brown fat progenitors.

FIG. 25 depicts UCP1, PTGS2, LDHA, PDK1 and PKM2 gene expression in murine brown preadipocyte WT-1 cells following treatment with PGE2, PGI2 or FGF6 for 24 hours.

FIG. 26 provides a graphic description of the suppression of FGF6-induced UCP1 expression in WT-1 preadipocytes by AH-23848 (“AH”), a PGE2-EP4 receptor inhibitor. In the absence of AH, UCP1 expression is induced by FGF6 in WT-1 cells.

FIGS. 27A and 27B depict the effect of PGE2 treatment in WT-1 brown preadipocytes for 48 hours. FIG. 27A depicts a profile of cellular respiration of PGE2 treated brown preadipocytes (WT-1 cells) versus control cells (“buffer”) developed by utilizing well-characterized mitochondrial toxins including, oligomycin, an inhibitor of ATP synthase, which allows measurement of ATP turnover; an uncoupler, FGGP, was used to measure respiratory capacity; and a complex 1 inhibitor, rotenone, that prevents electron transfer activity and leaves only non-mitochondrial activity to be measured. The graph includes the buffer control; FGF6 exposed cells; PGI2; and PGE2. Also indicated are time points representing addition of pyruvate; 0.5 μM oligo; 1 μM FGGP; and 0.11 μM rotenone/2.2 μM ANT. FIG. 27B graphically describes glucose uptake in WT-1 cells exposed to either buffer control, 200 ng of FGF6, or 200 ng of PGE2 for 48 hours. Glucose levels in the “Ins0” columns were not exposed to insulin, while the cells in the “Ins100” columns were exposed to 100 nM of insulin.

FIG. 28A depicts the loss of FGFR1 and FGFR4 expression upon stable transfection of FGFR1 or FGFR4-specific siRNA in preadipocytes. The scramble control is a non-specific siRNA. FIG. 28B graphically depicts the loss of FGF6-mediated induction of UCP1 expression upon stable transfection of FGFR1-specific siRNA.

FIG. 29A depicts the suppression of FGF6-induced UCP1 expression in WT-1 preadipocytes following treatment with EX, a SIRT1 inhibitor. FIG. 29B depicts the suppression of FGF6-induced PTGS2 expression in WT-1 preadipocytes following treatment with EX.

FIG. 30 depicts the in vivo induction of UCP1 expression in subcutaneous white adipose tissue (SQ) and brown adipose tissue (BAT) upon injection of lenti-FGF6 virus into UCP1 reporter mice.

FIG. 31 graphically shows that FGF6 protein injection into C57BL6 mice fed either a Chow diet (FIG. 31A) or a high fat diet (FIG. 31B) lowers glucose levels relative to control mice injected with buffer.

FIG. 32 graphically depicts glucose levels in mice fed either the Chow diet (FIG. 32A) or a high fat diet (FIG. 32B) who were injected with FGF6 protein and insulin (insulin tolerance test). Panels on the right of the figure are the results of the left panels with normalization to initial blood glucose level (t=0 minutes).

FIG. 33 graphically depicts that injection of mice fed either the Chow diet (FIG. 33A) or a high fat diet (FIG. 33B), with FGF6 protein resulted in enhanced glucose tolerance, as seen in the lower levels of glucose in the FGF6 protein injected mice.

### DETAILED DESCRIPTION OF THE INVENTION

The present invention provides, in one embodiment, methods and compositions for the induction of UCP1 expression in cells such that the cell is converted to an energy consuming cell independent of adipocyte differentiation or lipid accumulation. The present invention also features, in one embodiment, methods and compositions for treating a disorder that would benefit from metabolic control, e.g., obesity or diabetes, comprising administering an FGF protein to a subject in need thereof.

In order that the present invention may be more readily understood, certain terms are first defined.

### I. DEFINITIONS

As used herein, the term “fibroblast growth factor” or “FGF” refers to a family of structurally related, heparin binding polypeptides, which are expressed in a wide variety of cells and tissues. Overall, the FGFs share between 17-72% amino acid sequence homology and a high level of structural similarity. A homology core of around 120 amino acids is highly conserved and has been identified in all members of the FGF family. The residues of the core domain interact with both the FGFR and heparin. Twelve antiparallel β strands have been identified in the core structure, labeled β1 through β12, linked one to another by loops of variable lengths, organized into a trefoil internal symmetry. Unless otherwise specified, the term “FGF” refers to both an FGF protein, or functional fragment, and a nucleic acid encoding an FGF protein, or functional fragment, e.g., “FGF6” indicates both the FGF6 protein and a nucleic acid encoding the FGF6 protein, as well as functional fragments that retain the ability to induce UCP1 expression. In one embodiment, FGF proteins bind to and activate an FGF receptor (FGFR). Characteristics of specific FGF proteins and subfamilies within the FGF family are described in more detail below in Section II.

As used herein, the term “FGF receptor agonist” refers to an agent that is capable of activating an FGF receptor. Examples of an FGF receptor agonist include, but are not limited to, an FGF protein (or functional fragment thereof), a nucleic acid encoding an FGF protein (or functional fragment thereof), an FGF mimetic or an anti-FGF receptor agonist antibody, or antigen binding fragment thereof. In one embodiment, the FGF receptor agonist is an agonist of FGFR1, such as an anti-FGFR1 agonist antibody.

As used herein, “UCP”, “UCP1” or “uncoupling protein 1”, is intended to refer to a 32 kDa inner mitochondrial transmembrane protein (or the gene which encodes the protein) expressed in brown adipocytes. UCP1 allows protons in the mitochondrial intermembrane space to re-enter the mitochondrial matrix without generating ATP, i.e., uncoupling.

As used herein, the term “UCP1 expression”, refers to detecting transcription of the gene encoding uncoupling protein 1 (UCP1), i.e., UCP1 mRNA or detecting translation of UCP1 mRNA, i.e., UCP1 protein. Thus, UCP1 expression, as used herein, refers to the presence of UCP1 in either protein or nucleic acid form, unless otherwise specified.

The term “cell”, as used herein, refers to an animal cell and not a plant cell.

As used herein, the term “differentiated cell” refers to a cell that is a mature cell, or a cell that has a defined morphology. An example of a differentiated cell includes, but is not limited to, a mature adipocyte.

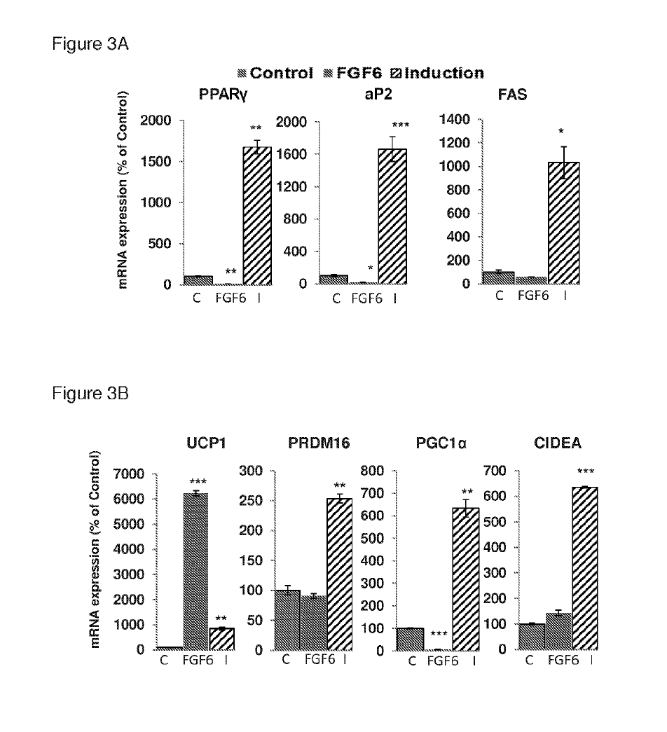

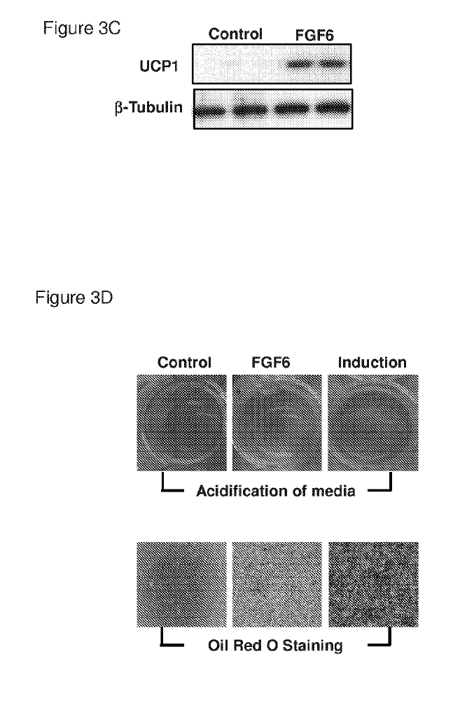

As used herein, the term “undifferentiated cell” refers to a cell that has not yet assumed a morphological or functional feature of a mature cell (a mature cell being the cell type at the end of a cell lineage). In one embodiment, an undifferentiated cell is a pluripotent cell that is capable of differentiating into cells of functionally distinct lineages. In one embodiment, the undifferentiated cell is an undifferentiated fibroblast cell. In another embodiment, the undifferentiated cell has the potential to express UCP1 upon exposure to an FGF. In yet another embodiment, an undifferentiated cell is a preadipocyte. In a further embodiment, the undifferentiated cell does not exhibit substantial lipid accumulation. In one embodiment, an undifferentiated cell is a cell committed to adipocyte lineage (general adipocyte lineage and determination is known in the art, e.g., general lineage is described in FIG. 3 of Tseng, Cypress, and Kahn (2010) Nat Rev Drugs and Dis. 9:465-482). As used herein, the term “cell committed to adipocyte lineage” refers to a cell which becomes an adipocyte when exposed to factors that induce adipogenic differentiation. In one embodiment, when the cell committed to adipocyte lineage is exposed to factors that induce, for example myogenic or osteogenic differentiation, it does not become a myocyte or an osteocyte, respectively.

As used herein, a “preadipocyte” refers to an adipocyte precursor cell that can proliferate and differentiate to form mature adipocytes. In one embodiment, a preadipocyte is a brown preadipocyte (e.g., WT-1 cell). In one embodiment, a preadipocyte is a white preadipocyte. In one embodiment, a preadipocyte can mature into a beige (also known as brite) adipocyte. The term “progenitor” is also used herein to describe a preadipocyte when used in the context of fat cells.

As used herein, “brown adipocytes”, “brown adipose tissue” or “BAT”, refers to a mature cell (or tissue thereof) characterized by multiple small lipid droplets and abundant mitochondria that oxidizes nutrients and generates heat. Central to the thermogenic activity of BAT is the expression of UCP1.

As used herein, the term “energy consuming cell” refers to a cell in which UCP1 expression is induced by an FGF, and has increased levels of mitochondrial respiration, such as basal respiration, ATP turnover, proton leak, and respiratory capacity. The levels of mitochondrial respiration of an energy consuming cell may be relative to a baseline respiration measure when UCP1 is not induced in the same cell type. In one embodiment, the level of UCP1 expressed in the cell is increased by at least about 3-fold, 4-fold, 5-fold, 6-fold, 10-fold, 15-fold, 20-fold, 25-fold, 30-fold, 35-fold, 40-fold, 45-fold, 50-fold, 75-fold, 100-fold, 200-fold, 300-fold, 400-fold, 500-fold, 1000-fold, 1500-fold, 2000-fold, 2500-fold, 3000-fold, 3500-fold, 4000-fold, 4500-fold, 5000-fold, 5500-fold, 6000-fold, 7000-fold, 8000-fold, 9000-fold or 10000-fold over baseline levels of the same type of cell in which UCP1 is not induced.

As used herein, the term “FGF-receptive cell” refers to a cell which can express UCP1 when contacted with an FGF receptor agonist, such as, but not limited to, FGF6. In one embodiment, an FGF-receptive cell is a cell which expresses an FGF receptor on its surface and expresses UCP1 when the FGF receptor (e.g., FGFR1) is contacted with an FGF receptor agonist (e.g., FGF6). The FGF-receptive cell can be, for example, an undifferentiated cell, e.g., a preadipocyte, or a differentiated cell, e.g., an adipocyte. In one embodiment, an FGF-receptive cell is an undifferentiated cell. In one embodiment, the FGF-receptive cell is a differentiated cell. In another embodiment, an FGF-receptive cell is a cell committed to adipocyte lineage. In one embodiment, a myogenic progenitor is not an FGF-receptive cell as it is unable to substantially express UCP1 when contacted with FGF6.

As used herein, the term “adipogenic marker” is intended to refer to proteins or RNA that are expressed during differentiation of progenitor cells, e.g., a preadipocyte, into an adipocyte.

As used herein, the term “lipid accumulation”, refers to the presence of lipid droplets within the cytoplasm of a cell, such as adipocytes. Lipid accumulation is most commonly found in adipocytes and represents the differentiated state of a fat cell. Substantial lipid accumulation is equivalent to lipid accumulation in an adipocyte cell, i.e., lipid accumulation in a differentiated fat cell.

In certain embodiments, the term “control”, as used herein, is intended to refer to a cell which is not contacted with an FGF receptor agonist. For example, a control may include a brown fat progenitor cell cultured using the same cell culture conditions, including the same culture media, but which is not contacted with an FGF. Alternatively, a control may refer to an FGF-receptive cell which is contacted with an induction media, but is not contacted with an FGF. The control may be used as a baseline in determining whether UCP1 expression is increased.

As used herein, the terms “induction conditions” and “differentiation conditions” refer to an environment which promotes cell differentiation. The term “induction media”, as used herein, refers to a solution having a compound or combination of compounds known to induce cell differentiation. Nonlimiting examples of compounds or compositions known to promote cell differentiation that may be used in induction media, or induction conditions, herein include dexamethasone and or 3-isobutyl-1-methylxanthine (IBMX). In one embodiment, preadipocytes are induced to differentiate by exposing the cells to bone morphogenetic protein 7 (BMP7). In a particular embodiment, preadipocytes are induced to differentiate by exposing the cells to BMP7, insulin and triiodothyronine (T3) in growth media (e.g., Dulbecco's Modified Eagles Medium (DMEM) and 10% fetal bovine serum (FBS)). In another particular embodiment, preadipocytes are induced to differentiate by exposing the cells to IBMX, dexamethasone, insulin and T3 in growth media (e.g., DMEM and 10% FBS).

As used herein “a disorder that would benefit from metabolic control” is intended to refer to diseases, disorders or conditions, lacking in metabolic regulation. A disorder that would benefit from metabolic control includes conditions where catabolism and/or anabolism are not effective in a subject (relative to known medical standards for a healthy population).

As used herein, the term “isolated” refers to a molecule, e.g., a protein or nucleic acid, which is separated from other molecules that are present in the natural source of the molecule. In one embodiment, an “isolated” molecule is substantially free of other cellular material, or culture media when produced by recombinant techniques, or, in the alternative, substantially free of chemical precursors or other chemicals when chemically synthesized. A molecule that is substantially free of cellular material includes preparations having less than about 30%, 20%, 19%, 18%, 17%, 16%, 15%, 14%, 13%, 12%, 11%, 10%, 9%, 8%, 7%, 6%, or about 5% of heterologous molecules and which retains the biological activity the molecule.

As used herein, the term “vector” refers to a nucleic acid molecule capable of transporting another nucleic acid to which it has been linked.

As used herein, the term “mimetic” when made in reference to a protein refers to a molecular structure which serves as a substitute for an FGF protein used in the present invention (see Morgan et al. (1989) Ann. Reports Med. Chem, 24:243-252 for a review of peptide mimetics). In one embodiment, a mimetic may be an organic compound that imitates the binding site of a specific FGF protein, and, therefore, the functionality of the FGF protein, e.g., inducing expression of UCP1 in an FGF-receptive cell.

The term “isostere”, as used herein, is intended to include a chemical structure that can be substituted for a second chemical structure because the steric conformation of the first structure fits a binding site specific for the second structure. The term specifically includes peptide backbone modifications (i.e., amide bond mimetics) well known to those skilled in the art. Such modifications include modifications of the amide nitrogen, the α-carbon, amide carbonyl, complete replacement of the amide bond, extensions, deletions or backbone crosslinks, Several peptide backbone modifications are known, including ψ[CH₂S], ψ[CH₂NH], ψ[CSNH₂], ψ[NHCO], ψ[COCH₂], and ψ[(E) or (Z) CH═CH]. In the nomenclature used above, Iv indicates the absence of an amide bond. The structure that replaces the amide group is specified within the brackets. Other examples of isosteres include peptides substituted with one or more benzodiazepine molecules (see e.g., James, C. L. et al. (1993) Science 260:1937-1942).

The term “antibody”, as used herein, is intended to refer to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH 1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from aminoterminus to carboxy-terminus in the following order: FR1, CDR1, FR1, CDR2, FR3, CDR3, FR4.

The term “antigen-binding portion” or “antigen-binding fragment” of an antibody (or simply “antibody portion”), as used herein, refers to a portion of a full-length antibody, generally the target binding or variable region. Examples of antibody fragments include Fab, Fab′, F(ab′)₂ and Fv fragments. The phrase “functional fragment” of an antibody is a compound having qualitative biological activity in common with a full-length antibody. For example, a functional fragment of an anti-FGF receptor antibody is one which can bind to an FGF receptor in such a manner so as to activate UCP1 expression in the cell. As used herein, “functional fragment” with respect to antibodies, refers to Fv, F(ab) and F(ab′)₂ fragments. An “Fv” fragment is the minimum antibody fragment which contains a complete target recognition and binding site. This region consists of a dimer of one heavy and one light chain variable domain in a tight, non-covalent association (VH-VL dimer). It is in this configuration that the three CDRs of each variable domain interact to define a target binding site on the surface of the VH-VL dimer. Collectively, the six CDRs confer target binding specificity to the antibody. However, even a single variable domain (or half of an Fv comprising only three CDRs specific for a target) has the ability to recognize and bind target, although at a lower affinity than the entire binding site.

The term “subject” or “patient,” as used herein interchangeably, refers to either a human or non-human animal. In one embodiment, the subject is a human.

The term “dose,” as used herein, refers to an amount of an FGF receptor agonist, (e.g., an FGF protein, or functional fragment thereof, a nucleic acid encoding an FGF protein, or functional fragment thereof, an FGF mimetic, an anti-FGF receptor agonist antibody, or antigen binding fragment thereof) or a cell in which UCP1 has been induced via contact with an FGF, which is administered to a subject.

The term “dosing”, as used herein, refers to the administration of a substance (e.g., an FGF protein, or fragment thereof, a nucleic acid encoding an FGF protein, an FGF mimetic, an anti-FGF receptor agonist antibody, or antigen binding fragment thereof, or a cell contacted with FGF) to achieve a therapeutic objective (e.g., the treatment of a disorder of glucose control, a disorder of weight control, a disorder of appetite control or obesity).

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present invention, therefore, includes methods of combination therapeutic treatment and combination pharmaceutical compositions.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of second or additional agents, wherein the second or additional agents, for example, may have been previously administered. A concomitant therapeutic treatment method may be executed step-wise by different actors. For example, one actor may administer to a subject a first agent and a second actor may to administer to the subject a second agent, and the administering steps may be executed at the same time, or nearly the same time, or at distant times, so long as the first agent (and additional agents) are after administration in the presence of the second agent (and additional agents). The actor and the subject may be the same entity (e.g., human).

The term “combination therapy”, as used herein, refers to the administration of two or more therapeutic substances, e.g., an FGF receptor agonist (e.g., an FGF protein, or fragment thereof, a nucleic acid encoding an FGF protein, an FGF mimetic, an anti-FGF receptor agonist antibody, or antigen binding fragment thereof) and another drug. The other drug(s) (e.g., a diabetic therapy, a HMG-CoA reductase inhibitor) may be administered concomitant with, prior to, or following the administration of an FGF receptor agonist, or a cell in which UCP1 expression has been induced via contact with an FGF. In contrast, use of the phrase “in the absence of” when referring to the combination of two or more therapeutic agents, e.g., an FGF receptor agonist and an additional growth factor, indicates that the two agents are not used in a combination therapy, as defined herein.

The term “kit” as used herein refers to a packaged product comprising components for administering a cell in which UCP1 expression has been induced via contact with an FGF or an FGF receptor agonist (e.g., an FGF protein, or fragment thereof, a nucleic acid encoding an FGF protein, an FGF mimetic, or an anti-FGF receptor agonist antibody, or antigen binding fragment thereof) of the invention for treatment of disorders that would benefit from metabolic control, e.g., diabetes or obesity. The kit preferably comprises a box or container that holds the components of the kit. The box or container is affixed with a label or a Food and Drug Administration approved protocol. The box or container holds components of the invention that are preferably contained within plastic, polyethylene, polypropylene, ethylene, or propylene vessels. The vessels can be capped-tubes or bottles. The kit can also include instructions for administering the cell or the FGF receptor agonist for use in the methods of the invention.

### II. METHODS AND COMPOSITIONS OF THE INVENTION

The present invention provides methods and compositions for the induction of Uncoupling Protein 1 (UCP1) expression in cells such that the cell is converted to an energy consuming cell independent of differentiation. As described herein, an FGF-receptive cell may be converted to an energy consuming cell regardless of whether or not it is a brown adipose tissue (BAT) cell, the cell type traditionally associated with energy expenditure. Thus, the present invention is based, at least in part, on the observation described herein that cells other than BAT cells can express UCP1 and be converted into energy and glucose consumers. In a further embodiment, the present invention is based on the discovery that mitochondrial activity, e.g., energy expenditure, can be increased in mature brown fat cells by exposure to an FGF, e.g., FGF6. The present invention further provides methods and compositions for the induction of UCP1 expression in cells such that the cell is converted to an energy consuming cell independent of substantial lipid accumulation.

In certain embodiments, the present invention takes advantage of the therapeutic potential of brown adipose tissue (BAT) or brown fat, as BAT has the capacity to dissipate energy and regulate triglyceride and glucose metabolism. The capacity of BAT to consume energy is due, in large part, to the expression of UCP1. The present invention is based, at least in part, on the discovery that FGFs can induce UCP1 expression in a cell without differentiation. The present invention is also based, at least in part, on the discovery that FGFs can induce UCP1 expression in a cell without substantial lipid accumulation. Thus, the methods of the invention include, but are not limited to, contacting an FGF-receptive cell, e.g., an undifferentiated cell or a differentiated cell, with an FGF receptor agonist (e.g., an FGF protein, or fragment thereof, a nucleic acid encoding an FGF protein, an FGF mimetic, or an anti-FGF receptor agonist antibody, or antigen binding fragment thereof, such as an anti-FGFR1 agonist antibody), or a cell in which UCP1 expression has been induced via contact with an FGF in an amount sufficient to induce UCP1 expression.

In one embodiment, the invention provides methods of expressing UCP1 in an FGF-receptive cell by contacting the FGF-receptive cell with an FGF receptor agonist, in an amount sufficient to induce UCP1 expression, wherein the FGF-receptive cell, e.g., a preadipocyte, does not differentiate following contact with the FGF receptor agonist.

In one embodiment, the invention provides methods of expressing UCP1 in an FGF-receptive cell by contacting the FGF-receptive cell with an FGF receptor agonist (e.g., an FGF protein, a nucleic acid encoding an FGF protein, an FGF mimetic, or an anti-FGFR1 agonist antibody, or antigen binding fragment thereof), in an amount sufficient to induce UCP1 expression. In certain embodiments, the FGF-receptive cell does not, however, differentiate following contact with the FGF. In some embodiments, the FGF-receptive cell is able to express UCP1 in the absence of agents associated with cell differentiation, e.g., growth factors. The FGF-receptive cell may be, for example, an undifferentiated cell. In another embodiment, the FGF-receptive cell may be, for example, a differentiated cell.

The invention further features methods of expressing UCP1 in a preadipocyte by contacting the preadipocyte with an FGF in an amount sufficient to induce UCP1 expression. Preferably, the preadipocyte does not differentiate into a brown adipocyte following contact with the FGF protein or nucleic acid encoding the FGF protein. While FGFs are known to promote brown fat cell differentiation, FGFs are not previously known to be able to express UCP1 expression in undifferentiated cells, including preadipocytes. The induction of UCP1 expression in undifferentiated cells results in an increase in mitochondrial respiration independent of (and in the absence of) differentiation.

In one embodiment, the invention features a method of increasing energy expenditure in a mature cell, e.g., a mature brown adipocyte, by exposing the cell to an FGF such that energy expenditure is increased. Such increase in energy consumption by the mature cell may be in the absence of increased UCP1 gene expression.

Contacting of the cell with the FGF receptor agonist, such as an FGF, e.g., FGF6, may be done directly or indirectly. Contacting the cell with the FGF receptor agonist may be performed either in vivo or in vitro. In certain embodiments, the cell is contacted with the FGF receptor agonist in vitro and subsequently transferred into a subject in an ex vivo method of administration. Contacting a cell with an FGF receptor agonist in vivo may be done, for example, by injecting the FGF receptor agonist into or near the tissue where the cell is located, or by injecting the FGF receptor agonist into another area, e.g., the bloodstream or the subcutaneous space, such that the FGF receptor agonist will subsequently reach the tissue where the cell to be contacted is located.

In certain embodiments of the invention, contacting a cell in vitro may be done by incubating the cell with an FGF receptor agonist. In one embodiment, the in vitro contact may occur by incubating an FGF-receptive cell with an FGF receptor agonist for a period of time, such as, for example, about 1 hour, about 2 hours, about 4 hours, about 8 hours, about 24 hours, about 48 hours, about 72 hours, about 96 hours, about 120 hours, about 144 hours, about 168 hours, or longer than 168 hours, or ranges thereof, in order to induce UCP1 expression.

In one embodiment of the invention, contacting a cell with an FGF receptor agonist includes introducing or delivering the FGF receptor agonist into the cell by facilitating or effecting uptake or absorption into the cell either in vivo or in vitro. For example, absorption or uptake of an FGF protein, a nucleic acid encoding an FGF protein, an FGF mimetic, or an anti-FGF receptor agonist antibody can occur through unaided diffusive or active cellular processes, or by auxiliary agents or devices. For example, for in vivo introduction, FGF protein a nucleic acid encoding an FGF protein, an FGF mimetic, or an anti-FGF receptor agonist antibody can be injected into a tissue site (e.g., brown or white adipose tissue) or administered systemically. In certain embodiments, the FGF receptor agonist is an anti-FGFR1 agonist antibody. In vitro introduction into a cell includes methods known in the art such as electroporation and lipofection. Further approaches are described in the Examples below.

In certain embodiments of the invention, the FGF-receptive cell that can express UCP1 and has an FGF receptor(s) on its surface is an undifferentiated cell. Non-limiting examples of an undifferentiated cell that may be used in the invention include a primary adipose precursor derived from brown and/or white fat, an adult stem cell, an embryonic stem cell, an induced pluripotent stem cell, a primary adipose progenitor found in stromal vascular fraction cells isolated from interscapular brown adipose tissue, a stromal-vascular fraction cell, a primary adipose progenitor (e.g., a primary adipose progenitor found in stromal vascular fraction cells isolated from subcutaneous white adipose tissue), an immortalized human brown fat precursor or progenitor cell, an immortalized human white fat precursor or progenitor cell, a murine brown preadipocyte cell (e.g., WT-1 cells), a white preadipocyte cell (e.g., a murine white preadipocyte cell such as 3T3-F442A cells), a brown preadipocyte, a white preadipocyte or a purified primary adipose precursor. A primary adipose precursor may be identified by FACS sorting as Sca-1+/CD45−/Mac1−/CD31.

Other examples of cells that are included in the methods and compositions of the invention are differentiated cells. A non-limiting example of a differentiated cell that may be used in the invention includes a mature adipocyte, including a white adipocyte, a brown adipocyte, and a beige adipocyte.

Another aspect of the invention is a method of generating immortalized human fat progenitors. In one embodiment, the fat progenitor is a human brown fat progenitor. In another embodiment, the fat progenitor is a human white fat progenitor. The method includes obtaining primary stromal-vascular fraction (SVF) cells from a human subject, and infecting the SVF cells with a virus that expresses human telomere reverse transcriptase (hTERT), such that immortalized human fat progenitors are generated. In one embodiment, the SVF cells are infected with the hTERT expressing virus at about 80% confluence. In one embodiment, the SVF cells are infected with the hTERT expressing virus until the SVF cells reach about 90% confluence. In a further embodiment, the SVF cells are infected with the virus in the presence of polybrene. Example 14 below describes an example of how to generate immortalized human fat progenitors in accordance with that aspect of the invention.

The methods of the invention include increasing UCP1 expression such that the FGF-receptive cell or tissue consumes energy. An increase in UCP1 expression can be detected using a number of methods described herein and known in the art. Detection of UCP1 mRNA or protein presence in a cell or tissue is reflective of UCP1 expression and can be quantified. Detecting and/or quantitating expression can include determining whether UCP1 expression is upregulated as compared to a control level, downregulated as compared to a control level, or substantially unchanged as compared to a control level. Therefore, the step of quantitating and/or detecting expression does not require that expression of UCP1 actually is upregulated or downregulated, but rather, can also include detecting no expression of UCP1 or detecting that the expression of UCP1 has not changed or is not different e.g., detecting no significant expression of UCP1 or no significant change in expression of UCP1 as compared to a control). In one embodiment, UCP1 expression in an FGF-receptive cell contacted with an FGF receptor agonist is compared to a control which is UCP1 expression in an FGF-receptive cell not contacted with an FGF receptor agonist. In another embodiment, UCP1 expression in an FGF-receptive cell contacted with an FGF receptor agonist is compared to UCP1 expression in a control which is an FGF-receptive cell contacted with induction media. In another embodiment, UCP1 expression in an FGF-receptive cell contacted with an FGF receptor agonist is compared to a control which is UCP1 expression in an FGF-receptive cell contacted with an adrenergic agonist.

In one embodiment, UCP1 expression occurs within a time period following exposure of the FGF-receptive cell or tissue to the FGF receptor agonist. For example, UCP1 expression may occur within about 4 hours, about 8 hours, about 24 hours, about 48 hours, about 72 hours, about 96 hours, about 120 hours, about 144 hours, or about 168 hours from contact of the cell with the FGF receptor agonist.

As discussed above, one of the surprising aspects of the present invention is the discovery that an undifferentiated cell contacted with an FGF receptor agonist shows increased UCP1 expression such that the cell is converted to an energy consuming cell and demonstrates high levels of mitochondrial metabolism in the absence of differentiation, e.g., to a brown adipocyte. The state of differentiation of the cell contacted with the FGF protein can be determined by measuring the level or amount of expression of markers, such as general adipogenic makers, brown adipocyte markers or inducible brown/beige/brite fat markers.

In one embodiment, the FGF-receptive cell which is contacted with the FGF receptor agonist does not exhibit substantial increases in expression of an adipogenic markers (markers indicating differentiation of adipocytes), including, but not limited to, Peroxisome Proliferator-Activated Receptor Gamma (PPARγ), Apatela 2 (aP2) and Apoptosis Antigen 1 (FAS, APO-1 or APT) For example, following contact of the undifferentiated cell with the FGF receptor agonist, the undifferentiated cell expresses levels or amounts of PPARγ, aP2 and FAS that are equivalent to a control cell (e.g., a cell not contacted with the FGF) or, alternatively, has lower levels of PPARγ, aP2 and FAS expression as compared to a control which is an undifferentiated cell that was contacted with the same FGF receptor agonist and an agent (or media) which can induce differentiation. For example, following contact of the undifferentiated cell with the FGF receptor agonist, the undifferentiated cell may express levels or amounts of PPARγ that are at least about 100-fold, 200-fold, 300-fold, 400-fold, 500-fold, 800-fold, 900-fold, 1000-fold, 1500-fold, 2000-fold or 2500-fold lower than the level or amount expressed by the same type of cell contacted with induction media and the FGF receptor agonist. Ranges within one or more of the preceding values, e.g., about 100-fold to about 500-fold, about 400-fold to about 800-fold, about 600-fold to about 1000-fold, about 800-fold to about 1500-fold, about 1000-fold to about 2000-fold or about 100-fold to about 2500-fold are contemplated by the invention. In another example, following contact of the undifferentiated cell with the FGF receptor agonist, the undifferentiated cell may express levels or amounts of aP2 that are at least about 100-fold, 200-fold, 300-fold, 400-fold, 500-fold, 800-fold, 900-fold, 1000-fold, 1500-fold or 2000-fold lower than the level or amount expressed by the same type of cell contacted with induction media and the FGF receptor agonist. Ranges within one or more of the preceding values, e.g., about 100-fold to about 500-fold, about 400-fold to about 800-fold, about 600-fold to about 1000-fold, about 800-fold to about 1500-fold, about 1000-fold to about 2000-fold or about 100-fold to about 2000-fold are contemplated by the invention. In another example, following contact of the undifferentiated cell with the FGF receptor agonist, the undifferentiated cell may express levels or amounts of FAS that are at least about 2-fold, 3-fold, 4-fold, 5-fold, 6-fold, 7-fold, 8-fold, 9-fold, 10-fold, 15-fold of 20-fold lower than the level or amounts expressed by the same type of cell contacted with induction media and the FGF receptor agonist. Ranges within one or more of the preceding values, e.g., about 2-fold to about 5-fold, about 4-fold to about 8-fold, about 6-fold to about 10-fold, about 8-fold to about 15-fold, about 15-fold to about 20-fold, or about 2-fold to about 20-fold are contemplated by the invention.

In one embodiment, the FGF-receptive cell that is contacted with the FGF receptor agonist does not exhibit a substantial increase in expression of brown fat or brown adipocyte marker(s) (markers whose presence indicates differentiation of brown adipocytes) indicative of differentiation. Examples of such markers include, but are not limited to PR Domain Containing 16 (PRDM16), PPAR-gamma Coactivator 1 (PGC1), and Cell Death Inducing DFFA-Like Effector A (CIDEA). The expression level of a brown adipocyte marker(s) in the FGF receptor agonist-contacted cell can be compared to the expression level in a control that has not been contacted with an FGF receptor agonist or induction media, wherein equivalent levels of expression would be expected in the absence of differentiation. Alternatively, the expression level of a brown adipocyte marker(s) in the FGF receptor agonist-contacted cell can be compared to the expression level in a control that has been contacted with the FGF receptor agonist and exposed to induction media, wherein lower levels of expression in the FGF receptor agonist-exposed cell, versus the FGF receptor agonist-exposed+induction media exposed cell, would indicate a lack of differentiation. For example, following contact of the undifferentiated cell with an FGF receptor agonist, the undifferentiated cell may express levels or amounts of PRDM16 that are at least about 2-fold, 3-fold, 4-fold, 5-fold, 6-fold, 7-fold, 8-fold, 9-fold or 10-fold lower than the level or amount expressed by the same type of cell contacted with the FGF receptor agonist and induction media. Ranges within one or more of the preceding values, e.g., about 2-fold to about 5-fold, about 4-fold to about 8-fold, about 6-fold to about 10-fold or about 2-fold to about 10-fold are contemplated by the invention. In another example, following contact with an FGF receptor agonist, an undifferentiated cell may express levels or PGC1 that are at least about 100-fold, 200-fold, 300-fold, 400-fold, 500-fold, 600-fold, 700-fold, 800-fold, 900-fold or 1000-fold lower than the level or amount expressed by the same type of cell contacted with the FGF receptor agonist and induction media (thus resulting in a control cell which is a differentiated cell). Ranges within one or more of the preceding values e.g., about 100-fold to about 500-fold, about 400-fold to about 800-fold, about 600-fold to about 1000-fold or about 100-fold to about 1000-fold are contemplated by the invention. In another example, following contact with an FGF receptor agonist, an undifferentiated cell may express levels or amounts of CIDEA that are at least about 2-fold, 3-fold, 4-fold, 5-fold, 6-fold, 7-fold, 8-fold, 9-fold or 10-fold lower than the level or amount expressed by the same type of cell contacted with the FGF receptor agonist and induction media (thus resulting in a control cell which is a differentiated cell). Ranges within one or more of the preceding values, e.g., about 2-fold to about 5-fold, about 4-fold to about 8-fold, about 6-fold to about fold or about 2-fold to about 10-fold are contemplated by the invention.

In another embodiment, inducible brown/beige/brite fat markers may be used to determine differentiation (or lack thereof) in the cells of the invention. Examples of brown/beige/brite fat markers that may be used include, for example, Tbx1 (i.e., T box transcription factor 1), Tmem26 (i.e., Transmembrane Protein 26) and CD137 (i.e., tumor necrosis factor receptor superfamily member 9).

In one embodiment of the invention, contacting an FGF-receptive cell with an FGF receptor agonist induces expression of the PTGS2 gene and or the Cox2 protein. As used herein, the terms “PTGS2”, “COX2”, “COX-2” or “Cox2” are used interchangeably to refer to prostaglandin-endoperoxide synthase 2, also known as cyclooxygenase-2. The Cox2 enzyme is encoded by the PTGS2 gene. Cox2 is involved in the conversion of arachidonic acid to prostaglandin H2, an important precursor of prostacyclin and thromboxane A2, among others. COX2 expression is regulated by various stimuli. The COX2-PG pathway is transiently induced during early stage of adipogenesis (Fujimori K., PPAR Res 2012:527607, 2012), and was found to play a critical role in recruiting brown fat cells within white adipose tissue (Madsen L., et al., PLOS One 5:e11391, 2010; Vegiopoulos A. et al., Science 328:1158-1161, 2010). In one embodiment, the level or amount of PTGS2 expression may be increased by at least about 2-fold, 3-fold, 4-fold, 5-fold, 6-fold, 7-fold, 8-fold, 9-fold, 10-fold. 20-fold, 30-fold, 40-fold, 50-fold, 100-fold, 150-fold, 200-fold, 250-fold, 300-fold, 350-fold or 400-fold in an FGF-receptive cell following contact with an FGF receptor agonist relative to the level or amount of expression in the same type of cell not contacted with the FGF receptor agonist. Ranges within one or more of the preceding values e.g., about 2-fold to about 4-fold, about 3-fold to about 6-fold, about 5-fold to about 10-fold, about 8-fold to about 30-fold, about 20-fold to about 50-fold, about 40-fold to about 100-fold, about 50-fold to about 200-fold, about 200-fold to about 400-fold or about 2-fold to about 400-fold are contemplated by the invention.

In another embodiment of the invention, NRIP1 mRNA expression is decreased following contact of the FGF-receptive cell with an FGF. As used herein, the terms “receptor interacting protein 140”, “RIP-140”, “nuclear receptor interacting protein 1” or “NRIP1” is intended to refer to a nuclear co-regulator that controls a variety of physiological functions. In one embodiment, the gene plays a key role in the regulation of energy metabolism by repressing a number of nuclear receptors (Nautiyal J. et al., Trends Endocrinol Metab 24:451-459, 2013). For example, RIP140 knockout mice are lean with increased energy expenditure and are resistant to high-fat diet-induced obesity (Leonardsson G. et al., Proc Nati Acad Sci USA 101:8437-8442, 2004). The white adipose tissue of RIP140 knockout mice displays genes characteristic of brown adipose tissue, including UCP1 and CIDEA. At the molecular level, RIP140 directs histone and DNA methylation to silence UCP1 expression and suppress mitochondrial biogenesis in white adipocytes (Kiskinis E. et al., EMBO J 26:4831-4840, 2007; Powelka A. M. et al., J Clin Invest 116:125-136, 2006). RIP140 also interacts with liver X receptor α (LXRα) to suppress UCP1 gene expression and the brown fat phenotype (Wang H. et al., Mol Cell Biol 28:2187-2200, 2008). In one embodiment, the level or amount of NRIP1 expression is decreased by at least about 30%, 40%, 50%, 60%, 70%, 80%, 90%, or 100% in an FGF-receptive cell following contact with the FGF receptor agonist relative to the level or amount of expression in the same type of cell not contacted with the FGF receptor agonist. Ranges within one or more of the preceding values e.g., about 30% to about 50%, about 40% to about 70%, about 60% to about 100% or about 30% to about 100% are contemplated by the invention.

By exposing a cell, e.g., an undifferentiated cell, to an FGF receptor agonist, UCP1 expression can be induced in the absence of cell differentiation. Thus, while UCP1 is expressed in the undifferentiated cell, the undifferentiated cell does not exhibit certain characteristics found in differentiated cells. For example, if a preadipocyte is contacted with an FGF receptor agonist such that UCP1 expression is induced but differentiation does not occur, the preadipocyte will not accumulate substantial amounts of lipid like that found in mature adipocytes (or a differentiated fat cell). Lipid in a differentiated fat cell be may in the form of a single droplet (e.g., white adipocytes) or multiple, small droplets (e.g., multilocular droplets found in brown adipocytes). Lipid accumulation may be visualized using microscopy techniques well known in the art such as, for example, light microscopy (e.g., reverse phase, bright field) or electron microscopy. In some embodiments, the lipid accumulation may be further visualized using biological stains in combination with microscopy. Exemplary stains for detecting lipid accumulation include, but are not limited to, oil-red-O, Sudan III, Sudan IV, osmium tetroxide, and Sudan Black B.

In one embodiment of the invention, contact of the FGF-receptive cell with an FGF receptor agonist (e.g., an FGF protein, a nucleic acid encoding an FGF protein, an FGF mimetic, or an anti-FGF receptor agonist antibody, or antigen binding fragment thereof) converts the FOP-receptive cell into an energy consuming cell. Conversion to an energy consuming cell can be determined by detecting expression of UCP1 or quantifying expression of UCP1. For example, following contact of the FGF-receptive cell with the FGF receptor agonist, the FOE-receptive cell is converted to an energy consuming cell and expresses levels or amounts of UCP1 that are at least about 3-fold, 4-fold, 5-fold, 6-fold, 10-fold, 15-fold, 20-fold, 25-fold, 30-fold, 35-fold, 40-fold, 45-fold, 50-fold, 75-fold, 100-fold, 200-fold, 300-fold, 400-fold, 500-fold, 1000-fold, 1500-fold, 2000-fold, 2500-fold, 3000-fold, 3500-fold, 4000-fold, 4500-fold, 5000-fold, 5500-fold, 6000-fold, 7000-fold, 8000-fold, 9000-fold or 10000-fold higher than the level or amount expressed in cells contacted with induction media or than the level or amount expressed in control cells (e.g., FGF-receptive cells that have not been contacted with an FGF receptor agonist). Ranges within one or more of the preceding values, e.g., about 3-fold to about 10-fold, about 5-fold to about 50-fold, about 25-fold to about 200-fold, about 100-fold to about 1000-fold, about 500-fold to about 5000-fold, about 2500-fold to about 10000-fold or about 3-fold to about 10000-fold, are contemplated by the invention.

Conversion to an energy consuming cell can also be determined by measuring mitochondrial metabolism. For example, following contact of the FGF-receptive cell with the FGF receptor agonist (e.g., an FGF protein, a nucleic acid encoding an FGF protein, an FGF mimetic, or an anti-FGF receptor agonist antibody, or antigen binding fragment thereof), the EGF-receptive cell may demonstrate increased mitochondrial metabolism. To assess mitochondrial metabolism, mitochondrial activity can be measured using, for example, a Seahorse Bioanalyzer. For example, cells are provided with abundant nutrients (e.g., 10 mM glucose, 0.5 mM carnitine, and 1 mM palmitate-BSA) and a profile of cellular respiration is developed by utilizing well-characterized mitochondrial toxins. Basal respiration is measured, followed by injection of oligomycin, an inhibitor of ATP synthase, which allows measurement of ATP turnover. The uncoupler FCCP is injected to measure respiratory capacity, followed by the complex 1 inhibitor rotenone, which prevents electron transfer activity and leaves only non-mitochondrial activity to be measured. This allows the bioenergetic profile (i.e., mitochondrial metabolism), comprising basal respiration, ATP turnover, proton leak and respiratory capacity, of energy consuming cells to be measured. In one embodiment, the FGF-receptive cell demonstrates levels or amounts of mitochondrial metabolism that are about 1.5-fold, 2-fold, 2.5-fold, 3-fold, 3.5-fold, 4-fold, 5-fold, 6-fold, 7-fold, 8-fold, 9-fold, 10-fold, 12-fold, 15-fold, 18-fold, 20-fold, 22-fold or 25-fold higher than in a control cell (i.e., a cell not contacted with an FGF receptor agonist). Ranges within one or more of the preceding values e.g., about 1.5-fold to about 3-fold, about 2-fold to about 6-fold, about 3-fold to about 10-fold, about 5-fold to about 15-fold, about 12-fold to about 20-fold, about 15-fold to about 25-fold or about 1.5-fold to about 25-fold are contemplated by the invention.

In another embodiment, the FOE-receptive cell demonstrates levels or amounts of mitochondrial metabolism resulting from an increase in any one of basal respiration, ATP turnover, proton leak and/or respiratory capacity. For example, any one of basal respiration, ATP turnover, proton leak and/or respiratory capacity is increased by at least about 1.5-fold, 2-fold, 2.5-fold, 3-fold, 3.5-fold, 4-fold, 5-fold, 6-fold, 7-fold, 8-fold, 9-fold, 10-fold, 12-fold, 15-fold, 18-fold, 20-fold, 22-fold or 25-fold as compared to a control cell. Ranges within one or more of the preceding values e.g., about 1.5-fold to about 3-fold, about 2-fold to about 6-fold, about 3-fold to about 10-fold, about 5-fold to about 15-fold, about 12-fold to about 20-fold, about 15-fold to about 25-fold or about 1.5-fold to about 25-fold are contemplated by the invention.

The level of an mRNA encoding a marker described herein can be measured using methods known to those skilled in the art, e.g. Northern analysis. Gene expression of the marker can be detected at the RNA level. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilizing ribonucleic acid hybridization include nuclear run-on assays, RT-PCR, RNase protection assays (Melton et al., Nuc. Acids Res. 12:7035), Northern blotting and In Situ hybridization. Gene expression can also be detected by microarray analysis as described below.

For Northern blotting, RNA samples are first separated by size via electrophoresis in an agarose gel under denaturing conditions. The RNA is then transferred to a membrane, crosslinked and hybridized with a labeled probe. Nonisotopic or high specific activity radiolabeled probes can be used including random-primed, nick-translated, or PCR-generated DNA probes, in vitro transcribed RNA probes, and oligonucleotides. Additionally, sequences with only partial homology (e.g., cDNA from a different species or genomic DNA fragments that might contain an exon) may be used as probes.

In situ hybridization (ISH) is a powerful and versatile tool for the localization of specific mRNAs in cells or tissues. Hybridization of the probe takes place within the cell or tissue. Since cellular structure is maintained throughout the procedure, ISH provides information about the location of mRNA within the tissue sample. The procedure begins by fixing samples in neutral-buffered formalin, and embedding the tissue in paraffin. The samples are then sliced into thin sections and mounted onto microscope slides. (Alternatively, tissue can be sectioned frozen and post-fixed in paraformaldehyde.) After a series of washes to dewax and rehydrate the sections, a Proteinase K digestion is performed to increase probe accessibility, and a labeled probe is then hybridized to the sample sections. Radiolabeled probes are visualized with liquid film dried onto the slides, while nonisotopically labeled probes are conveniently detected with colorimetric or fluorescent reagents. This latter method of detection is the basis for Fluorescent In Situ Hybridisation (FISH).

Methods for detection which can be employed include radioactive labels, enzyme labels, chemiluminescent labels, fluorescent labels and other suitable labels.

Typically, real time (RT-PCR) (also called QPCR) is used to amplify RNA targets. In this process, the reverse transcriptase enzyme is used to convert RNA to complementary DNA (cDNA) which can then be amplified to facilitate detection. Relative quantitative RT-PCR involves amplifying an internal control simultaneously with the gene of interest. The internal control is used to normalize the samples. Once normalized, direct comparisons of relative abundance of a specific mRNA can be made across the samples. Commonly used internal controls include, for example, GAPDH, HPRT, actin and cyclophilin.

The methods of the invention may be performed using protein-based assays to determine the level of the given marker. Examples of protein-based assays include immunohistochemical and/or Western analysis, quantitative blood based assays, e.g., serum ELISA, and quantitative urine based assays, e.g., urine ELISA. In one embodiment, an immunoassay is performed to provide a quantitiative assessment of the given marker.

Proteins from samples can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

The amount of marker may be determined by detecting or quantifying the corresponding expressed polypeptide. The polypeptide can be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, and Western blotting.

The methods of the invention provide, in certain embodiments, a therapeutic means to treat metabolic disorders that would benefit from increased energy consumption, e.g., diabetes or obesity, attained through induction of UCP1. Examples of disorders that would benefit from metabolic control include, but are not limited to a disorder that would benefit from glucose control, a disorder that would benefit from weight control, a disorder that would benefit from cholesterol control, and a fatty acid metabolism disorder.

In one embodiment, the invention provides a method of treating a disorder that would benefit from glucose control comprising administering an FGF receptor agonist (or a cell contacted with an FGF receptor agonist such that UCP1 expression is induced) to a subject in need thereof. Examples of a disorder that would benefit from glucose control include, but are not limited to, insulin resistance, diabetes, hyperglycemia, and metabolic syndrome.

Diabetes is a disease which is marked by elevated levels of sugar (glucose) in the blood. Diabetes can be caused by too little insulin (a chemical produced by the pancreas to regulate blood sugar), resistance to insulin, or both. The methods and compositions of the invention may also be used to treat disorders associated with diabetes including, for example, hyperglycemia, hyperinsulinaemia, hyperlipidaemia, insulin resistance, impaired glucose metabolism, obesity, diabetic retinopathy, macular degeneration, cataracts, diabetic nephropathy, glomerulosclerosis, diabetic neuropathy, erectile dysfunction, premenstrual syndrome, vascular restenosis, ulcerative colitis, coronary heart disease, hypertension, angina pectoris, myocardial infarction, stroke, skin and connective tissue disorders, foot ulcerations, metabolic acidosis, arthritis, and osteoporosis.

Diabetes includes the two most common types of the disorder, namely type I diabetes and type II diabetes, which both result from the body's inability to regulate insulin. Insulin is a hormone released by the pancreas in response to increased levels of blood sugar (glucose) in the blood.

The term “type 1 diabetes,” as used herein, refers to a chronic disease that occurs when the pancreas produces too little insulin to regulate blood sugar levels appropriately. Type 1 diabetes is also referred to as insulin-dependent diabetes mellitus, IDMM, juvenile onset diabetes, and diabetes—type I. Type 1 diabetes represents is the result of a progressive autoimmune destruction of the pancreatic β-cells with subsequent insulin deficiency.

The term “type 2 diabetes,” refers to a chronic disease that occurs when the pancreas does not make enough insulin to keep blood glucose levels normal, often because the body does not respond well to the insulin. Type 2 diabetes is also referred to as noninsulin-dependent diabetes mellitus, NDDM, and diabetes—type II.

The methods and compositions of the invention may be used to treat both type I and type II diabetes, by providing a means to control glucose levels in the subject in need thereof.

Diabetes can be diagnosed by the administration of a glucose tolerance test. Clinically, diabetes is often divided into several basic categories. Primary examples of these categories include, autoimmune diabetes mellitus, non-insulin-dependent diabetes mellitus (type 1 NDDM), insulin-dependant diabetes mellitus (type 2 IDDM), non-autoimmune diabetes mellitus, non-insulin-dependant diabetes mellitus (type 2 NIDDM), and maturity-onset diabetes of the young (MODY). A further category, often referred to as secondary, refers to diabetes brought about by some identifiable condition which causes or allows a diabetic syndrome to develop. Examples of secondary categories include, diabetes caused by pancreatic disease, hormonal abnormalities, drug- or chemical-induced diabetes, diabetes caused by insulin receptor abnormalities, diabetes associated with genetic syndromes, and diabetes of other causes. (see e.g., Harrison's (1996) 14^{th} ed., New York, McGraw-Hill).

In another embodiment, the FGF receptor agonist (or a cell contacted with an FGF receptor agonist such that UCP1 expression is induced) is administered in combination with a diabetic therapy and/or a HMG-CoA reductase inhibitor. Exemplary diabetic therapies are known in the art and include, for example, insulin sensitizers, such as biguanides (e.g., metformin) and thiazolidinediones (e.g., rosiglitazone, pioglitazone, troglitazone); secretagogues, such as the sulfonylureas (e.g., glyburide, glipizide, glimepiride, tolbutamide, acetohexamide, tolazamide, chlorpropamide, gliclazide, glycopyamide, gliquidone), the nonsulfonylurea secretagogues, e.g., meglitinide derivatives (e.g., repaglinide, nateglinide); the dipeptidyl peptidase IV inhibitors (e.g., sitagliptin, saxagliptin, linagliptin, vildagliptin, allogliptin, septagliptin); alpha-glucosidase inhibitors (e.g., acarbose, miglitol, voglibose); amylinomimetics (e.g., pramlintide acetate); incretin mimetics (e.g., exenatide, liraglutide, taspoglutide); insulin and its analogues (e.g., rapid acting, slow acting, and intermediate acting); bile acid sequestrants (e.g., colesevelam); and dopamine agonists (e.g., bromocriptine), alone or in combinations. Exemplary HMG-CoA reductase inhibitors include atorvastatin (Pfizer's Lipitor®/Tahor/Sortis/Torvast/Cardyl), pravastatin (Bristol-Myers Squibb's Pravachol, Sankyo's Mevalotin/Sanaprav), simvastatin (Merck's Zocor®/Sinvacor, Boehringer Ingelheim's Denan, Banyu's Lipovas), lovastatin (Merck's Mevacor/Mevinacor, Bexal's Lovastatina, Cepa; Schwarz Pharma's Liposcler), fluvastatin (Novartis' Lescol®/Locol/Lochol, Fujisawa's Cranoc, Solvay's Digaril), cerivastatin (Bayer's Lipobay/GlaxoSmithKline's Baycol), rosuvastatin (AstraZeneca's Crestor®), and pitivastatin (itavastatin/risivastatin) (Nissan Chemical, Kowa Kogyo, Sankyo, and Novartis).

In one embodiment, the invention provides a method of treating a disorder that would benefit from weight control comprising administering an FGF receptor agonist (or a cell contacted with an FGF receptor agonist such that UCP1 expression is induced) to a subject in need thereof. Examples of a disorder that would benefit from weight control include, but are not limited to, liver disease, dyslipidemia, a glycemic control disorder, cardiovascular disease and obesity. Obesity refers to a condition in which the subject has an excess of body fat relative to lean body mass. In one embodiment, obesity refers to a condition in which an individual weighs at least about 20% or more over the maximum desirable for their height. When an adult is more than 100 pounds overweight, he or she is considered to be “morbidly obese.” In another embodiment, obesity is defined as a BMI (body mass index) over 30 kg/m2. Obesity increases a person's risk of illness and death due to diabetes, stroke, coronary artery disease, hypertension, high cholesterol, and kidney and gallbladder disorders. Obesity may also increase the risk for some types of cancer, and may be a risk factor for the development of osteoarthritis and sleep apnea. Obesity can be treated with the methods and compositions of the invention alone or in combination with other metabolic disorders, including diabetes. In another embodiment, a disorder that would benefit from metabolic control may be a disorder associated with obesity, for example, high blood pressure, diabetes, hyperglycemia, heart disease, high cholesterol, cancer, infertility, back pain, skin infections, gastric ulcers, gallstones, sleep apnea and osteoarthritis.

In one embodiment, the invention provides a method of treating a disorder that would benefit from cholesterol control comprising administering an FGF receptor agonist (or a cell contacted with an FGF receptor agonist such that UCP1 expression is induced) to a subject in need thereof. A disorder that would benefit from cholesterol control may be, for example, heart disease.

In one embodiment, the invention provides a method of treating a fatty acid metabolism disorder comprising administering an FGF receptor agonist (or a cell contacted with an FGF receptor agonist such that UCP1 expression is induced) to a subject in need thereof. Fatty acid metabolism disorder is characterized by difficulty breaking down (metabolizing) fatty acids. Examples of fatty acid metabolism disorder include but are not limited to, medium chain acyl CoA dehydrogenase deficiency (MCADD), long chain acyl CoA dehydrogenase deficiency (LCHADD), and very long chain acyl CoA dehydrogenase deficiency (VLCHADD).

Another exemplary disorder that would benefit from metabolic control is metabolic syndrome. Accordingly, in one embodiment, the invention provides a method of treating or preventing metabolic syndrome in a subject, comprising administering an FGF receptor agonist (or a cell contacted with an FGF receptor agonist such that UCP1 expression is induced) to a subject in need thereof. Metabolic syndrome is a cluster of conditions that occur together in various combinations. These conditions include elevated blood pressure, high blood sugar level, excess body fat around the waist, and abnormal cholesterol levels. A combination of the foregoing conditions can increase the risk that a subject will develop heart disease, stroke, and diabetes. Metabolic syndrome is linked to insulin resistance, and subjects having metabolic syndrome frequently display insulin resistance as well. A subject can be diagnosed as having metabolic syndrome if the subject displays three or more traits selected from a large waist circumference (e.g., at least about 35 inches for women and at least about 40 inches for men); a high triglyceride level (e.g., a triglyceride level of at least about 150 mg/dL, e.g., at least about 1.7 mmol/L); reduced levels of HDL cholesterol (e.g., a HDL level of less than about 40 mg/dL (e.g., less than about 1.04 mmol/L) in men, or a HDL level of less than about 50 mg/dL (e.g., less than about 1.3 mmol/L) in women); increased blood pressure (e.g., blood pressure of at least about 130/85 mmHg); and elevated fasting blood sugar (e.g. a fasting blood sugar level of at least about 100 mg/dL (e.g., at least about 5.6 mmol/L). In some embodiments, traits associated with metabolic syndrome can also include receiving treatment for high triglyceride level, receiving treatment for low HDL level, receiving treatment for high blood pressure, and/or receiving treatment for high blood sugar. A subject at risk of developing metabolic syndrome can be identified by determining if the subject displays at least one of the foregoing traits, and/or by determining if the subject has insulin resistance. In one embodiment, a subject is at risk of developing metabolic syndrome can be identified by determining if the subject displays at least two of the foregoing traits, and/or by determining if the subject has insulin resistance.

In certain embodiments, the methods described herein are beneficial for increasing energy expenditure in preadipocytes and/or mature adipocytes in order to achieve weight loss in a subject in need thereof (e.g., an obese subject), where the methods of the invention are used as a single therapy or in combination with other weight loss therapies, such as bariatric surgery. Thus, in one embodiment, the invention provides a method of achieving weight loss in a subject in need thereof, comprising administering an FGF receptor agonist, such as FGF6, to a subject, e.g., locally administering FGF6, prior to, during, or following bariatric surgery in the subject.

In one embodiment, the invention includes a method of treating a disorder that would benefit from metabolic control in a subject, comprising selecting a subject having or at risk for a disorder that would benefit from metabolic control, and administering an FGF receptor agonist (or a cell contacted with an FGF receptor agonist such that UCP1 expression is induced) to the subject.

In one aspect, a selection step is performed wherein a subject having a disorder recited herein is selected prior to the administration of the FGF receptor agonist, e.g., FGF6. For example, in one embodiment, a subject having metabolic syndrome is selected. In another embodiment, a subject in need of weight loss is selected for treatment.

In one embodiment, the invention includes a method of treating a disorder that would benefit from metabolic control in a subject, comprising administering an FGF receptor agonist (or a cell contacted with an FGF receptor agonist such that UCP1 expression is induced) to the subject, such that the disorder is treated, wherein the FGF receptor agonist is administered to the subject in the absence of an additional agent selected from the group consisting of an additional growth factor, dexamethasone, and indomethacin. Thus, in certain embodiments, the methods of the invention are performed without co-administering (at the same time or immediately before or after) an additional agent known to induce differentiation of adipocytes, such as growth factors.

In another aspect, the present invention provides methods treating a subject having diabetes or obesity. The method comprises administering a composition comprising an FGF6 protein or a nucleic acid encoding an FGF6 protein to the subject, such that the diabetes or obesity in the subject is treated, wherein the FGF6 protein or the nucleic acid encoding the FGF6 protein is administered to the subject in the absence of an additional agent selected from the group consisting of an additional growth factor, dexamethasone, and indomethacin.

The FGF receptor agonist used in the methods of the invention to treat a subject having a disorder that may benefit from metabolic control may be, for example, an FGF protein (or nucleic acid encoding an FGF protein) such as FGF1, FGF2, FGF4, FGF6, FGF5, FGF9, FGF16, FGF17, FGF18, and FGF20. In a specific embodiment, the FGF protein is FGF6. In another specific embodiment, the FGF protein is not FGF21. In other embodiments, the FGF receptor agonist used in the methods of the invention may be, for example, an anti-FGF receptor antibody, or an antigen-binding fragment thereof, that binds to and activates an FGF receptor. In an exemplary embodiment, the FGF receptor agonist used in the methods of the invention is an antibody, or antigen-binding fragment thereof, that binds to and activates FGF receptor 1 (FGFR1).

In some embodiments of the invention, the FGF receptor agonist (or a cell contacted with an FGF receptor agonist such that UCP1 expression is induced) is administered in combination with another agent. In one embodiment, a combination of FGF receptor agonists can be used in the methods of the present invention. For example, two or more FGF proteins (or a nucleic acid encoding an FGF protein) can be used in combination, specifically, FGF6 and FGF9, FGF6 and FGF2 or FGF9 and FGF2. In another embodiment the combination includes two or more FGF proteins selected from the group consisting of FGF1, FGF2, FGF4, FGF6, FGF8, FGF9, FGF16, FGF17, FGF18, and FGF20.

Typical modes of administration of the FGF receptor agonist (or a cell contacted with an FGF receptor agonist such that UCP1 expression is induced) include parenteral (e.g., intravenous, subcutaneous, intraperitoneal, intramuscular) injection or oral administration. In one embodiment, the FGF receptor agonist is administered by injection. In another embodiment, the injection is subcutaneous. In a particular embodiment, the injection is into adipose tissue.

In one embodiment of the invention, an FGF receptor agonist, such as, but not limited to FGF6 protein, is administered locally to white adipose tissue. Such administration may be, for example, subcutaneous. Local administration provides for increases in energy consumption in particular locations within a subject's body that may benefit from such energy use. Thus, the invention provides a means of reducing localized fat deposits in areas having, brown, white, and/or beige fat. Such areas of a subject that may benefit from local delivery of an FGF receptor agonist include thighs, hips, buttocks, abdomen, waist, upper arm, back, inner knee, chest area, cheeks, chin and neck, and calves and ankles. In one embodiment, locally delivery of an FGF receptor agonist in order to increase energy consumption of the fatty tissue is performed in combination with liposuction.

In another embodiment, the FGF receptor agonist (or cell contacted with an FGF receptor agonist such that UCP1 expression is induced) is administered at a dose of about 0.5 mg/kg to about 300 mg/kg. In one embodiment, the FGF receptor agonist (or a contacted with an FGF receptor agonist such that UCP1 expression is induced) is administered at a dose of about 1 mg/kg, 2 mg/kg, 3 mg/kg, 4 mg/kg, 5 mg/kg, 10 mg/kg, 20 mg/kg, 30 mg/kg, 40 mg/kg, 50 mg/kg, 100 mg/kg, 150 mg/kg, 200 mg/kg, 250 mg/kg, 300 mg/kg, 350 mg/kg, 400 mg/kg, 450 mg/kg or 500 mg/kg. Ranges within one or more of the preceding values, e.g., about 1 mg/kg to about 5 mg/kg, about 2 mg/kg to about 10 mg/kg, about 6 mg/kg to about 40 mg/kg, about 20 mg/kg to about 100 mg/kg, about 50 mg/kg to about 200 mg/kg, about 100 mg/kg to about 400 mg/kg or about 1 mg/kg to about 500 mg/kg are contemplated by the invention.

Viral vectors may be used to administer the nucleic acid encoding an FGF protein to the subject. One type of vector is a “plasmid”, which refers to a circular double stranded DNA loop into which additional DNA segments can be ligated. Another type of vector is a viral vector, wherein additional DNA segments can be ligated into the viral genome (e.g., lentiviral vector). Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions. In one embodiment, the viral vector is a lentivirus expressing an FGF or a shRNA that is directly injected into the adipose tissue of the subject.

A drug delivery matrix may also be used to deliver the FGF receptor agonist (or a cell contacted with an FGF receptor agonist such that UCP1 expression is induced) to the subject. For example, an FGF protein, such as FGF6 is encapsulated into silk scaffolds as described by Jin H. J. et al. (Nature 424:1057-1061, 2003). The silk hydrogel is fashioned using silk fibroin derived from cocoons mixed with polyvinyl alcohol (Wang X. et al., Biomaterials 31:1025-1035, 2010). The silk scaffold is an ideal system for in vivo delivery due to its favorable properties, including controlled release of protein in active form and biocompatibility with minimal immunogenic response. In another embodiment, recombinant FGF6 is loaded into the silk-hydrogel and the targeted release rate and duration are optimized. The prepared hydrogel may be implanted for example, through small incisions into adipose tissue of the subject.

In another aspect, the present invention provides ex vivo methods of treating a subject having a disorder that would benefit from metabolic control. The method comprises administering an FGF-receptive cell contacted with an FGF receptor agonist in which UCP1 expression is induced (e.g., protein or a nucleic acid encoding an FGF protein to the subject), wherein the FGF-receptive cell is administered to the subject, such that the disorder is treated. In one embodiment, prior to administration, the FGF-receptive cell is contacted with an FGF protein or a nucleic acid encoding an FGF protein such as, for example, FGF1, FGF2, FGF4, FGF6, FGF8, FGF9, FGF16, FGF17, FGF18, and FGF20. In one embodiment, the FGF-receptive cell is contacted with a combination of FGF proteins. For example, two or more FGF proteins can be used in combination, specifically, FGF6 and FGF9, FGF6 and FGF2 or FGF9 and FGF2. In another embodiment the combination includes two or more FGF proteins selected from the group consisting of FGF1, FGF2, FGF4, FGF6, FGF8, FGF9, FGF16, FGF17, FGF18, and FGF20. In a specific embodiment, the FGF protein is FGF6. In another specific embodiment, the FGF protein is not FGF21. In one embodiment, the cell is contacted with FGF in vitro prior to administration in the absence of an additional agent selected from the group consisting of an additional growth factor, dexamethasone, and indomethacin

In certain embodiments, the methods of the invention are performed in combination with an adrenergic agonist, including, but not limited to, β-adrenergic agonist, α-adrenergic agonists and mixed agonists. Mixed agonists activate both β-adrenergic receptors and α-adrenergic receptors. Examples of α1 agonists include, for example, amidephrine, anisodamine, anisodine, cirazoline, dipivefrine, dopamine, ephedrine, epinephrine (adrenaline), etilefrine, ethylnorepinephrine, 5-fluoronorepinephrine, 6-fluoronorepinephrine, indanidine, levonordefrin, metaraminol, methoxamine, methyldopa, midodrine, naphazoline, norepinephrine (noradrenaline), octopamine, oxymetazoline, phenylephrine, phenylpropanolamine, pseudoephedrine, synephrine, tetrahydrozoline. Examples of α2 agonists include, for example, amitraz, apraclonidine, brimonidine, cannabivarin, clonidine, cetomidine, cexmedetomidine, cihydroergotamine, cipivefrine, copamine, ephedrine, ergotamine, epinephrine (adrenaline), esproquin, etilefrine, ethylnorepinephrine, 6-fluoronorepinephrine, guanabenz, guanfacine, guanoxabenz, levonordefrin, lofexidine, medetomidine, methyldopa, mivazerol, naphazoline, 4-NEMD, (R)-3-nitrobiphenyline, norepinephrine (noradrenaline), nhenylpropanolamine, piperoxan, pseudoephedrine, rilmenidine, romifidine, talipexole, tetrahydrozoline, tizanidine, tolonidine, trapidil, xylazine, xylometazoline. Examples of β-adrenergic agonists include, for example, abediterol, amibegron, arbutamine, arformoterol, arotinolol, bambuterol, befunolol, bitolterol, bromoacetylalprenololmenthane (BAAM), broxaterol, buphenine, carbuterol, cimaterol, clenbuterol, denopamine, deterenol, dipivefrine, dobutamine, dopamine, dopexamine, ephedrine, epinephrine (adrenaline), etafedrine, etilefrine, ethylnorepinephrine, fenoterol, 2-fluoronorepinephrine, 5-fluoronorepinephrine, formoterol, hexoprenaline, higenamine, indacaterol, isoetarine, isoprenaline (isoproterenol), N-isopropyloctopamine, isoxsuprine, labetalol, levonordefrin, levo salbutamol, mabuterol, methoxyphenamine, methyldopa, norepinephrine (noradrenaline), orciprenaline, oxyfedrine, phenylpropanolamine, pirbuterol, prenalterol, ractopamine, procaterol, pseudoephedrine, reproterol, rimiterol, ritodrine, salbutamol (albuterol), salmeterol, solabegron, terbutaline, tretoquinol, tulobuterol, xamoterol, zilpaterol, zinterol.

In certain embodiments, the methods of the invention are performed in the absence of an adrenergic agonist.

### Fibroblast Growth Factors (FGFs)

Methods and compositions of the invention are based on the discovery, at least in part, that FGFs can induce UCP1 expression in the absence of cell differentiation and can increase energy consumption of mature adipocytes Methods and compositions of the invention are also based, at least in part, on the discovery that FGFs can induce UCP1 expression in the absence of substantial lipid accumulation. Examples of FGFs that may be used in the compositions and methods of the invention are described below. As described above, the term “FGF” is intended to include the protein and nucleic acids encoding the protein, as well as functional fragments thereof (of either the protein or nucleic acid). A functional fragment would retain, for example, the ability of the FGF to induce UCP1. In one embodiment, the methods and compositions of the invention include human FGFs, e.g., administration of human FGF1, human FGF2, human FGF4, human FGF6, human FGF8, human FGF9, human FGF16, human FGF17, human FGF18, and/or human FGF20 to human subject in need thereof in accordance with the methods described herein.

The family of fibroblast growth factors (FGFs) regulates a plethora of developmental processes, including brain patterning, branching morphogenesis and limb development. There are 22 mammalian FGFs grouped into 6 subfamilies by sequence homology and phylogeny. FGF1-subfamily comprises FGF1 and FGF2. FGF4 subfamily comprises FGF4, FGF5 and FGF6. FGF7 subfamily comprises FGF3, FGF7, FGF10 and FGF22. FGF8 subfamily comprises FGF8, FGF17 and FGF18. FGF9 subfamily comprises FGF9, FGF16 and FGF20. FGF19 subfamily comprises FGF19, FGF21 and FGF23. FGF1-10 and FGF15-18 are classically considered as paracrine factors and execute their diverse functions by binding to cell surface FGF receptors (FGFRs) in a heparan sulfate proteoglycans (HSPG)-assisted manner. Owing to their high affinity to HSPG, paracrine FGFs can diffuse only a short distance from their source, thus functioning only locally. By contrast, the subfamily of FGF19, 21, and 23 has been shown to function in an endocrine fashion. Their activities are highly regulated by the co-receptors klotho proteins. It has been shown that FGF21, by acting through the transcription coactivator PGC1α, induces browning of white fat (Fisher F. M. et al., Genes Dev 26:271-281, 2012). In one embodiment, the FGF protein is selected from the group consisting of a fragment thereof, a variant, an analog, a mimetic, a mutein and an FGF protein conjugated to another molecule. Non-limiting examples of FGF proteins include, at least, FGF1, FGF2, FGF4, FGF5, FGF6, FGF8, FGF9, FGF10, FGF16, FGF17, FGF18, FGF20, FGF21 and FGF22. In one embodiment of the present invention, the FGF used in the methods and compositions is selected from the group consisting of FGF1, FGF2, FGF4, FGF6, FGF8, FGF9, FGF16, FGF17, FGF18 and FGF20. Combination of FGFs are also contemplated as part of the invention.

In one embodiment of the invention, the FGF used in the methods and compositions is FGF6, also known as FGF-6, heparin secretory-transforming protein 2, HST-2, HSTF-2, heparin-binding growth factor 6 and HBGF-6. FGF6 belongs to the FGF4 subfamily that consists of FGF4, 5, and 6. FGF6 plays a critical role in muscle development (Armand A. S. et al., Biochim. Biophys. Acta 1763:773-778, 2006) and FGF6 knockout mice have a defect in muscle regeneration (Floss T. et al., Genes Dev. 11:2040-2051, 1997). Like other paracrine FGF proteins, FGF6 binds to the dimerized FGFRs and induces phosphorylation of tyrosine residues in the FGFR, thereby activating the intracellular signaling pathways. The canonical pathways activated by most FGFs are the Ras-mitogen-activated kinase (MAPK) and the phosphoinositide 3-kinase (PI3K)-Akt pathways (Jin M. et al., Cell Biol. Int. 36:691-696, 2012). The sequence of a human FGF6 mRNA sequence can be found at, for example, GenBank Accession No. GI:10337586 (NM_020996.1; SEQ ID NO:1). The sequence of a human FGF6 polypeptide sequence can be found at, for example, GenBank Accession No. GI:15147343 (NP_066276.2; SEQ ID NO: 2). The sequence of murine FGF6 mRNA sequence can be found at, for example, GenBank Accession No. GI:112363075 (NM_010204.1; SEQ ID NO: 3). The sequence of murine FGF6 polypeptide sequence can be found at, for example, GenBank Accession No. GI:112363076 (NP_034334.1; SEQ ID NO: 4).

In one embodiment of the invention, the FGF used in the methods and compositions is FGF1, also known as endothelial cell growth factor, ECGF, heparin-binding growth factor 1 and HBGF-1. The sequence of a human FGF1 mRNA sequence can be found at, for example, GenBank Accession No. GI:380748935 (NM_000800.4; SEQ ID NO:9). The sequence of a human FGF1 polypeptide sequence can be found at, for example, GenBank Accession No. GI:4503697 (NP_000791.1; SEQ ID NO:10). The sequence of murine FGF1 mRNA sequence can be found at, for example, GenBank Accession No. GI:122937366 (NM_010197.3; SEQ ID NO:11). The sequence of murine FGF1 polypeptide sequence can be found at, for example, GenBank Accession No. GI:6753850 (NP_0034327.1; SEQ ID NO:12).

In one embodiment of the invention, the FGF used in the methods and compositions is FGF2, also known as heparin-binding growth factor 2 and HBGF-2. FGF2 induces angiogenesis, fibroblast proliferation, cell differentiation, neurogenesis and vascular remodeling. The sequence of a human FGF2 mRNA sequence can be found at, for example, GenBank Accession No. GI:153285460 (NM_002006.4; SEQ ID NO:13). The sequence of a human FGF2 polypeptide sequence can be found at, for example, GenBank Accession No. GI:153285461 (NP_001997.5; SEQ ID NO:14). The sequence of murine FGF2 mRNA sequence can be found at, for example, GenBank Accession No. GI:159032535 (NM_008006.2; SEQ ID NO:15). The sequence of murine FGF2 polypeptide sequence can be found at, for example, GenBank Accession No. GI:7106315 (NP_032032.1; SEQ ID NO:16).

In one embodiment of the invention, the FGF used in the methods and compositions is FGF4, also known as heparin secretory transforming protein 1, HST-1, heparin-binding growth factor 4 and HBGF-4. The sequence of a human FGF4 mRNA sequence can be found at, for example, GenBank Accession No. GI:196049393 (NM_002007.2; SEQ ID NO:17). The sequence of a human FGF4 polypeptide sequence can be found at, for example, GenBank Accession No. GI:4503701 (NP_001998.1; SEQ ID NO:18). The sequence of murine FGF4 mRNA sequence can be found at, for example, GenBank Accession No. GI:158508679 (NM_010202.5; SEQ ID NO:19). The sequence of murine FGF4 polypeptide sequence can be found at, for example, GenBank Accession No. GI:66955870 (NP_034332.2; SEQ ID NO:20).

In one embodiment of the invention, the FGF used in the methods and compositions is FGF5, also known as heparin-binding growth factor 5 and HBGF-5. The sequence of a human FGF5 mRNA sequence can be found at, for example, GenBank Accession No. GI:73486654 (NM_004464.3; SEQ ID NO:21). The sequence of a human FGF5 polypeptide sequence can be found at, for example, GenBank Accession No. GI:73486655 (NP_004455.2; SEQ ID NO:22). The sequence of murine FGF5 mRNA sequence can be found at, for example, GenBank Accession No. GI:145966820 (NM_010203.4; SEQ ID NO:23). The sequence of murine FGF5 polypeptide sequence can be found at, for example, GenBank Accession No. GI:6753854 (NP_034333.1; SEQ ID NO:24).

In one embodiment of the invention, the FGF used in the methods and compositions is FGF8, also known as andergen-induced growth factor, AIGF, heparin-binding growth factor 8 and HBGF-8. The sequence of a human FGF8 mRNA sequence can be found at, for example, GenBank Accession No. GI:329755302 (NM_001206389.1; SEQ ID NO:25). The sequence of a human FGF8 polypeptide sequence can be found at, for example, GenBank Accession No. GI:329755303 (NP_001193318.1; SEQ ID NO:26). The sequence of murine FGF8 mRNA sequence can be found at, for example, GenBank Accession No. GI:261599073 (NM_001166361.1; SEQ ID NO:27). The sequence of murine FGF8 polypeptide sequence can be found at, for example, GenBank Accession No. GI:261599074 (NP_001159833.1; SEQ ID NO:28).

In one embodiment of the invention, the FGF used in the methods and compositions is FGF9, also known as glia-activating factor, GAF, heparin-binding growth factor 9 and HBGF-9. FGF9 induces angiogenesis, vascularization, osteoblast differentiation, and chondrocyte differentiation. The sequence of a human FGF9 mRNA sequence can be found at, for example, GenBank Accession No. GI: GI:209529671 (NM_002010.2; SEQ ID NO:5). The sequence of a human FGF9 polypeptide sequence can be found at, for example, GenBank Accession No. GI:4503707 (NP_002001.1; SEQ ID NO:6). The sequence of murine FGF9 mRNA sequence can be found at, for example, GenBank Accession No. GI:261824046 (NM_013518.4; SEQ ID NO:7). The sequence of murine FGF9 polypeptide sequence can be found at, for example, GenBank Accession No. GI:110625633 (NP_038546.2; SEQ ID NO:8).

In one embodiment of the invention, the FGF used in the methods and compositions is FGF10, also known as keratinocyte growth factor 2. The sequence of a human FGF10 mRNA sequence can be found at, for example, GenBank Accession No. GI:4758359 (NM_004465.1; SEQ ID NO:29). The sequence of a human FGF10 polypeptide sequence can be found at, for example, GenBank Accession No. GI:4758360 (NP_004456.1; SEQ ID NO:30). The sequence of murine FGF10 mRNA sequence can be found at, for example, GenBank Accession No. GI:226823275 (NM_008002.4; SEQ ID NO:31). The sequence of murine FGF10 polypeptide sequence can be found at, for example, GenBank Accession No. GI:7106313 (NP_032028.1; SEQ ID NO:32).

In one embodiment of the invention, the FGF used in the methods and compositions is FGF16. The sequence of a human FGF16 mRNA sequence can be found at, for example, GenBank Accession No. GI:4503690 (NM_003868.1; SEQ ID NO:33). The sequence of a human FGF16 polypeptide sequence can be found at, for example, GenBank Accession No. GI:4503691 (NP_003859.1; SEQ ID NO:34). The sequence of murine FGF16 mRNA sequence can be found at, for example, GenBank Accession No. GI:126116562 (NM_030614.2; SEQ ID NO:35). The sequence of murine FGF16 polypeptide sequence can be found at, for example, GenBank Accession No. GI:126116563 (NP_085117.2; SEQ ID NO:36).

In one embodiment of the invention, the FGF used in the methods and compositions is FGF17. The sequence of a human FGF17 mRNA sequence can be found at, for example, GenBank Accession No. GI:61743927 (NM_003867.2; SEQ ID NO:37). The sequence of a human FGF17 polypeptide sequence can be found at, for example, GenBank Accession No. GI:4503693 (NP_003858.1; SEQ ID NO:38). The sequence of murine FGF17 mRNA sequence can be found at, for example, GenBank Accession No. GI:145966703 (NM_008004.4; SEQ ID NO:39). The sequence of murine FGF17 polypeptide sequence can be found at, for example, GenBank Accession No. GI:6679779 (NP_032030.1; SEQ ID NO:40).

In one embodiment of the invention, the FGF used in the methods and compositions is FGF18. The sequence of a human FGF18 mRNA sequence can be found at, for example, GenBank Accession No. GI:300796572 (NM_003862.2; SEQ ID NO:41). The sequence of a human FGF18 polypeptide sequence can be found at, for example, GenBank Accession No. GI:4503695 (NP_003853.1; SEQ ID NO:42). The sequence of murine FGF18 mRNA sequence can be found at, for example, GenBank Accession No. GI:6679780 (NM_008005.1; SEQ ID NO:43). The sequence of murine FGF18 polypeptide sequence can be found at, for example, GenBank Accession No. GI:6679781 (NP_032031.1; SEQ ID NO:44).

In one embodiment of the invention, the FGF used in the methods and compositions is FGF20. The sequence of a human FGF20 mRNA sequence can be found at, for example, GenBank Accession No. GI:262263324 (NM_019851.2; SEQ ID NO:45). The sequence of a human FGF20 polypeptide sequence can be found at, for example, GenBank Accession No. GI:9789947 (NP_062825.1; SEQ ID NO:46). The sequence of murine FGF20 mRNA sequence can be found at, for example, GenBank Accession No. GI:255683332 (NM_030610.2; SEQ ID NO:47). The sequence of murine FGF20 polypeptide sequence can be found at, for example, GenBank Accession No. GI:255683333 (NP_085113.2; SEQ ID NO:48).

In one embodiment of the invention, the FGF protein used in the methods and compositions is FGF21. The sequence of a human FGF21 mRNA sequence can be found at, for example, GenBank Accession No. GI:68215584 (NM_019113.2; SEQ ID NO:53). The sequence of a human FGF21 polypeptide sequence can be found at, for example, GenBank Accession No. GI:9506597 (NP_061986.1; SEQ ID NO:54). The sequence of murine FGF21 mRNA sequence can be found at, for example, GenBank Accession No. GI:146134956 (NM_020013.4; SEQ ID NO:55). The sequence of murine FGF21 polypeptide sequence can be found at, for example, GenBank Accession No. GI:9910218 (NP_064397.1; SEQ ID NO:56).

In one embodiment of the invention, the FGF used in the methods and compositions is FGF22. The sequence of a human FGF22 mRNA sequence can be found at, for example, GenBank Accession No. GI:10190671 (NM_020637.1; SEQ ID NO:49). The sequence of a human FGF22 polypeptide sequence can be found at, for example, GenBank Accession No. GI:10190672 (NP_065688.1; SEQ ID NO:50). The sequence of murine FGF22 mRNA sequence can be found at, for example, GenBank Accession No. GI:12963626 (NM_023304.1; SEQ ID NO:51). The sequence of murine FGF22 polypeptide sequence can be found at, for example, GenBank Accession No. GI:12963627 (NP_075793.1; SEQ ID NO:52).

The present invention also includes use of variants of FGF proteins. Such variants have an altered amino acid sequences that can function as agonists or mimetics. Variants can be generated by mutagenesis, e.g., discrete point mutation or truncation. An agonist can retain substantially the same, or a subset, of the biological activities of the naturally occurring form of the protein.

Accordingly, another aspect of the invention pertains to use of variant FGF proteins (or nucleic acid molecules encoding a variant protein) that contain changes in amino acid residues that are not essential for activity, e.g., wherein the variant FGF protein retains the ability to activate the FGF receptor and induce UCP1 expression in the cell. Such variant proteins differ in amino acid sequence from the naturally-occurring proteins, yet retain biological activity. In one embodiment, such a variant protein has an amino acid sequence that is at least about 40% identical, 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of an FGF protein recited above e.g., FGF1 (SEQ ID NO:10), FGF2 (SEQ ID NO:14), FGF4 (SEQ ID NO:18), FGF6 (SEQ ID NO:2), FGF8 (SEQ ID NO:26), FGF9 (SEQ ID NO:6), FGF16 (SEQ ID NO:34), FGF17 (SEQ ID NO:38), FGF18 (SEQ ID NO:42), and FGF20 (SEQ ID NO:46).

Variants of an FGF protein that function as agonists (mimetics) can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the marker proteins from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

In a further embodiment, the invention also may be practiced using a mimetic of an FGF protein.

The invention also provides chimeric or fusion proteins comprising an FGF protein, e.g., FGF1, FGF2, FGF4, FGF6, FGF8, FGF9, FGF16, FGF17, FGF18 and FGF20, or a functional fragment thereof. As used herein, a “chimeric protein” or “fusion protein” comprises all or part (preferably a biologically active part) of an FGF protein operably linked to a heterologous polypeptide (i.e., a polypeptide other than FGF protein). Within the fusion protein, the term “operably linked” is intended to indicate that the FGF protein or segment thereof and the heterologous polypeptide are fused in-frame to each other. The heterologous polypeptide can be fused to the amino-terminus or the carboxyl-terminus of the FGF protein or functional fragment thereof.

In one embodiment of the invention, the methods and compositions use chimeric or fusion proteins comprising an FGF protein, e.g., FGF1, FGF2, FGF4, FGF6, FGF8, FGF9, FGF16, FGF17, FGF18 and FGF20, or a functional fragment (or portion) thereof. Biologically active portions of an FGF protein, e.g., FGF1, FGF2, FGF4, FGF6, FGF8, FGF9, FGF16, FGF17, FGF18 and FGF20, are also included within the scope of the present invention. Such biologically active portions include polypeptides comprising amino acid sequences sufficiently identical to or derived from the amino acid sequence of an FGF protein, e.g., FGF1, FGF2, FGF4, FGF6, FGF8, FGF9, FGF16, FGF17, FGF18 and FGF20, which include fewer amino acids than the full length protein, and exhibit at least one activity of the corresponding full-length protein. Typically, biologically active portions comprise a domain or motif with at least one activity of the corresponding full-length protein, e.g., ability to induce UCP1 expression. A biologically active portion of a protein for use in the methods of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of the protein.

Suitable FGF proteins, as described above, for use in the methods of the present invention may be either naturally occurring (native) or genetically engineered. For example, suitable FGF proteins may be obtained by, for example, use of an appropriate purification scheme using standard protein purification techniques. Alternatively, recombinant DNA techniques may be used to produce a FGF protein comprising the whole or a segment of the protein (a functional fragment of the protein). For example, recombinant DNA techniques may be used to clone a nucleotide sequence encoding a segment or the whole protein into a vector (such as an expression vector) and transform a cell for production of the protein. An FGF protein comprising the whole or a segment of the protein may also be synthesized chemically using standard peptide synthesis techniques.

RNA or DNA encoding the FGF proteins may be readily isolated, amplified, and/or sequenced using conventional procedures (e.g., by using oligonucleotide probes that are capable of binding specifically to the relevant genes, as described in, for example, Innis et al. in PCR Protocols. A Guide to Methods and Applications, Academic (1990), and Sanger et al., Proc Natl Acad Sci USA 74:5463 (1977)). A nucleic acid molecule so amplified may be cloned into an appropriate vector and characterized by DNA sequence analysis. Furthermore, nucleotides corresponding to all or a portion of an isolated nucleic acid molecule for use in the methods of the invention can be prepared by standard synthetic techniques, e.g., using an automated DNA synthesizer.

In one embodiment, an isolated nucleic acid molecule for use in the methods of the invention comprises a nucleic acid molecule which has a nucleotide sequence complementary to the nucleotide sequence of a nucleic acid molecule encoding, an FGF protein, for example, FGF1, FGF2, FGF4, FGF6, FGF8, FGF9, FGF16, FGF17, FGF18 or FGF20. A nucleic acid molecule which is complementary to a given nucleotide sequence is one which is sufficiently complementary to the given nucleotide sequence that it can hybridize to the given nucleotide sequence thereby forming a stable duplex.

The invention further encompasses nucleic acid molecules that differ, due to degeneracy of the genetic code, from the nucleotide sequence of nucleic acid molecules encoding an FGF protein, e.g., FGF1, FGF2, FGF4, FGF6, FGF8, FGF9, FGF16, FGF17, FGF18 and FGF20, and thus encode the same protein. It will be appreciated by those skilled in the art that DNA sequence polymorphisms that lead to changes in the amino acid sequence can exist within a population. Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

Accordingly, in one embodiment a nucleic acid molecule suitable for use in the methods of the invention is at least about 40% identical, about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or about 99% identical to the nucleotide sequence of an FGF protein, e.g., FGF1, FGF2, FGF4, FGF6, FGF8, FGF9, FGF16, FGF17, FGF18 and FGF20.

In addition to naturally occurring allelic variants of a nucleic acid molecule of the invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species may be essential for activity and thus would not be likely targets for alteration.

FGF protein for use in the invention may be made according to methods know in the art. The recombinant vectors can comprise a nucleic acid encoding an FGF in a form suitable for expression of the nucleic acid in a host cell. In some embodiments, this means that the recombinant vectors may include one or more regulatory sequences, selected on the basis of the host cells to be used for expression, which is operably linked to the nucleic acid sequence to be expressed (i.e., a recombinant expression vector). Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Enzymology: Gene Expression Technology vol. 185, Academic Press, San Diego, Calif. (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide, or functional fragment thereof, in prokaryotic (e.g., E. coli) or eukaryotic cells (e.g., insect cells {using baculovirus expression vectors}, yeast cells or mammalian cells). Suitable host cells are discussed further in Goeddel, supra, and include, for example, E. coli cells, Bacillus cells, Saccharomyces cells, Pochia cells, NS0 cells, COS cells, Chinese hamster ovary (CHO) cells or myeloma cells. The RNA or DNA also may be modified, for example, by substituting bases to optimize for codon usage in a particular host or by covalently joining to the coding sequence of a heterologous polypeptide. Such an approach would be the basis for developing a subunit vaccine. Alternatively, the recombinant expression vector can be transcribed and translated in vitro.

Another aspect of the invention pertains to host cells into which a recombinant vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

A signal sequence can be used to facilitate secretion and isolation of FGF proteins. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to FGF proteins, fusion proteins or segments thereof having a signal sequence, as well as to such proteins from which the signal sequence has been proteolytically cleaved (i.e., the cleavage products). In one embodiment, a nucleic acid sequence encoding a signal sequence can be operably linked in an expression vector to a nucleic acid molecule encoding a protein of interest, such as an FGF protein, e.g., FGF1, FGF2, FGF4, FGF6, FGF8, FGF9, FGF16, FGF17, FGF18 and FGF20, or a segment thereof. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a poly-histidine tag, a strep-tag, a FLAG-tag, a GST domain, etc.

The invention further contemplates methods and compositions comprising an anti-FGF receptor antibody, or antigen binding portion thereof, which activates an FGF receptor and can induce expression of UCP1 in an FGF receptive cell. In one embodiment, the anti-FGF receptor antibody, or antigen binding portion thereof, increases UCP1 mRNA expression and/or UCP1 protein expression. In another embodiment, by binding to the FGF receptor, the anti-FGF receptor antibody, or antigen binding portion thereof, increases tyrosine kinase activity. The increase of tyrosine kinase activity may be determined, e.g. by immunoprecipitation of target proteins and subsequent determination using suitable anti-phosphotyrosine antibodies.

Agonist anti-FGF receptor antibodies, such as agonist anti-FGFR1 antibodies, may be identified, screened for (e.g., using phage display), or characterized for their physical/chemical properties and/or biological activities by various assays known in the art (see, for example, Antibodies: A Laboratory Manual, Second edition, Greenfield. ed., 2014). Assays, for example, described in the Examples may be used to identify antibodies having advantageous properties, such as the ability to increase energy expenditure in the absence of adipocyte differentiation. In one aspect, an anti-FGF receptor antibody is tested for its antigen binding activity, e.g., by known methods such as ELISA, Western blot, etc.

Following identification of the antigen of the antibody e.g., ability to bind FGFR1, the activity of the antibody may be tested. In one aspect, assays are provided for identifying anti-FGFR antibodies, e.g., FGFR1, thereof having agonistic activity. For example, biological activity may include the ability to activate signal transduction of particular pathways which can be measured, e.g., by determining levels of phospho-FRS2a, phospho-MEK, phospho-phospho-STAT3 or using the GAL-Elk1-based luciferase assays described herein (see also, e.g., Wu et al. J. Biol. Chem. 5; 282(40):29069-72 (2007) and Wu et al. PLoS One 18; 6(3):e17868 (2011).

Following screening and sequencing, antibodies may be produced using recombinant methods and compositions, e.g., as described in U.S. Pat. No. 4,816,567, incorporated by reference herein. An isolated nucleic acid encoding, for example, an anti-FGFR1 antibody is used to transform host cells for expression. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VU of the antibody. In one embodiment, the host cell is eukaryotic, e.g. a Chinese Hamster Ovary (CHO) cell or lymphoid cell (e.g., Y0, NS0, Sp20 cell).

For recombinant production of an anti-FGF receptor, e.g., FGFR I, antibody, nucleic acid encoding an antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HFLA); canine kidney cells (MDCK; buffalo rat liver cells (BRL 3A); human lung cells (W138); human liver cells (Her; G2); mouse mammary tumor (MMT 060562); TR1 cells, as described, e.g., in Mather et al., Annals N.Y. Acad. Sci, 383:44-68 (1982); MRC 5 cells; and FS4 cells, Other useful mammalian host cell lines include Chinese hamster ovary (CHO) cells, including DHFR.sup.-CHO cells (Urlaub et al., Proc. Nati, Acad. Sri. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

In one embodiment, the anti-FGF receptor antibody, or antigen binding portion thereof, binds to and activates FGF Receptor 1 (FGFR1). Such FGFR1 agonist antibodies can induce expression of UCP1 in a FGF receptive cell. Examples of FGFR1 agonist antibodies are described in US Patent Application Publication No. US 20120294861 (Genentech), the antibody sequences of which are incorporated by reference herein as well as the methods for screening for anti-FGFR1 agonist antibodies.

### Pharmaceutical Compositions

Therapeutic formulations comprising an FGF receptor agonist (e.g., an FGF protein, FGF mimetic, nucleic acids encoding an FGF protein, such as FGF1, FGF2, FGF4, FGF6, FGF8, FGF9, FGF16, FGF17, FGF18 and FGF20, or an anti-FGF receptor agonist antibody) of the present invention may be prepared for storage by mixing the protein or nucleic acid having the desired degree of purity with optional physiologically acceptable carriers, excipients or stabilizers (Remington's Pharmaceutical Sciences 16th edition, Osol, A. Ed. (1980)), in the form of aqueous solutions, lyophilized or other dried formulations. Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as Tween™, Pluronics™ or polyethylene glycol (PEG).

The formulation herein may also contain more than one active compound as necessary for the particular indication being treated (e.g., a disease that would benefit from glucose control, a disease that would benefit from weight control, a disease that would benefit from appetite control), preferably those with complementary activities that do not adversely affect each other. Such molecules are suitably present in combination in amounts that are effective for the purpose intended. In one embodiment, the active compound is a diabetic therapy. In another embodiment, the active compound is an HMG-CoA reductase inhibitor.

The active ingredients (e.g., an FGF protein, FGF mimetic, a nucleic acid encoding an FGF protein, or an anti-FGF receptor agonist antibody) may also be packaged in a microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Sustained-release preparations of the FGF receptor agonist (e.g., an FGF protein, FGF mimetic, a nucleic acid encoding an FGF protein, or an anti-FGF receptor agonist antibody), may be prepared. Suitable examples of sustained-release preparations include semipermeable matrices of solid hydrophobic polymers containing the immunoglobulin of the invention, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

Generally, the ingredients of compositions are supplied either separately or mixed together in unit dosage form, for example, as a dry lyophilized powder or water free concentrate in a hermetically sealed container such as an ampoule or sachet indicating the quantity of active agent. Where the mode of administration is infusion, composition can be dispensed with an infusion bottle containing sterile pharmaceutical grade water or saline. Where the mode of administration is by injection, an ampoule of sterile water for injection or saline can be provided so that the ingredients may be mixed prior to administration. In an alternative embodiment, one or more of the pharmaceutical compositions of the invention is supplied in liquid form in a hermetically sealed container indicating the quantity and concentration of the agent.

The FGF receptor agonist (e.g., an FGF protein, FGF mimetic, a nucleic acid encoding an FGF protein, or an anti-FGF receptor agonist antibody) can be incorporated into a pharmaceutical composition suitable for parenteral administration, typically prepared as an injectable solution. The injectable solution can be composed of either a liquid or lyophilized dosage form in a flint or amber vial, ampule or pre-filled syringe. The liquid or lyophilized dosage may further comprise a buffer (e.g., L-histidine, sodium succinate, sodium citrate, sodium phosphate or potassium phosphate, sodium chloride), a cryoprotectant (e.g., sucrose trehalose or lactose, a bulking agent (e.g., mannitol), a stabilizer (e.g., L-Methionine, glycine, arginine), an adjuvant (hyaluronidase).

The compositions of this invention may be in a variety of forms. These include, for example, liquid, semi-solid and solid dosage forms, such as liquid solutions (e.g., injectable and infusible solutions), microemulsion, dispersions, liposomes or suspensions, tablets, pills, powders, liposomes and suppositories. The preferred form depends on the intended mode of administration and therapeutic application. Typical modes of administration include parenteral (e.g., intravenous, subcutaneous, intraperitoneal, intramuscular) injection or oral administration. In a preferred embodiment, the FGF receptor agonist (e.g., an FGF protein, FGF mimetic, a nucleic acid encoding an FGF protein, or an anti-FGF receptor agonist antibody) is administered by injection. In another embodiment, the injection is subcutaneous. In a particular embodiment, the administration is into adipose tissue

Pharmaceutical compositions comprising an FGF receptor agonist (e.g., an FGF protein, FGF mimetic, a nucleic acid encoding an FGF protein, or an anti-FGF receptor agonist antibody) may be formulated for administration to a particular tissue. For example, in certain embodiments, it may be desirable to administer the FGF receptor agonist into adipose tissue, either in a diffuse fashion or targeted to a site (e.g., subcutaneous adipose tissue).

In another aspect, the invention provides pharmaceutical compositions that utilize cells in various methods for treatment of diseases that would benefit from glucose control, weight control and or appetite control. Certain embodiments encompass pharmaceutical compositions comprising live cells (e.g., an FGF-receptive cell contacted with an FGF receptor agonist such that UCP1 expression is induced). The pharmaceutical composition may further comprise other active agents, such as anti-inflammatory agents, anti-apoptotic agents, antioxidants or growth factors.

Examples of other components that may be added to cell pharmaceutical compositions include, but are not limited to: (1) selected extracellular matrix components, such as one or more types of collagen known in the art, and/or growth factors, platelet-rich plasma, and drugs (alternatively, FGF-receptive cells may be genetically engineered to express and produce growth factors); (2) anti-apoptotic agents (e.g., erythropoietin (EPO), EPO mimetibody, thrombopoietin, insulin-like growth factor (IGF)-I, hepatocyte growth factor, caspase inhibitors); (3) anti-inflammatory compounds (e.g., p38 MAP kinase inhibitors, TGF-beta inhibitors, statins, IL-6 and IL-1 inhibitors, PEMIROLAST, TRANILAST, REMICADE, SIROLIMUS, and non-steroidal anti-inflammatory drugs (NSAIDS) (such as Tepoxalin, Tolmetin, and Suprofen); (4) immunosuppressive or immunomodulatory agents, such as calcineurin inhibitors, mTOR inhibitors, antiproliferatives, corticosteroids and various antibodies; (5) antioxidants such as probucol, vitamins C and E, coenzyme Q-10, glutathione, L-cysteine and N-acetylcysteine; (6) local anesthetics; (7) diabetic therapies; and (8) HMG-CoA reductase inhibitors, to name a few.

Pharmaceutical compositions of the invention comprise an FGF-receptive cell contacted with an FGF receptor agonist (e.g., an FGF protein, FGF mimetic, a nucleic acid encoding an FGF protein, or an anti-FGF receptor agonist antibody) such that UCP1 expression is induced, or components or products thereof, formulated with a pharmaceutically acceptable carrier or medium. Suitable pharmaceutically acceptable carriers include water, salt solution (such as Ringer's solution), alcohols, oils, gelatins, and carbohydrates, such as lactose, amylose, or starch, fatty acid esters, hydroxymethylcellulose, and polyvinyl pyrrolidine. Such preparations can be sterilized, and if desired, mixed with auxiliary agents such as lubricants, preservatives, stabilizers, wetting agents, emulsifiers, salts for influencing osmotic pressure, buffers, and coloring. Pharmaceutical carriers suitable for use in the present invention are known in the art and are described, for example, in Pharmaceutical Sciences (17th Ed., Mack Pub. Co., Easton, Pa.) and WO 96/05309.

Pharmaceutical compositions comprising an FGF-receptive cell contacted with an FGF receptor agonist (e.g., an FGF protein, FGF mimetic, a nucleic acid encoding an FGF protein, or an anti-FGF receptor agonist antibody) such that UCP1 expression is induced, are typically formulated as liquids, semisolids (e.g., gels) or solids (e.g., matrices, scaffolds). Liquid compositions are formulated for administration by any acceptable route known in the art to achieve delivery of live cells to the target tissues. Typically, these include injection or infusion into adipose tissue, either in a diffuse fashion or targeted to a site (e.g., subcutaneous adipose tissue).

Pharmaceutical compositions comprising an FGF-receptive cell contacted with an FGF receptor agonist (e.g., protein or a nucleic acid encoding an FGF protein) in a semi-solid or solid carrier are typically formulated for surgical implantation. It will be appreciated that liquid compositions also may be administered by surgical procedures. In particular embodiments, semi-solid or solid pharmaceutical compositions may comprise semi-permeable gels, lattices, cellular scaffolds and the like, which may be non-biodegradable or biodegradable.

In other embodiments, different varieties of degradable gels and networks are utilized for the pharmaceutical compositions of the invention. For example, degradable materials particularly suitable for sustained release formulations include biocompatible polymers, such as poly(lactic acid), poly(lactic-co-glycolic acid), methylcellulose, hyaluronic acid, collagen, and the like. The structure, selection and use of degradable polymers in drug delivery vehicles have been reviewed in several publications, including, A. Domb et al., 1992, Polymers for Advanced Technologies 3:279.

In one embodiment, the methods described herein are done in a human. In a further embodiment, the methods described herein are not performed on a mouse or other non-human animal.

The contents of all references, patents and published patent applications cited throughout this application are incorporated herein by reference

This invention is further illustrated by the following examples, which should not be construed as limiting.

### EXAMPLES

### Methods of the Examples

The following methods were used in the examples below unless otherwise specified.

### RNA Isolation and Quantification of Gene Expression by Q-RT-PCR (QPCR)

Total RNA was isolated with QIAzol lysis reagent (Qiagen, Valencia, Calif.) and purified by RNeasy Mini columns (Qiagen) following the manufacture's instructions. cDNA was prepared from total RNA using the Advantage RT-PCR kit (BD Biosciences, Palo Alto, Calif.) according to manufacturer's instructions. Diluted cDNA was used in a PCR reaction with SYBR Green Master Mix (Applied Biosystems, Foster City, Calif.) and primers specifically designed for detection of the gene of interest. PCR reactions were run in duplicate for each sample and quantitated in the ABI Sequence Detection System (Applied Biosystems). Data were expressed as arbitrary units after normalization to levels of expression of internal controls for each sample. Sequences of primers for specific genes are each obtained from the published literature or designed using publically available gene sequence databases. Unless otherwise indicated, gene expression data described in the examples below was obtained using QPCR.

### Oil Red O Staining

Cell culture dishes were washed twice with phosphate-buffered saline and fixed with 10% buffered formalin overnight at 4° C. Cells were then stained for 2 hours at room temperature with a filtered oil red O solution (0.5% oil red O in isopropyl alcohol), washed twice with distilled water, and visualized.

### Western Blot Analysis

Cells were harvested in lysis buffer (50 mM HEPES, 137 mM NaCl, 1 mM MgCl₂, 1 mM CaCl₂, 10 mM Na₂P₂O₇, 10 mM NaF, 2 mM EDTA, 10% glycerol, 1% Igepal CA-630, 2 mM vanadate, 10 μg/ml of leupeptin, 10 μg/ml of aprotinin, 2 mM phenylmethylsulfonyl fluoride; pH 7.4). After lysis, lysates were clarified by centrifugation at 12,000×g for 20 min at 4° C., the amount of protein in the supernatants was determined by the Bradford Protein Assay (Bio-Rad Laboratories, Hercules, Calif.). Proteins were directly solubilized in Laemmli sample buffer. Equal amounts of lysates were separated by sodium dodecyl sulfate-polyacrylamide gel electrophoresis and transferred to Immobolin-P membranes. Membranes were blocked overnight at 4° C. and incubated with the indicated antibody for 2 hours at room temperature. Specifically bound primary antibodies were detected with peroxidase-coupled secondary antibody and enhanced chemiluminescence (ECL, Amersham Biosciences, Piscataway, N.J.).

### Isolation of Stromo-Vascular Fractions (SVF) and In Vitro Differentiation

Eight 6-week old C57BL/6 male mice were sacrificed. Interscapular BAT and axillary subcutaneous WAT were removed, minced and digested with 1 mg/ml collagenase for 45 minutes at 37° C. in DMEM/F12 media, containing 1% BSA and antibiotics. Digested tissues were filtered through sterile 150 μm nylon mesh and centrifuged at 250×g for 5 minutes. The floating fractions consisting of adipocytes were discarded and the pellets representing the SVF were then resuspended in erythrocyte lysis buffer (154 mM NH₄Cl, 10 mM KHCO₃, 0.1 mM EDTA) for 10 minutes to remove red blood cells. The cells were further centrifuged at 500×g for 5 minutes, plated at 8×10⁵ cells/well of a 24-well plate and grown at 37° C. in DMEM/F12 supplemented with 10% FBS at 37° C.

### Histology and Immunohistochemistry

Tissues were fixed in 10% formalin and paraffin-embedded. Multiple sections were prepared and stained with H&E for general morphological observation. UCP1 immunohistochemistry of tissue from implanted cells was performed using polyclonal anti-mouse UCP1 antibody (Chemicon International Inc., Temecula, Calif.) at a 1:50 dilution and the Dako Envision Doublestain System (Dako, Carpinteria, Calif.) following the manufacture's instruction. Slides were counterstained with Hematoxylin.

### FGF and BMP7 Proteins

Recombinant human FGFs were purchased from R&D Systems (Minneapolis, Minn.) and reconstituted in buffer recommended by the manufacturer. rhBMP7 were provided by Stryker Regenerative Medicine.

### Cell Culture

WT brown preadipocyte cell lines derived from newborn wild-type mice were generated as described previously (Klein J. et al., J. Biol. Chem. 274:34795-34802, 1999; Fasshauer M. et al., J. Biol. Chem. 275:25494-25501, 2000; Fasshauer M. et al., Mol. Cell. Biol. 21:319-329, 2001; Tseng Y. H. et al., J. Biol. Chem. 277:31601-31611, 2002). 3T3-F442A and C2C12 cells were purchased from ATCC. All cell lines used in this study were maintained in Dulbecco's modified Earle's media (DMEM) 10% FBS at 37° C. in 5% CO₂ environment unless otherwise specified.

### Adipocyte Differentiation

To induce adipocyte differentiation by BMP7, in the absence of induction media, both WT brown preadipocytes were grown in regular growth media supplemented with the combination of BMP7 (3.3 nM), insulin (20 nM) and triiodothyronine (T3) (1 nM) or vehicle for 7-8 days.

Adipocytes were differentiated using induction media by growing cells to confluence in growth media supplemented with 20 nM insulin and 1 nM triiodothyronine (T3) for 2-3 days, followed by treatment of the confluent cells for 48 hours with media supplemented with 20 nM insulin, 1 nM T3, 0.5 mM isobutylmethylxanthine (IBMX), and 0.5 μM dexamethasone (i.e., induction media). Cells were placed back in growth media supplemented with insulin and T3, which was changed every second day. After four to five additional days in growth media, cells exhibited a fully differentiated phenotype with massive lipid accumulation.

### Seahorse Bioanalyzer

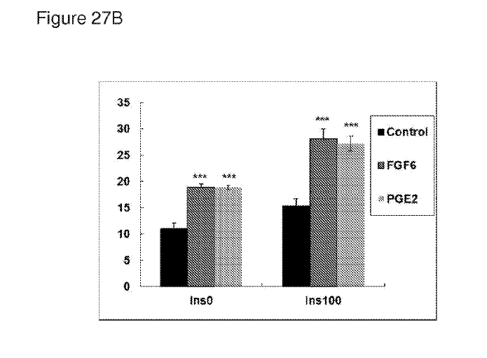

The Seahorse XF24 (Seahorse Bioscience, http://www.seahorsebio.com/) was utilized for respirometry to measure oxygen consumption rates (OCR; indicating oxidative phosphorylation) and extracellular acidification rates (ECAR; indicating extracellular pH) in mature brown adipocytes. The cell plates and assay cartridges for the Seahorse XF24 have four ports allowing for drug delivery to individual wells during measurement of the metabolic parameters. For a typical bioenergetic profile, cells are first treated with oligomycin to block ATP synthase, FCCP as an uncoupler and Rotenone to block Complex 1 of the ETC (all from Sigma). Example 2 provides additional details regarding the general method. Data provided in the Figures (e.g., FIG. 27) based on the Seahorse Bioanalyzer includes a bottom horizontal line which represents the baseline of the mechanics of the machine and is not related to the experiment.

### Plasmids, Cloning, Transfection and Transduction

FGF6 lentiviral vector was generated by cloning mouse FGF6 cDNA into a lentiviral vector. Lentiviruses were obtained by transfecting 293T cells with lentiviral vectors. Viral supernatant was filtered through 0.45 um filter before applying to cells. Transduction was accomplished by incubating cells with virus supernatant. Stable cells were established by drug selection. The lentiviral vector contains a drug resistant gene for selection.

### Isolation and Culture of Primary Human White and Brown Fat Progenitors

Primary stromal-vascular fraction (SVF) from human neck fat was isolated as described previously (Cannon and Nedergaard, Physiol Rev, 84: 277-359, 2004). Briefly, freshly resected superficial fat (pooled subcutaneous and subplatysmal) and fat located in the deeper neck regions (pooled carotid sheath, longus colli and prevertebral) were collected, minced and digested using collagenase 1 (2 mg/mL in PBS with the addition of 3.5% BSA; Worthington Biochemical Corporation, Lakewood, N.J.), and the SVF was isolated. SVF cells were plated and grown in high glucose Dulbecco's modified Eagle's medium (DMEM/H) supplemented with 10% (v/v) fetal bovine serum (FBS) and 1% penicillin/streptomycin. For adipocyte differentiation, cells were grown to confluent for 6 days (day 6) and then exposed to adipogenic induction mixture in DMEM/H medium containing isobutylmethylxanthine (0.5 mM), dexamethasone (0.1 μM), human insulin (0.5 μM; Roche Applied Science, Indianapolis, Ind.), T3 (2 nM), indomethacin (30 μM), pantothenate (17 μM), biotin (33 μM) and 2% FBS for another 12 days (referred as day 18). Induction medium was changed every 3 days until collected.

### Generation of Immortalized Human Brown and White Fat Progenitors

Primary SVF cells were immortalized with human telomere reverse transcriptase (hTERT) as described previously (Tchkonia, T., et al. Diabetes 55, 2571-2578, 2006). Primary SVF that had undergone 4-5 population doublings were infected with a retrovirus containing the plasmid, pBABE-hTERT-Hygro (Addgene #1773, Cambridge, Mass.) that expresses hTERT driven by long terminal repeat promoter. Phoenix-A cells (ATCC) were infected with hTERT-Hygro DNA using PolyJet DNA in vitro transfection reagent (SignaGen Laboratories, Rockville, Md.). Culture supernatants containing virus were collected every 24 h after infection and filtered through a 0.45 um filter (Fisher Scientific, Pittsburgh, Pa.). Primary SVF cells from human white and brown fat at 80% confluence were infected with supernatants in the presence of 4 μg/mL Polybrene every day until cells reached 90% confluence. Cells were then treated with (concentrations ranging from 100 μg/mL to 400 μg/mL depending on cell conditions) in DMEM/H medium containing 10% FBS and antibiotics. Once drug selection was finished, the cells were maintained in culture medium with 50 μg/mL hygromycin for 2 weeks.

### Example 1: FGF6 Induces Uncoupling Protein 1 (UCP1) Expression in Brown Preadipocytes in the Absence of Differentiation, without Causing Cell Proliferation

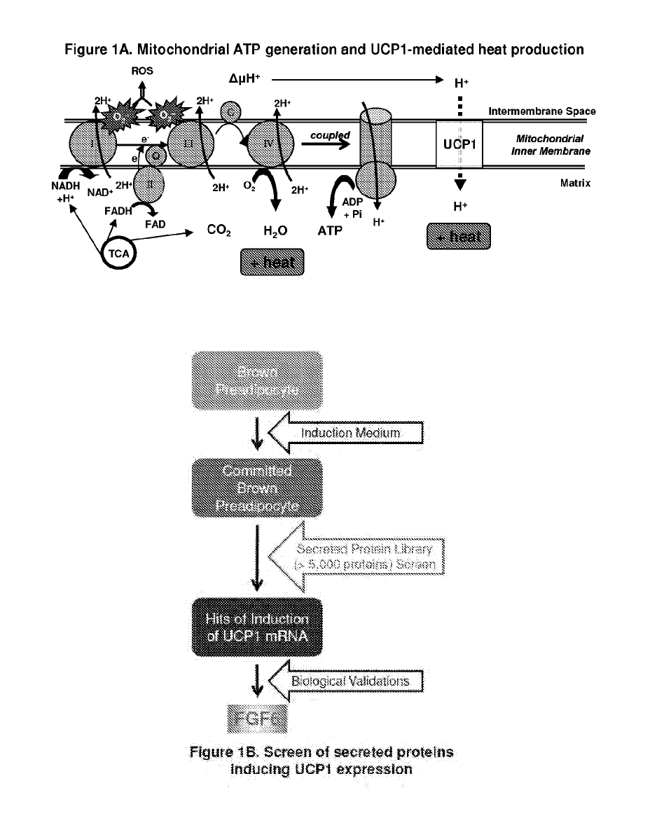

Brown adipocytes are characterized by multiple small lipid droplets and abundant mitochondria that oxidize nutrients and generate heat. Central to their thermogenic activity is UCP1, which is uniquely expressed in brown adipose tissue (BAT), and therefore serves as a defining marker of brown adipocytes. UCP1 is a 32 kDa inner mitochondrial transmembrane protein expressed only in brown adipocytes that allows protons in the mitochondrial intermembrane space to re-enter the mitochondrial matrix without generating ATP, i.e., uncoupling. Heat is generated directly by protons rushing down their electrochemical gradient and also indirectly by the subsequent increase in flux through the electron transport chain that follows (FIG. 1A). This process is also known as thermogenesis. UCP1 is unique to BAT and is necessary to mediate BAT thermogenesis. While other tissues possess different members of the UCP family, UCP1 is the only carrier that can promote heat production. Thus, UCP1-deficient mice are cold sensitive and exhibit increased susceptibility to diet-induced obesity. Conversely, transgenic mice with UCP1 expression in white fat display a lean phenotype.

To identify protein(s) that can induce UCP1 expression in brown preadipocytes, a high-throughput screen was performed using a protein library containing more than 5,000 mammalian secreted proteins (Lin H. et al., Science 320:807-811, 2008) in an immortalized murine brown preadipocyte cell line (Klein J. et al., J. Biol. Chem. 274:34795-34802, 1999; Tseng Y. H. et al., Nat. Cell. Biol. 7:601-611, 2005). The screen included applying induction media to brown preadipocyte cells, resulting in committed brown preadipocytes. The committed brown preadipocytes were then contacted with secreted proteins from the library, such that secreted proteins that induced UCP1 mRNA expression were identified for further analysis (FIG. 1B). The screen identified a number of molecules that induced UCP1 mRNA expression in the committed brown preadipocytes, including FGF6.

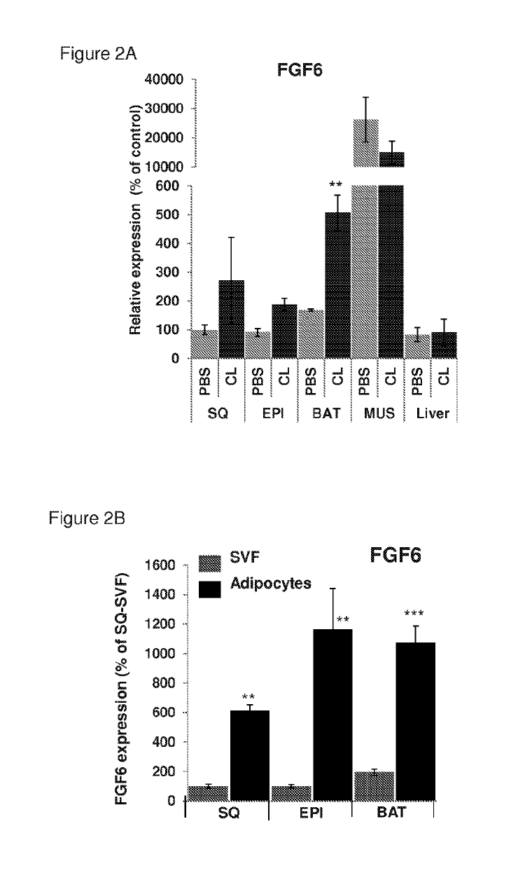

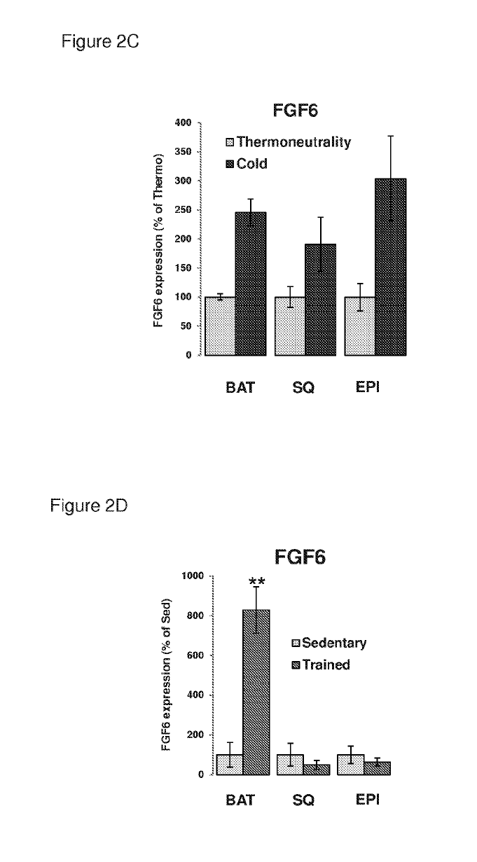

Analysis of FGF6 expression in different adipose tissues of C57BL/6 mice revealed that FGF6 was expressed in both brown adipose tissue (BAT) and white adipose tissue (WAT). Analysis in mice also showed that FGF6 mRNA expression (as measured by quantitative RT-PCR) levels were increased in both interscapular BAT and subcutaneous WAT (SQ) by β3-adrenergic agonist (CL316,243; referred to as “CL” in FIG. 2A) treatment relative to a PBS control, as described in FIG. 2A. CL316,243 was administered by intraperitoneal injection to the mice at a dose of 1 mg/kg body weight for 10 days.

FGF6 expression in adipocytes compared to cells within the stromal vascular fraction (SVF) was also examined to determine which cell type produces FGF6 within fat tissue. Cells within the SVF are progenitor cells, including preadipocytes. An analysis of FGF6 mRNA expression in adipocytes and SVF indicated that FGF6 was expressed in mature adipocytes at a much higher rate in all cell types tested (e.g., subcutaneous white adipose tissue (SQ), epididymal white adipose tissue (EPI), and brown adipose tissue (BAT)) as compared to the SVF fraction cells (including preadipocytes), as demonstrated in FIG. 2B.

The effect of temperature and exercise training on FGF6 expression levels were also analyzed. FGF6 expression was analyzed in brown adipose tissue (BAT), subcutaneous white adipose tissue (SQ), and epididymal white adipose tissue (EPI) following cold exposure or exercise training.

C57BL/6 mice were subjected to cold exposure by maintenance at 4° C. for 7 days. FGF6 mRNA levels were enhanced by cold exposure in BAT, SQ, and EPI (FIG. 2C). (See Schulz et al. (2013) Nature 495(7441): 379-83 for methods regarding cold exposure)

In addition, C57BL/6 mice were subjected to exercise training for 14 days. Two weeks of exercise training resulted in an 8-fold increase of FGF6 mRNA level in brown adipose tissue (BAT) (FIG. 2D) relative to SQ or EPI tissue. (See Stanford et al. (2015) Diabetes, epub agead of print PMID: 25605808).

The data provided in FIGS. 2A-2D suggests that FGF6 may serve as a local factor in response to sympathetic input, or to physiological stresses such as cold or exercise, to regulate UCP1-mediated thermogenesis.

### WT-1 Cell Differentiation Experiments

The effect of FGF6 on differentiation of WT-1 murine brown preadipocytes was also examined. WT-1 cells can be induced to undergo brown adipocyte differentiation, characterized by lipid accumulation and expression of adipogenic and brown fat markers, as described above (Klein et al. (1999) J Biol Chem 274:34795; Tseng et al. (2005) Nat Cell Biol 7:601). WT-1 cells were treated with FGF6 in growth media (DMEM+10% FBS), a vehicle control (“control” or “C” referred to in FIGS. 3A-D), or induction media (“induction” or “I” referred to in FIGS. 3A-D) as described above for seven days. Following the treatment, cells were examined for the color of the culture media in combination with oil Red O staining, expression of adipogenic markers, expression of brown fat markers, immunofluorescent staining of cells with UCP1 and DPAI, and Western blot analysis to determine the presence of UCP1 and β-tubulin. The results are described in FIGS. 3A to 3D.

mRNA expression of known adipogenic markers (PPARγ, aP2, and FAS) was measured in the WT-1 cells exposed to either the control, induction media, or FGF6. The results from this analysis are provided in FIG. 3A. The results show that only the induction media significantly increased each of the adipogenic markers relative to either FGF6 or the control.

mRNA expression of known brown fat markers in WT-1 cells was measured, where mRNA expression of PRDM16, PGC1α, CIDEA and UCP1 was examined based on exposure to the control, induction media, or FGF6. As described in FIG. 3B, FGF6 significantly increased mRNA expression of UCP1 in WT-1 cells as compared to either the control or the induction media. FGF6 did not, in contrast, have as significant an impact on the other brown fat markers, including PRDM16, PGC1α, and CIDEA.

Immunofluorescent staining of cells with UCP1 and DAPI showed that FGF6 treated cells displayed significant levels of staining for UCP1 protein relative to control cells. Further, as described in FIG. 3C, Western blot analysis revealed that UCP1 protein was also present in WT-1 cells exposed to FGF6, indicating that FGF6 induced both mRNA and protein expression of UCP1.

Surprisingly, in the absence of the induction cocktail, FGF6-treated cells displayed very little to no lipid accumulation by Oil Red O staining while expressing extremely high levels of UCP1 mRNA and protein (FIG. 3D).

The WT-1 cell experiments showed that FGF6 can induced UCP1 expression in the absence of lipid accumulation and expression of adipogenic markers (PPARγ, aP2, and FAS), and had the most significant impact on UCP1 expression of the brown fat markers tested. Accordingly, FGF6 induces UCP1 expression without causing differentiation of brown adipocytes.

### FGF6 Dose Response and Time Course Experiments

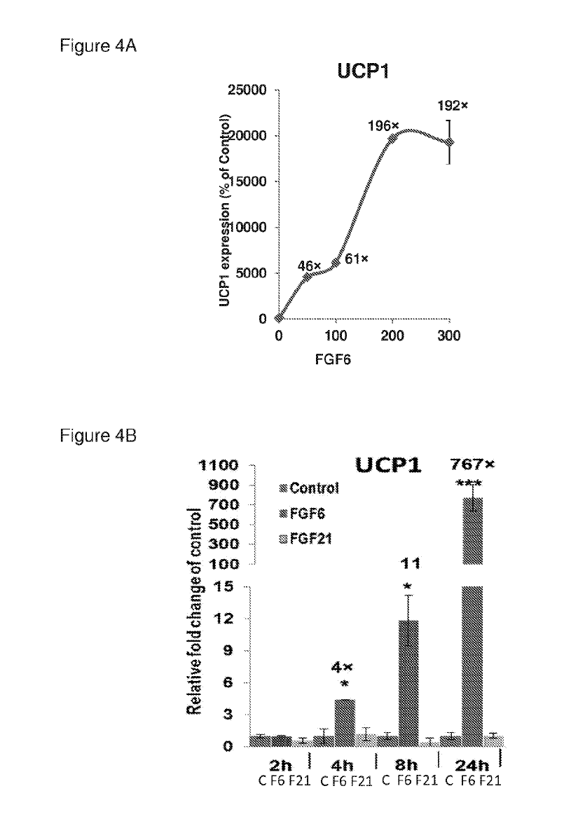

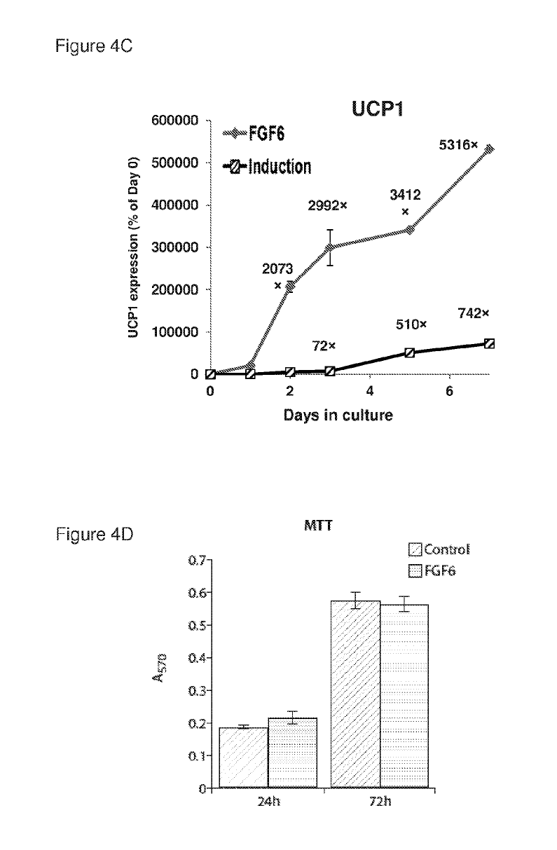

To better understand FGF6 regulation of UCP1 gene expression, dose-response and time-course experiments were performed. The results of these experiments are described in FIGS. 4A to 4C.

UCP1 mRNA expression in brown adipocyte cells was determined at FGF6 concentrations ranging from 0 to 300 ng/ml in the absence of induction cocktail. The results, shown in FIG. 4A, indicated that UCP1 gene expression could be increased by FGF6 in a dose-dependent manner, and plateaued at about 200 ng/ml of FGF6.

In order to determine the length of time necessary to induce UCP1 expression upon exposure to of cells to FGF6, an experiment was performed whereby FGF6 was administered to brown preadipocytes at a concentration of 200 ng/ml and UCP1 expression was determined. FGF21 and a control were also used in the time course experiment. FGF21, a member of the endocrine FGF ligands that has been implicated in the browning of white fat (Fisher F. M. et al., Genes Dev. 26:271-281, 2012). The results, shown in FIG. 4B, indicated that FGF6 regulated UCP1 expression within hours and in a time-dependent manner. FGF6 could acutely induce a significant increase of UCP1 expression as early as 4 hours after a 200 ng/ml dose, as described in FIG. 4B. At 24 hours, the fold-induction of UCP1 mRNA by FGF6 reached 700-fold higher than the untreated cells, as described in FIG. 4B.

To further extend the time response study, levels of UCP1 mRNA expression were examined following prolonged exposure of brown preadipocytes to FGF6. As described in FIG. 4C, UCP1 levels continued to rise with prolonged exposure to FGF6. FGF21 also had a marginal effect on UCP1 expression in the brown preadipocytes cultured in growth media (data not shown).

The effect of FGF6 on cell proliferation was also evaluated. As shown in FIG. 4D, when used at a concentration of 200 ng/ml, the optimal dosage for UCP1 induction in WT-1 cells, FGF6 had virtually no effect on cell proliferation in the WT-1 brown preadipocytes, eliminating the possibility that the induction of UCP1 by FGF6 was confounded by cell proliferation.

### Summary

In summary, the level of UCP1 mRNA expression induced by FGF6 greatly exceeded that of UCP1 mRNA induced by regular induction cocktail during the course of differentiation (compare FIG. 3B and FIG. 4B). Thus, FGF6 induced UCP1 expression without promoting differentiation of brown adipocytes. Together, these data reveal a previously unknown phenomenon: UCP expression induced by a fibroblast growth factor (FGF) in preadipocytes is dissociated from lipid accumulation and differentiation.

### Example 2: Overexpression of FGF6 in Brown or White Preadipocyte Cell Lines Induces UCP1 Expression and Mitochondrial Respiration

In FGF6-treated cells, a surprisingly high level of UCP1 expression was observed (see FIGS. 3A-D and 4A-C), as well as a surge in acidification of culture media indicating increased mitochondrial metabolism (FIG. 3D). To assess FGF6's role in the regulation of mitochondrial activity, FGF6 was stably expressed in WT-1 brown preadipocytes and 3T3-F442A white preadipocytes. Stable cells were generated by viral infection followed by drug selection.

### Mitochondrial Respiration and Activity

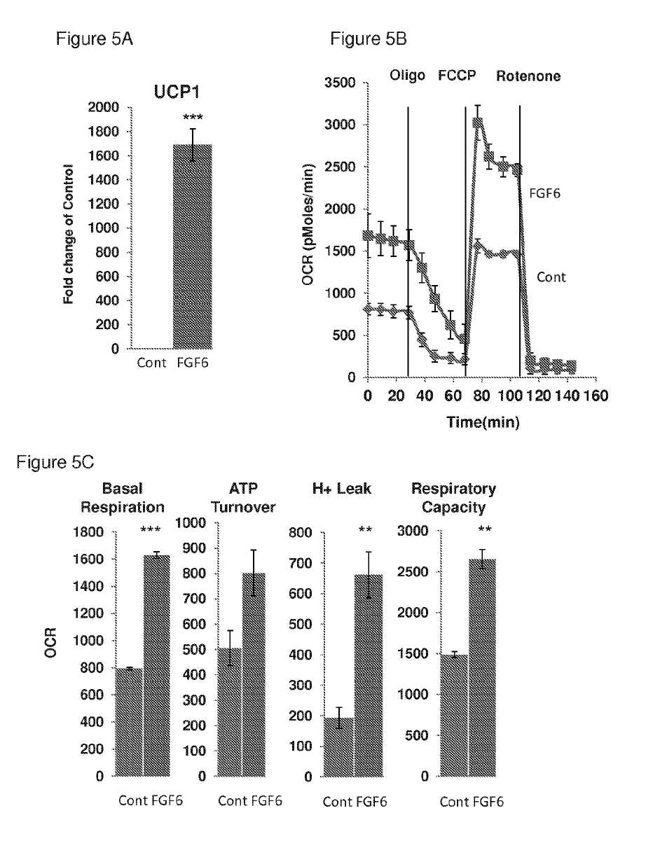

Consistent with the findings described above, overexpression of FGF6 greatly increased UCP1 expression over basal level in WT-1 brown preadipocytes, as described in FIG. 5A. When placed on the Seahorse Bioanalyzer for analysis of bioenergetic potential, these cells displayed robust increases in mitochondrial activity when abundant nutrients were provided (10 mM glucose, 0.5 mM carnitine, and 1 mM palmitate-BSA). A profile of cellular respiration was developed by utilizing well-characterized mitochondrial toxins, as described in FIG. 5B. First, basal respiration was measured, followed by injection of oligomycin (an inhibitor of ATP synthase which allows measurement of ATP turnover). Then, the uncoupler FCCP (carbonilcyanide p-triflouromethoxyphenylhydrazone) was injected to measure respiratory capacity, followed by the complex 1 inhibitor rotenone (which prevents electron transfer activity and leaves only non-mitochondrial activity to be measured). The bioenergetic profile of FGF6 overexpressing brown preadipocytes versus control cells not exposed to FGF6 revealed an increase in all tested aspects of cellular respiration, including basal respiration, ATP turnover, proton leak, and respiratory capacity, as described in FIG. 5C.

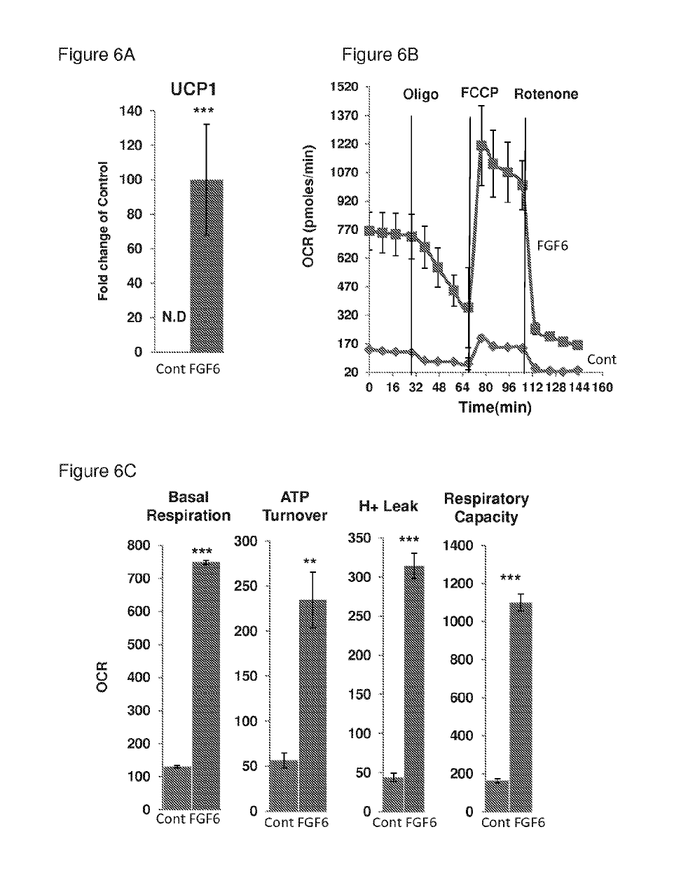

As mentioned above, 3T3-F442A white preadipocytes were also used to determine FGF6's role in mitochondrial activity regulation. Constitutive overexpression of FGF6 in 3T3-F442A white preadipocytes resulted in UCP1 expression, which is unusual for white preadipocytes, as described in FIG. 6A. These white preadipocytes also displayed highly-elevated mitochondrial activity, with 5-10 fold increases in cellular respiration, including an approximate 7-fold increase of basal respiration, a 5-fold gain of ATP turnover and proton leak, and a nearly 6-fold increase of maximal respiratory capacity, as described in FIGS. 6B-6C. These data demonstrate the FGF6 induced UCP1 expression is coupled to an increase in cellular energy consumption, as the energy consumption was observed in both brown and white preadipocytes.

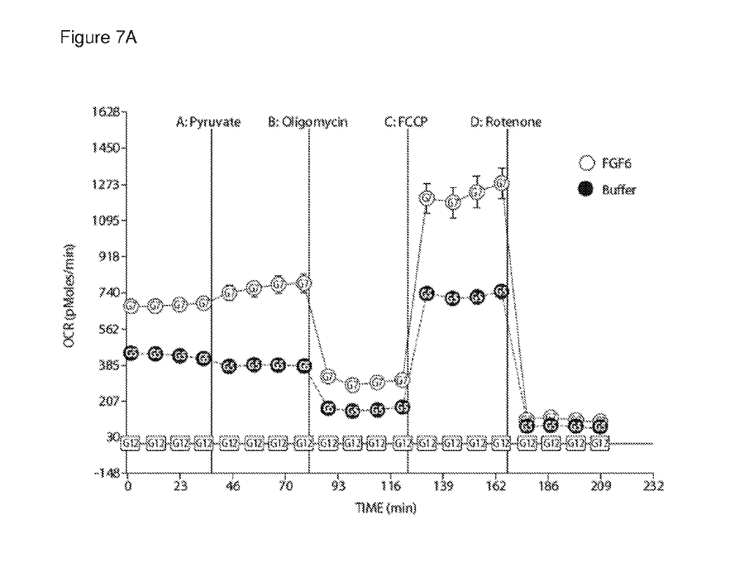

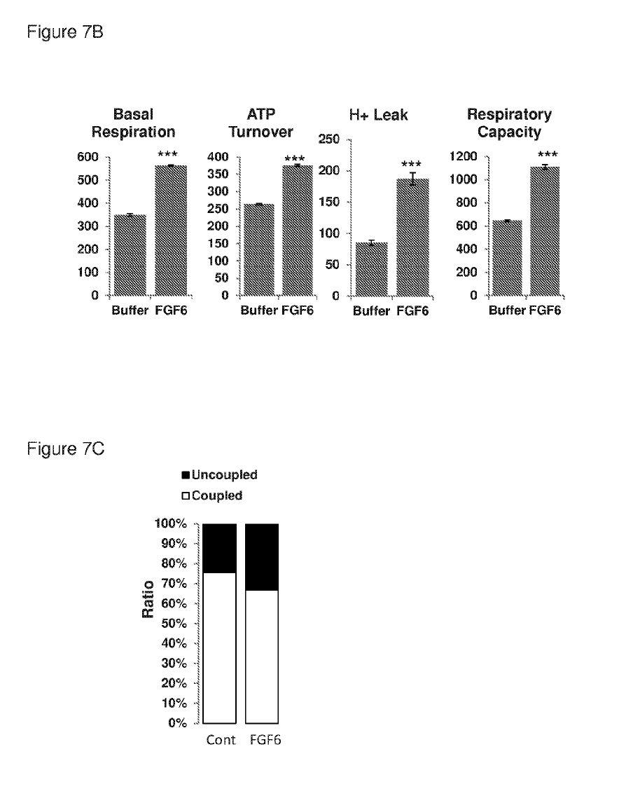

Similar results were observed when WT-1 brown preadipocytes were treated with 200 ng/mL of FGF6 for 24 hrs using the Seahorse Bioanalyzer. When placed on the Seahorse Bioanalyzer for analysis of bioenergetics potential, these cells displayed robust increases in mitochondrial activity when abundant nutrients were provided (10 mM glucose, 0.5 mM carnitine, and 1 mM palmitate-BSA). The bioenergetic profile of FGF6 treated brown preadipocytes versus control cells revealed a coordinated increase in all aspects of cellular respiration, including basal respiration, ATP turnover, proton leak, and respiratory capacity (FIG. 7A-B). The increased level of proton leak indicates an elevation in uncoupled respiration, as indicated in FIG. 7C, and suggests that the UCP1 protein induced by FGF6 in the preadipocytes is actively uncoupling respiration from ATP synthesis and facilitating fuel utilization.

### Mitochondrial Dynamics and Biogenesis

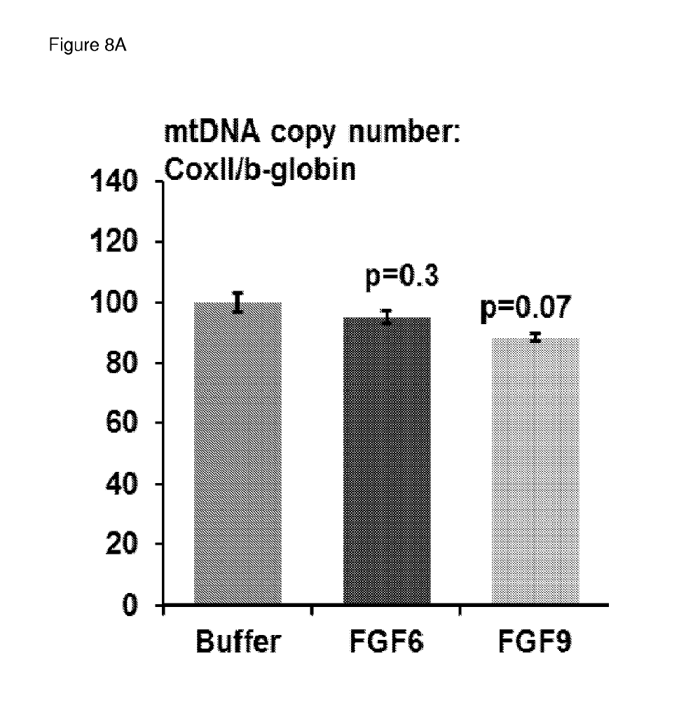

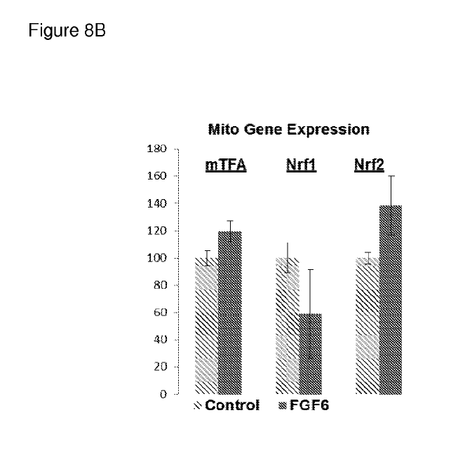

In order to determine whether the marked changes in mitochondrial respiration observed in preadipocytes treated with FGF6 is due to increased mitochondrial mass and/or changes in mitochondrial dynamics, in addition to increased UCP1 expression, mitochondrial DNA (mtDNA) copy number (as the ratio of CoxII (mtDNA) over β-globin (nuclear DNA)) and expression of genes involved in mitochondrial replication (e.g., mTFA, Nrf1 and Nrf2) in brown preadipocytes was measured. As indicated in FIG. 8A, no significant difference in mitochondrial DNA copy number was observed between control and FGF6-treated cells. Similarly, expression of nuclear-encoded mitochondrial genes was not significantly altered upon treatment with FGF6, as shown in FIG. 8B. These data suggest that FGF6 regulates mitochondrial activity without changing mitochondrial copy number.

To determine if FGF6 treatment affects the overall health condition of mitochondria, mitochondrial attributes such as mitochondria biogenesis, mass, morphology and dynamics were measured. Mitochondrial mass was measured by a cell-permeable dye (MitoTracker Green FM) and by transmission electron microscopy (EM). MitoTracker Green FM accumulates in mitochondria in live cells irrespective of mitochondrial membrane potential. The intensity of fluorescence was visualized by microscopy and quantitated by flow cytometry. Mitochondrial morphology and ultra-structure (such as cristae and granules) was assessed by EM. Using transmission electron microscopy to examine FGF6 expression, it was determined there were fewer mitochondria in preadipocytes that overexpressed FGF6, and these mitochondria had a longer shape as compared to the control.

### Example 3: FGF6 Induces UCP1 Expression in Primary Adipose Progenitors, but has No Apparent Effect on Myogenic Progenitors

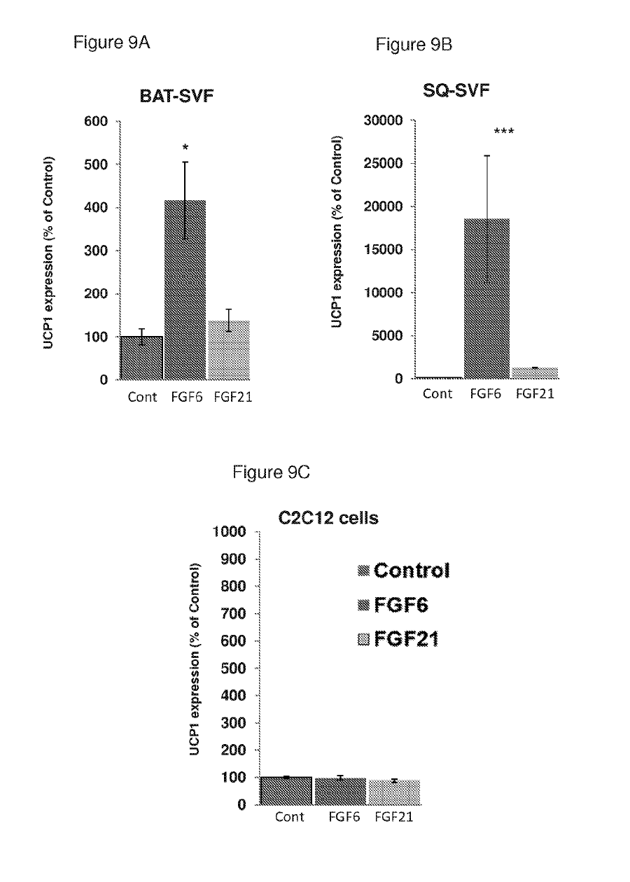

To determine if FGF6 treatment of primary adipose progenitors could also increase UCP1 expression as observed in Example 1, stromo-vascular fraction (SVF) cells, which comprise adipocyte progenitors, were isolated from interscapular brown adipose tissue (BAT-SVF) and subcutaneous white adipose tissue (SQ-SVF). SVF cells were subsequently treated with FGF6 or FGF21 in growth media (DMEM+10% FBS) for 3 days, followed by determination of UCP1 expression. The results of the experiments are provided in FIGS. 9A-C. As described in FIGS. 9A and 9B, FGF21 treatment had little effect on UCP1 mRNA expression in either type of SVF cells, as compared to the control. In contrast, FGF6 induced a 4-fold increase of UCP1 mRNA expression in BAT-SVF (FIG. 9A), and a nearly 200-fold increase in UCP1 mRNA expression in SVF derived from subcutaneous WAT (FIG. 9B) as compared to the control.

These data indicate that FGF6 functions as a “browning” factor by induction of UCP1 in progenitors derived from white fat as well as from brown fat. In contrast, neither FGF6 nor FGF21 induced UCP1 mRNA expression in the C2C12 myogenic progenitors, as described in FIG. 9C. These results indicated that the effect of FGF6 on UCP1 induction is specific to adipose progenitor cells.

### Example 4: FGF6 Increases the Expression of COX2, an Inducer of Browning, and Suppresses the Expression of RIP140, an Inhibitor of Brown Adipocyte Differentiation

To determine the molecular mechanism(s) by which FGF6 regulates UCP1 expression and mitochondrial activity, the expression of transcriptional regulators of UCP1 were studied by treating WT-1 brown adipocytes with 50 ng/mL, 100 ng/mL, 200 ng/mL or 300 ng/mL of FGF6 in growth media (DMEM+10% FBS) for three days.

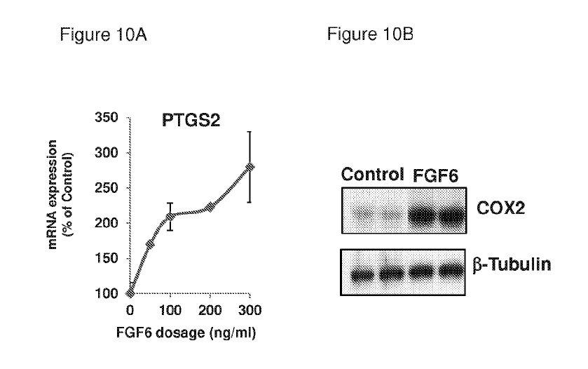

FGF6 did not increase the expression of known transcriptional regulators of UCP1, such as PGC1α, PPARγ, and PRDM16. However, FGF6 induced the expression of PTGS2 mRNA (FIG. 10A). FGF6 also increased the expression of cyclooxygenase-2 (COX2) protein, as demonstrated by Western blot analysis, in a dose-dependent manner in the WT-1 brown preadipocytes (FIG. 10B).

PTGS2 is the gene encoding COX2, a rate-limiting enzyme in prostaglandin (PG) synthesis, and this pathway has been linked to brown fat recruitment (Madsen L. et al., PLOS One 5:e11391, 2010; Vegiopoulos A. et al., Science 328:1158-1161, 2010). These data indicate that the COX2-PG pathway mediates, at least in part, the effect of FGF6 on UCP1 mRNA induction.

To determine if the COX-2 prostaglandin pathway is required for FGF6 induction of UCP1 expression, WT-1 brown preadipocytes were pretreated with the COX2 selective inhibitor NS-398 (Shen W. et al., Am. J. Pathol. 167:1105-1117, 2005) at concentrations of 0 uM, 10 uM, 20 uM or 50 uM. Following NS-398 treatment, the cells were treated with 200 ng/mL FGF6 in growth media (DMEM+10% FBS) for three days.

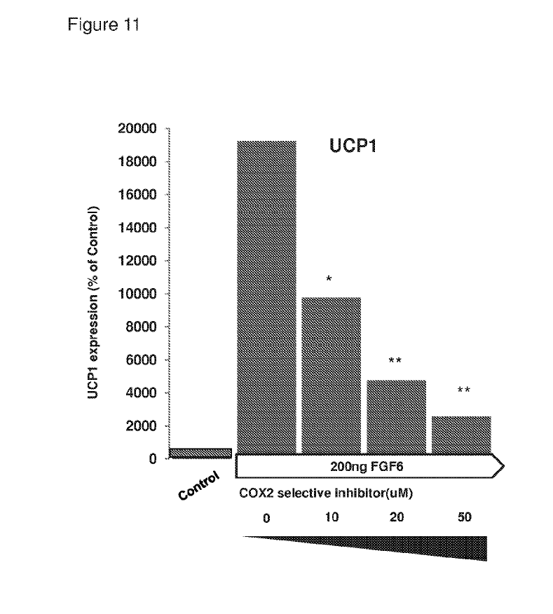

UCP1 mRNA expression was determined and demonstrated that the COX2 inhibitor inhibited UCP1 expression in a dose dependent manner (FIG. 11).

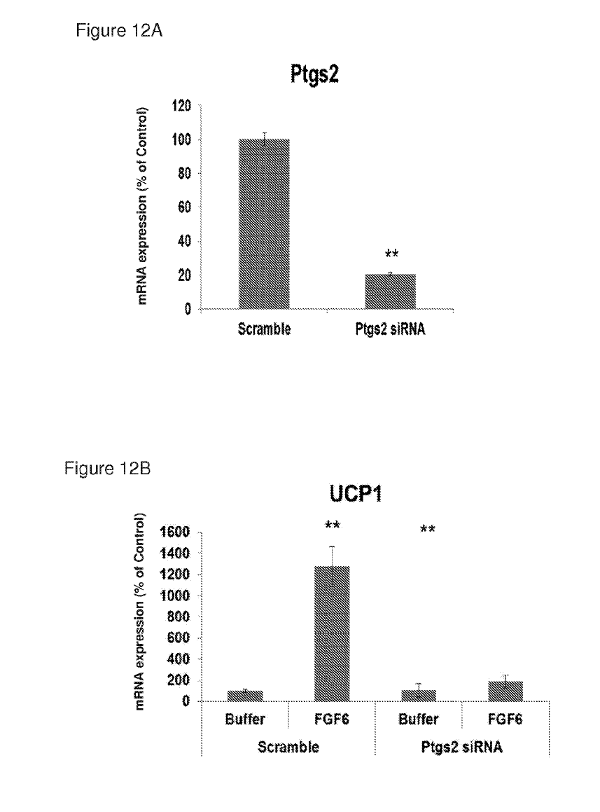

The results were further confirmed by siRNA experiments. Mouse brown preadipocytes, DE cells (Pan et al, (2009), Cell, 137: 73-86) were infected with lentivirus expressing PTGS2 siRNA or a control scramble siRNA (non specific siRNA). After drug selection, the stable cells were treated with 200 ng/ml of FGF6 for 48 hours. Expression of PTGS2 was evaluated by QPCR. FIGS. 12A and 12B show that stable transfection of PTGS2 specific siRNA to cells resulted in a loss of PTGS2 expression, and abolished the effect of FGF6 on UCP1 mRNA induction. As shown above, these data also indicate that the COX2-PG pathway mediates, at least in part, the effect of FGF6 on UCP1 mRNA induction.

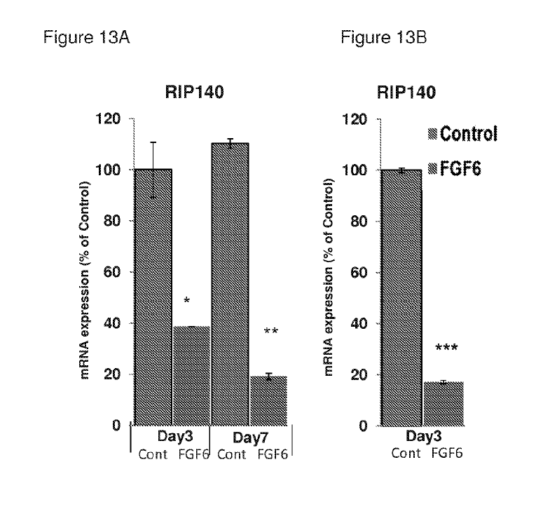

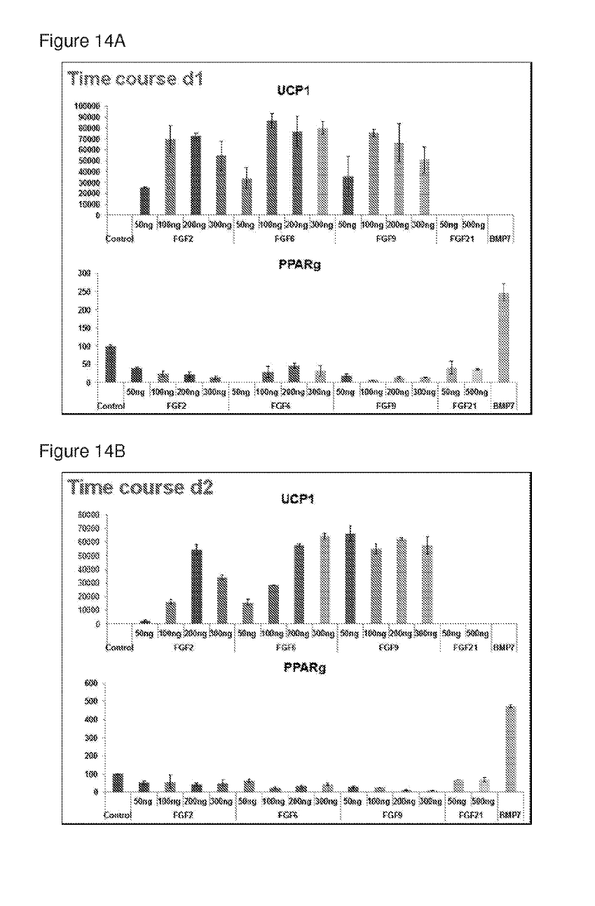

Nuclear receptor interacting protein 1 (NRIP1), also known as receptor interacting protein 140 (RIP140), was also studied to determine its role in FGF6-induced UCP1 mRNA expression. RIP140 directs DNA methylation and interacts with nuclear receptors to silence UCP1 expression and suppress mitochondrial biogenesis in white adipocytes (Kiskinis E. et al., EMBO J. 26:4831-4840, 2007; Powelka A. M. et al., J. Clin. Invest. 116:125-136, 2006; Wang H. et al., Mol. Cell. Biol. 28:2187-2200, 2008). RIP140 knockout mice are lean with increased energy expenditure and are resistant to high-fat diet-induced obesity. WAT of RIP140 null mice displays genes characteristic of BAT, including UCP1 and CIDEA. In addition, RIP140 interacts with liver X receptor α (LXRα) to suppress UCP1 gene expression and brown fat phenotype. Treatment of brown and white preadipocytes with 200 ng/mL FGF6 in growth media (DMEM+10% FBS) for either three or seven days resulted in a 50-80% reduction of RIP140 mRNA expression (FIGS. 13A (brown preadipocytes) and 13B (white preadipocytes)) indicating that FGF6 suppresses the expression of RIP140, an inhibitor of brown adipocyte differentiation.

These results indicate that the induction of UCP1 by FGF6 is due, at least in part, to its ability to induce an activator (e.g., COX2-PG) and suppress a repressor (e.g., RIP140) of UCP1 transcription. These pathways appear to target UCP1 expression and regulate mitochondrial function without causing lipid accumulation in precursor cells that are committed to an adipocyte fate.

### Example 5: FGF2, FGF6 and FGF9 Induce Expression of UCP1 mRNA in Murine Brown Preadipocytes in a Dose Responsive Manner

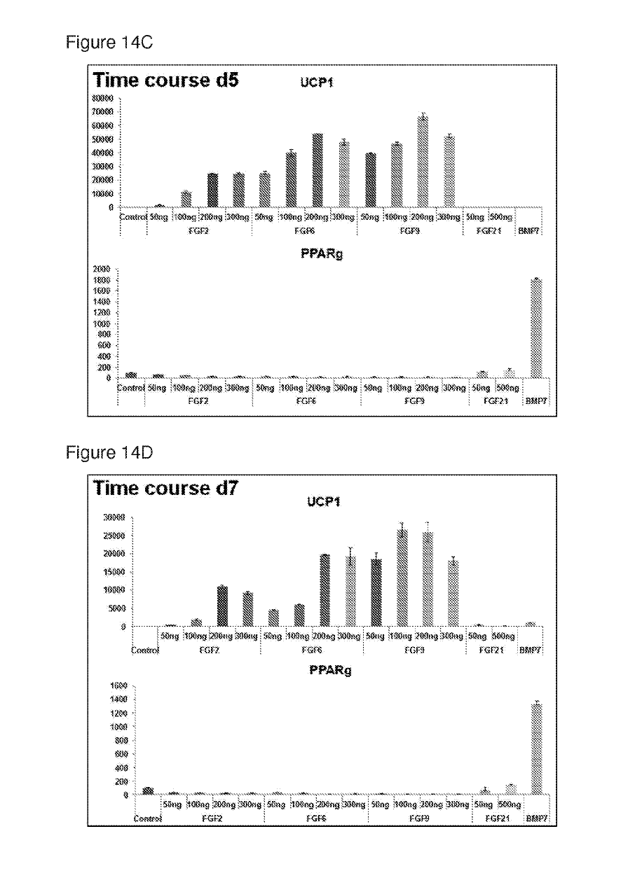

To determine if other FGF proteins were able to induce UCP1 mRNA expression in a dose dependent manner, murine brown preadipocyte WT-1 cells (Tseng et al., Nat. Cell. Biol. (2005) 7(6):601-611) were grown in growth media (DMEM-high glucose+10% FBS) supplemented with FGF2, FGF6, FGF9, FGF21, BMP7 or vehicle (control) in combination with insulin (20 nM) and triidothyronine (T3, 1 nM). FGF2, FGF6 and FGF9 were used at a dosage of 50 ng/mL, 100 ng/mL, 200 ng/mL or 300 ng/mL. FGF21 was used at the dosage of 50 ng/ml and 500 ng/mL. The concentration of BMP7 was 3.3 nM. RNA was isolated and UCP1 and PPARγ expression levels were evaluated by quantitative reverse transcription polymerase chain reaction (Q-RT-PCR) analysis after 24 hours (i.e., 1 day) (FIG. 14A), 2 days (FIG. 14B), 5 days (FIG. 14C) and 7 days (FIG. 14D) of treatment. All experiments were performed triplicate and the data are presented as mean+/−SEM.

At each time point and at each dose, the FGF2, FGF6 and FGF9 treated cells expressed very low levels of PPARγ and very high levels of UCP1 in a dose-responsive manner. FGF21 or BMP7 treated cells did not express detectable amounts of UCP1 at days 1, 2, and 5, and only minimal amounts of UCP1 was detected at day 7. These data demonstrate that, in addition to FGF6, FGF2 and FGF9 can also induce UCP1 expression and that the induction is in the absence of induction of adipogenic markers (i.e., PPARγ).

### Example 6: Time Course Experiments Studying FGF2, FGF6 and FGF9 Induced Expression of UCP1 mRNA in Murine Brown Preadipocytes

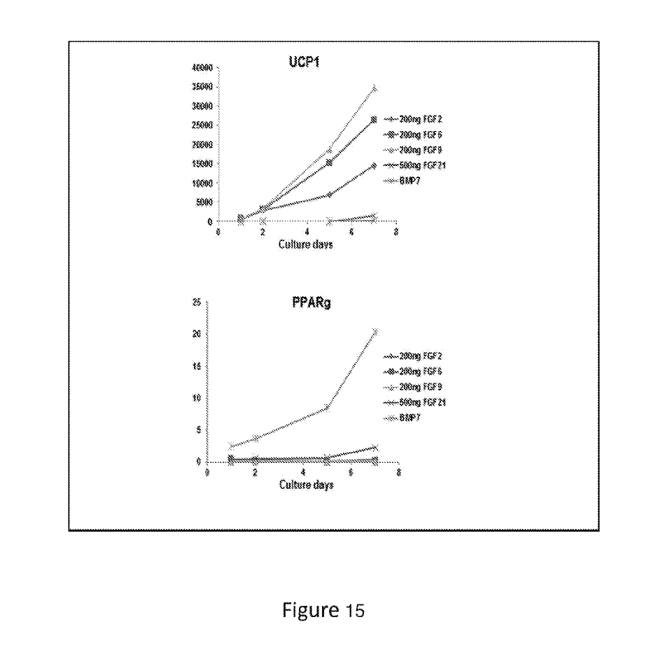

To determine if FGF2, FGF6 and/or FGF9 were able to induce UCP1 mRNA expression over time, murine brown preadipocyte WT-1 cells (Tseng et al., Nat. Cell. Biol. (2005) 7(6):601-611) were grown in growth media (DMEM-high glucose+10% FBS) supplemented with FGF2, FGF6, FGF9, FGF21 or BMP7 in combination with insulin (20 nM) and triidothyronine (T3, 1 nM). FGF2, FGF6 and FGF9 were used at a dosage of 200 ng/mL. FGF21 was used at a dosage of 500 ng/mL. The concentration of BMP7 was 3.3 nM. mRNA was isolated and UCP1 and PPARγ expression levels were evaluated by quantitative reverse transcription polymerase chain reaction (Q-RT-PCR) analysis after 24 hours (i.e., 1 day), 2 days, 5 days and 7 days of treatment (FIG. 15). All experiments were performed triplicate and the data are presented as mean+/−SEM.

At each time point, the FGF2, FGF6 and FGF9 treated cells expressed very low levels of PPARγ and very high levels of UCP1 in a dose-responsive manner. FGF21 treated cells did not express detectable amounts of UCP1. These data also demonstrate that FGF2, FGF6 and FGF9 can induce UCP1 expression in the absence of induction of adipogenic markers (i.e., PPARγ).

### Example 7: FGF2, FGF6 and FGF9 Induce Expression of UCP1 Protein in Murine Brown Preadipocytes

To determine if FGF2, FGF6 and/or FGF9 were able to induce UCP1 protein expression, murine brown preadipocyte WT-1 cells (Tseng et al., Nat. Cell. Biol. (2005) 7(6):601-611) were grown in growth media (DMEM-high glucose+10% FBS) supplemented with FGF2, FGF6, FGF9 or vehicle (control) in combination with insulin (20 nM) and triidothyronine (T3, 1 nM). FGF2, FGF6 and FGF9 were used at a dosage of 200 ng/mL. Cells were stained using immunofluorescent stains for UCP1 and DAPI (which binds to DNA).

The immunofluorescent staining showed that FGF2, FGF6 and FGF9 treated cells displayed significant levels of staining for UCP1 protein relative to control cells, indicating high levels of UCP1 protein. These data demonstrate that FGF2, FGF6 and FGF9 also induced expression of UCP1 protein.

### Example 8: FGF4 Induces Expression of UCP1 mRNA in Murine Brown Preadipocytes

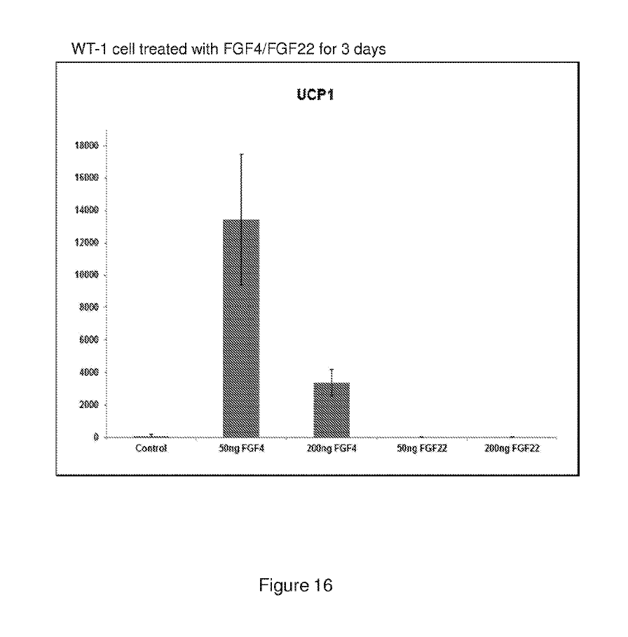

To determine if FGF4 and/or FGF22 were able to induce UCP1 mRNA expression, murine brown preadipocyte WT-1 cells (Tseng et al., Nat. Cell. Biol. (2005) 7(6):601-611) were grown in growth media (DMEM-high glucose+10% FBS) supplemented with FGF4, FGF22 or vehicle (control) in combination with insulin (20 nM) and triidothyronine (T3, 1 nM) for three days. FGF4 and FGF22 were used at a dosage of 50 ng/mL and 200 ng/mL. mRNA was isolated and UCP1 gene expression was evaluated by quantitative reverse transcription polymerase chain reaction (Q-RT-PCR) analysis (FIG. 16). All experiments were performed triplicate and the data are presented as mean+/−SEM.

The data demonstrated that FGF4 treated cells displayed high levels of UCP1 mRNA expression. In contrast, expression of UCP1 was not detectably induced in the FGF22 treated cells at the dosages tested.

### Example 9: FGF4, FGF6, FGF17, FGF18 and FGF20 Induce Expression of UCP1 and PTGS2 mRNA in Murine Brown Preadipocytes

To determine if FGF4, FGF5, FGF6, FGF10, FGF16, FGF17, FGF18 or FGF20 induced either UCP1 mRNA or PTGS2 mRNA expression, murine brown preadipocyte WT-1 cells (Tseng et al., Nat. Cell. Biol. (2005) 7(6):601-611) were grown in growth media (DMEM-high glucose+10% FBS) supplemented with FGF4, FGF5, FGF6, FGF10, FGF16, FGF17, FGF18, FGF20 or buffer (control) in combination with insulin (20 nM) and triidothyronine (T3, 1 nM) for three days. All FGFs were used at a dosage of 200 ng/mL. mRNA was isolated and UCP1 and PTGS2 gene expression levels were evaluated by quantitative reverse transcription polymerase chain reaction (Q-RT-PCR) analysis. All experiments were performed triplicate and the data are presented as mean+/−SEM.

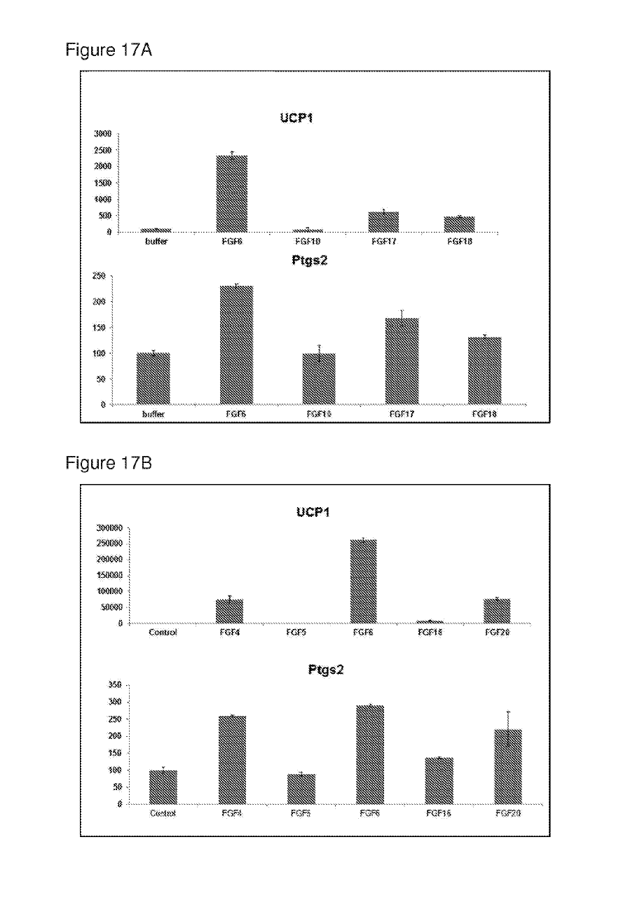

Treatment of the cells with FGF4, FGF6, FGF17, FGF18 and FGF20 induced a 5-fold or higher expression of UCP1 mRNA relative to the control (FIGS. 17A and 17B). In contrast, expression of UCP1 was not induced in the FGF5 and FGF10 treated cells at the dosage tested (FIGS. 17A and 17B). FGF16 induced minimal expression of UCP1 (FIG. 17B). PTGS2 mRNA expression was increased in the FGF4, FGF6, FGF17, FGF18 and FGF20 treated cells relative to the control, but not in the FGF5, FGF16 and FGF10 treated cells. These data demonstrate that FGF4, FGF6, FGF17, FGF18 and FGF20 also induced expression of UCP1 mRNA and PTGS2 mRNA, the gene encoding COX2. These data also indicate that the COX2-PG pathway mediates, at least in part, the effect of FGF4, FGF6, FGF17, FGF18 and FGF20 on UCP1 mRNA induction.

### Example 10: FGF1 Induces Expression of UCP1 and PTGS2 mRNA in Murine Brown Preadipocytes

To determine if FGF1 or FGF10 induce UCP1 mRNA expression or PTGS2 mRNA expression, murine brown preadipocyte WT-1 cells (Tseng et al., Nat. Cell. Biol. (2005) 7(6):601-611) were grown in growth media (DMEM-high glucose+10% FBS) supplemented with FGF1, FGF10 or vehicle (control) in combination with insulin (20 nM) and triidothyronine (T3, 1 nM) for three days. FGF1 and FGF10 were used at a dosage of 50 ng/mL, 100 ng/mL or 300 ng/mL. mRNA was isolated and UCP1 and PTGS2 expression levels were evaluated by quantitative reverse transcription polymerase chain reaction (Q-RT-PCR) analysis. All experiments were performed triplicate and the data are presented as mean+/−SEM.

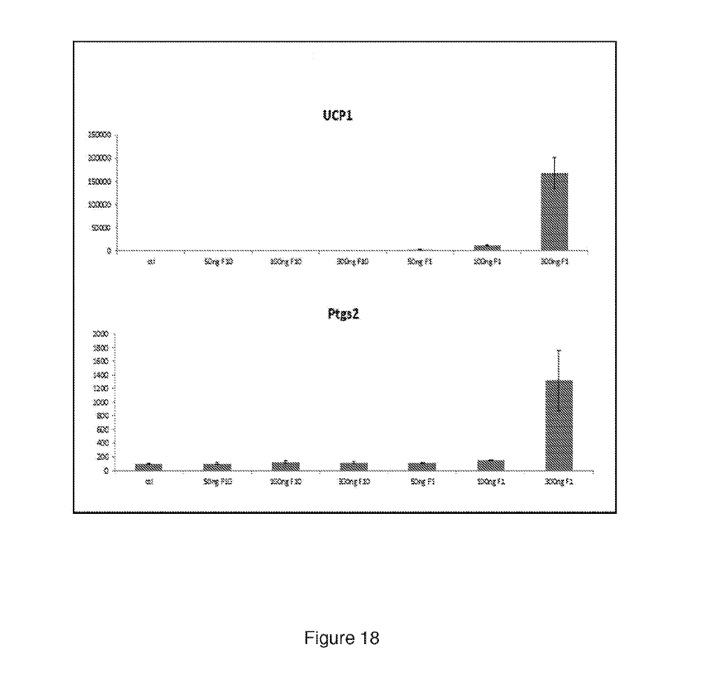

Treatment with FGF1 at a dose of 300 ng/ml induced expression of UCP1 and PTGS2. FGF10 did not induce expression of either UCP1 or PTGS2 mRNA (FIG. 18). These data indicate that the COX2-PG pathway also mediates, at least in part, the effect of FGF1 on UCP1 mRNA induction.

### Example 11: FGF Induces UCP1 Expression in Differentiated Cells

To determine whether FGF could induce UCP1 expression in differentiated cells, WT-1 murine brown preadipocytes that has been induced to differentiate were exposed to various FGFs and tested for UCP1 expression.

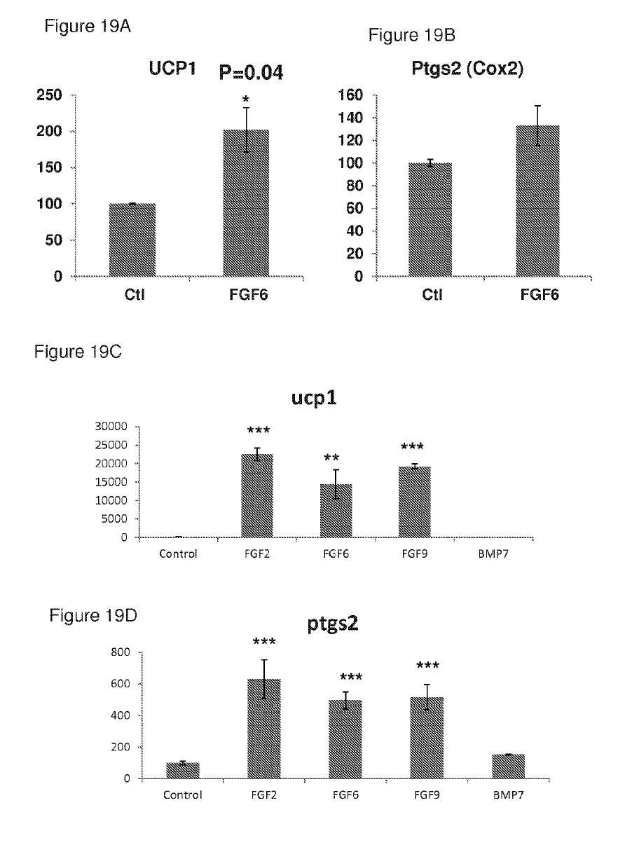

WT-1 preadipocyte cells were exposed to 3.3 nM BMP7 in growth media (DMEM-high glucose, 10% FBS, supplemented with 20 nM insulin and 1 nM triiodothyronine (T3)) for 8 days in order to induce differentiation of the adipocytes (Tseng Y. H. et al., (2008) Nature 454(7207):1000-1004). Following day 8 of treatment, the cells were exposed to 200 mg/mL of FGF6 protein, or vehicle control, in growth media for 32 hours. Cells were then collected, and UCP1 and PTGS2 mRNA expression levels were determined according to the quantitative RT-PCR assay described above. The results are described in FIG. 19A and indicate that FGF6 induced UCP1 expression in cells differentiated using BMP7 relative to cells treated with vehicle alone (i.e., control cells). As described in FIG. 19B, PTGS2 levels were also increased in the differentiated WT-1 cells exposed to FGF6.

In a separate experiment investigating the ability of FGF proteins to induce UCP1 expression in cells undergoing adipocyte differentiation, WT-1 preadipocyte cells were differentiated by culturing in growth media supplemented with insulin (20 nM) and triiodothyronine (T3, 1 nM) for 3 days, followed by incubation in an induction media (growth medium supplemented 20 nM insulin, 1 nM T3, 0.5 mM isobutyl-methylxanthine, 5 μM dexamethasone) for 2 days. The cell were then exposed to BMP7 (3.3 nM) or 200 ng/mL of FGF2, FGF6 or, FGF9 in growth media supplemented with insulin (20 nM) and triodothyronine (T3, 1 nM) for 2 additional days. Cells were harvested and UCP1 and PTGS2 mRNA expression levels were determined according to the quantitative RT-PCR assay described above. Experiments were performed in triplicate and the data presented as mean+/−SEM. The results are provided in FIGS. 19C and 19D and indicate that each of the FGFs tested were able to induce UCP1 and PTGS2 mRNA expression. In contrast, the cells exposed to BMP7 or the cells exposed to neither BMP7 nor an FGF protein (control), expressed minimal levels of UCP or PTGS2 mRNA.

Thus, as described in FIGS. 19A-19D, FGF proteins were able to induce UCP1 and PTGS2 expression in differentiated cells.

### Example 12: FGF6 Regulates Fuel Utilization in Preadipocytes

To determine whether nutrient supply could regulate mitochondrial activity in FGF6-treated cells, concentrations of glucose in the assay media of cells were manipulated and measured in the Seahorse Bioanalyzer. FGF6 was stably expressed in WT-1 brown preadipocytes and 3T3-F442A white preadipocytes. Stable cells were generated by viral infection followed by drug selection.

### Glycolysis and Glucose Uptake

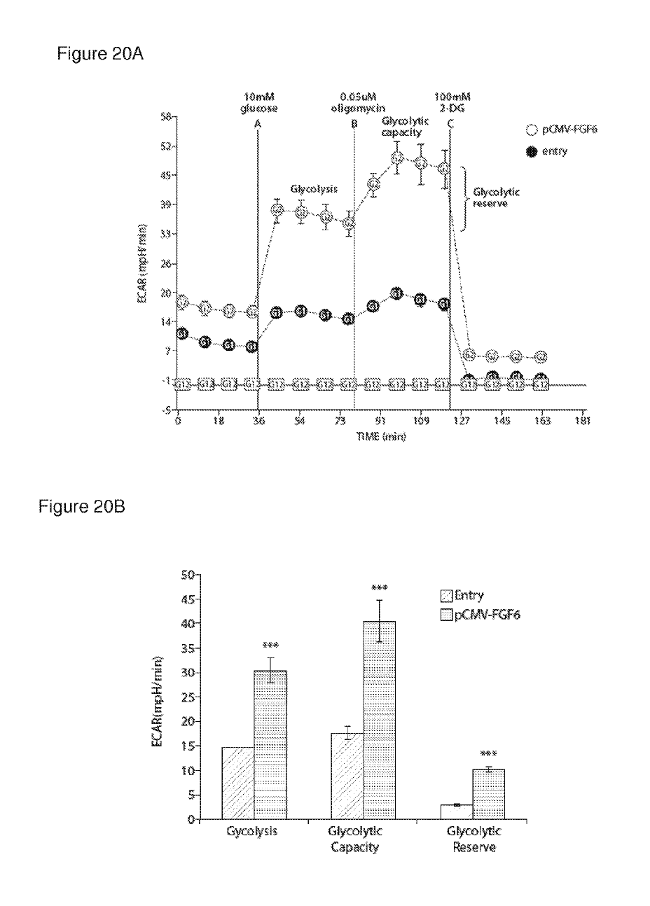

A profile of the extracellular acidification rate (ECAR) was developed as described in FIG. 20A. 10 mM glucose was added to the assay media, which was then taken up by the cells and catabolized through glycolysis, producing ATP and protons, and resulting in a rapid increase in ECAR. Subsequently, oligomycin was injected which inhibits mitochondrial ATP production and thus shifts the energy production to glycolysis, with the subsequent increase in ECAR revealing the maximum glycolytic capacity of the cells. Lastly, the glycolysis inhibitor 2-DG was added to measure glycolytic reserve. The bioenergetic profile of FGF6 overexpressing brown preadipocytes versus control cells not exposed to FGF6 revealed an increase in cellular glycolysis, glycolytic capacity and glycolytic reserve, as described in FIG. 20B, suggesting that the UCP1 protein induced by FGF6 in the preadipocytes is actively facilitating glucose utilization.

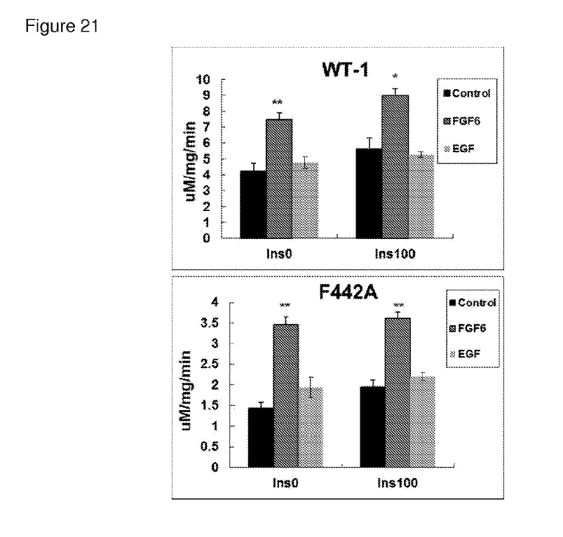

The following glucose uptake assay was used to determine whether FGF6 could impact cellular glucose uptake. Specifically, glucose uptake was monitored in WT-1 brown preadipocytes and 3T3-F442A white preadipocytes in the presence or absence of FGF6. After serum starvation in DMEM/H medium containing 1% of BSA for 2-3 h, cells were washed with HEPES buffer and then incubated with or without 100 nM insulin for 30 min in DMEM/H medium containing 1% of BSA. Glucose transport was determined by the addition of 2-deoxy-[3H]glucose (0.1 mM, 0.5 μCi/mL; PerkinElmer Life and Analytical Science, Waltham, Mass.). After 5 minutes of incubation, the reaction was stopped by ice-cold PBS and cells were washed twice with ice-cold PBS. Cells were then lysed in 0.1% SDS, and glucose uptake was assessed in 4 mL of scintillant using Beckman LS6500 scintillation counter (Beckman Coulter, Indianapolis Ind.). Nonspecific 2-deoxy-[3H]glucose uptake was measured in the presence of cytochalasin B (20 μM) and was subtracted from the total uptake to get specific glucose uptake. Results were expressed as the mean±s.e.m. of the indicated number of experiments. The protein content was determined by the Bradford method. As shown in FIG. 21, treatment with FGF6 increased glucose uptake level in both WT-1 (brown) and F442A (white) preadipocytes, whereas treatment with EGF and the control had no substantial effect.

### Example 13: FGF6 does not Induce UCP1 Expression in Mature Brown Adipocytes, but Increases Oxygen Consumption and Glucose Uptake Levels

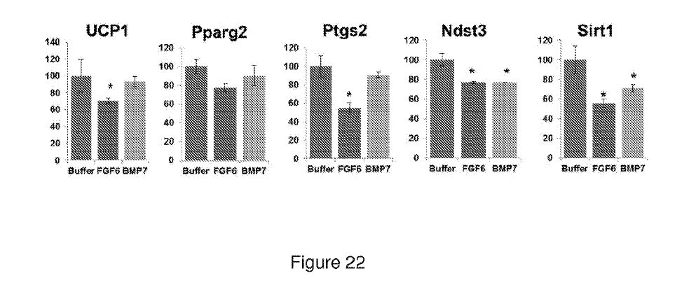

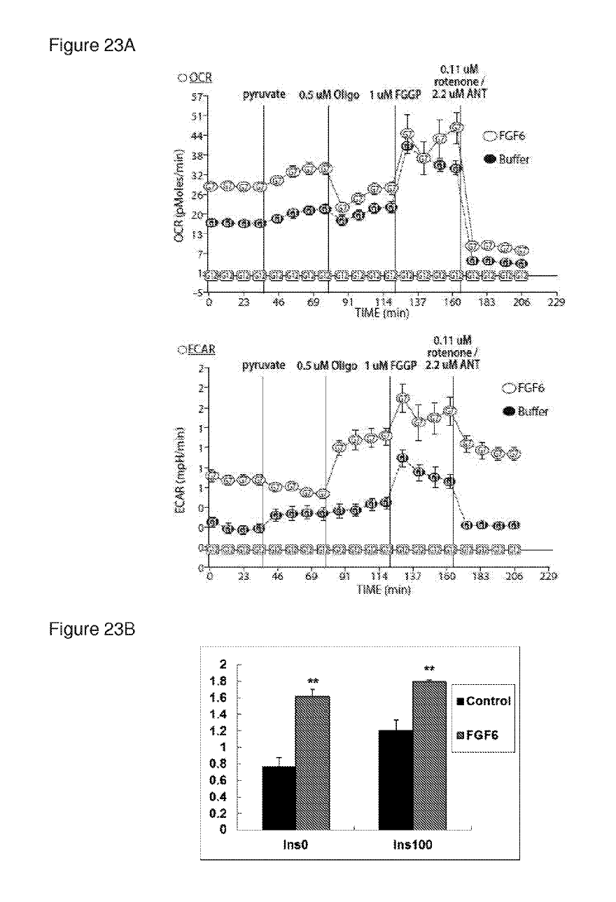

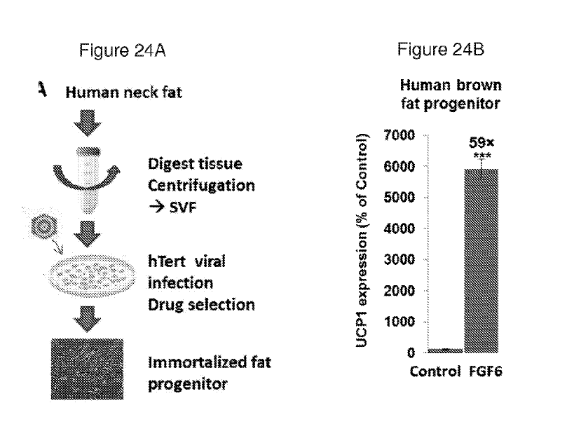

The effect of FGF6 on murine mature brown adipocytes was also examined. Mature brown adipocytes were treated with FGF6 in growth media (DMEM+10% FBS), a vehicle control (buffer) (“buffer” referred to in FIG. 22), or BMP7 for seven days. Following the treatment, cells were examined for the color of the culture media in combination with oil Red O staining, expression of adipogenic markers, and expression of brown fat markers.

mRNA expression of UCP1, PPARγ, PTGS2, NDST3 and SIRT1 was measured in the mature adipocytes exposed to either the control, BMP7, or FGF6. The results from this analysis are provided in FIG. 22. The results show that unlike in preadipocytes, FGF6 does not induce the expression of UCP1 in mature adipocytes to the extent that it was induced by the control buffer or BMP7. Using cell staining with oil red O, it was also determined that there was no acidification of cell culture media—evidenced by a lack of increased staining with oil red O.

In contrast, addition of FGF6 to mature brown adipocytes had a similar effect on cellular oxygen consumption and glucose uptake as in preadipocytes. To assess mitochondrial respiration, a Seahorse Extracellular Flux Analyzer (Seahorse Bioscience Inc., North Billerica, Mass.) was used to quantify oxygen consumption rates (OCR) of differentiated human brown adipocytes. Wt-1 brown preadipocyte cells were seeded on 24-well format plates and allowed to adhere overnight. After adipogenic induction for 8 days, OCR was analyzed. To measure OCR independent of oxidative phosphorylation, 0.5 μM oligomycin (EMD Chemicals Inc., Gibbstown, N.J.) was added to cells. Subsequently, 0.8 μM FCCP (carbonyl cyanide-p-trifluoromethoxyphenylhydrazone) and 1 μM respiratory chain inhibitors (rotenone) were added to measure maximal respiration and basal rates of nonmitochondrial respiration. As shown in FIGS. 23A and 23B, treatment of FGF6 enhanced oxygen consumption and glucose uptake in mature brown adipocytes. Thus, FGF6 was able to activate the mature brown adipocyte's ability to use glucose and increase energy expenditure without inducing UCP-1.

### Example 14: FGF6 Upregulates UCP1 Expression in Immortalized Human Adipocytes

The role of FGF6 in regulating UCP1 expression and mitochondrial activity was studied using murine committed preadipocytes, as well as primary cultures isolated from different adipose depots of mice. In order to confirm the effect of FGF6 on UCP1 in human adipocytes, immortalized human brown and white fat precursor cells were generated.

Human preadipocyte pooled cell populations derived from a total of four human subjects were generated by isolating cells from the stromal vascular fraction (SVF) of human neck fat and immortalizing them via stable expression of human telomere reverse transcriptase (hTert) (process is described in FIG. 24A). Pairs of immortalized progenitors for human BAT (hBAT-SVF, isolated from deep neck fat) and human WAT (hWAT-SVF, isolated from superficial neck fat) of the same individuals were established from each of the four individuals for proper comparisons. The immortalized cells were passaged in culture for more than 90 days and were followed for at least 20 population doublings. The immortalized cells retained morphological and differentiation characteristics of primary cells.

The effect of FGF6 on UCP1 expression in human brown fat progenitor cells was studied as described above. FGF6 could induce a nearly 60-fold increase in UCP1 expression in human brown fat precursors, as described in FIG. 24B. This data confirms the role of FGF6 in upregulating UCP1 expression in human progenitor cells.

### Example 15: Prostaglandins Induce UCP1 Expression and Enhance Oxygen Consumption and Glucose Uptake in Preadipocytes

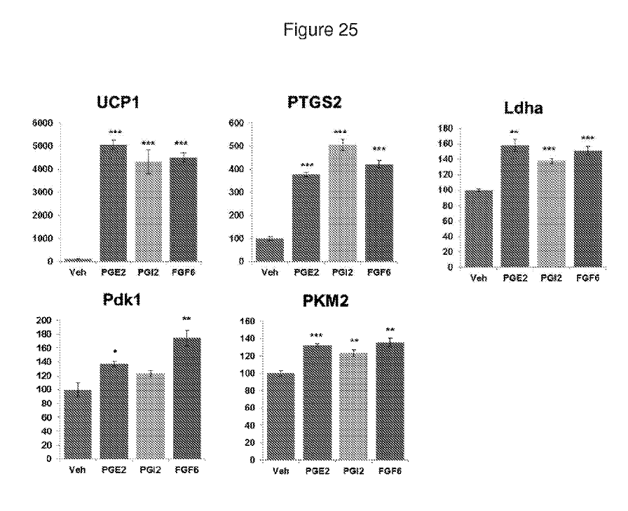

To test the role of specific prostaglandins (PGs) in activating UCP1 expression, WT-1 brown adipocytes were pretreated with PGE2, PGI2 and FGF6 for 24 hours. mRNA expression levels of UCP1, PTGS2, LDHA, PDK1 and PKM2 were then measured. As described in FIG. 25, treatment of murine brown preadipocyte WT-1 cells with FGF6, PGE2 and PGI2 each resulted in an increase in UCP1, PTGS2, LDHA, PDK1 and PKM2 expression relative to the vehicle control (Veh) (buffer alone), in FIG. 25). The data indicate that compared to vehicle group, FGF6 as well as PGE2 and PGI2 significantly induced the expression of UCP1 and PTGS2.

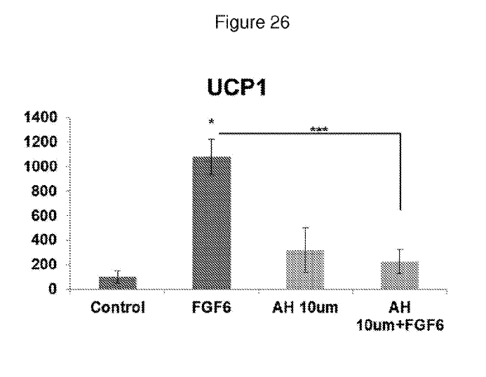

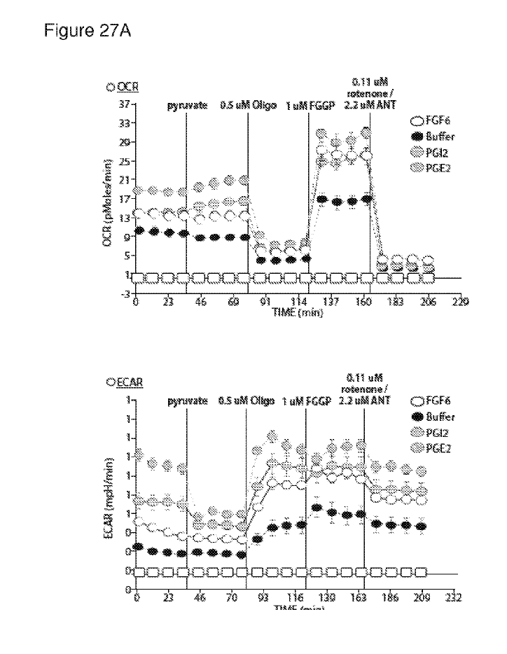

The role of the PGE2-EP4 receptor was also examined. WT-1 brown adipocytes were pretreated with AH-23848 (Sigma Aldrich), a calcium salt which is an inhibitor of the PGE2-EP4 receptor. Following AH-23848 (“AH”) treatment of the WT-1 cells at concentrations of 0 uM and 10 uM, the cells were treated with FGF6 alone in growth media (DMEM+10% FBS), AH alone, or the combination of FGF6 and AH for 24 hours. UCP1 mRNA expression for each group was then determined. As demonstrated in FIG. 26, the effect of FGF6 on UCP1 expression was reduced in the presence of AH, further suggesting a role for the prostaglandins (PGs) in activating UCP1 expression.

The role of PGs in regulating mitochondrial function and cellular glucose uptake was also assessed. WT-1 brown preadipocytes were treated with PGE2, PGI2 and FGF6 in growth media (DMEM+10% FBS) for two days. Similar to treatment with FGF6, treatment with PGE2 and PGI2 enhanced oxygen consumption and glucose uptake, as described in FIGS. 27A and 27B, respectively.

### Example 16: FGF6 Regulates UCP1 Induction Via FGFR1, and Inhibition of Sirt1 Reduces the Effect of FGF6 on UCP1 Expression

To determine the signaling pathway(s) by which FGF6 regulates UCP1 expression and mitochondrial activity, an experiment was performed to identify what receptor(s) FGF6 may be acting through in preadipocytes to regulate UCP1 expression.

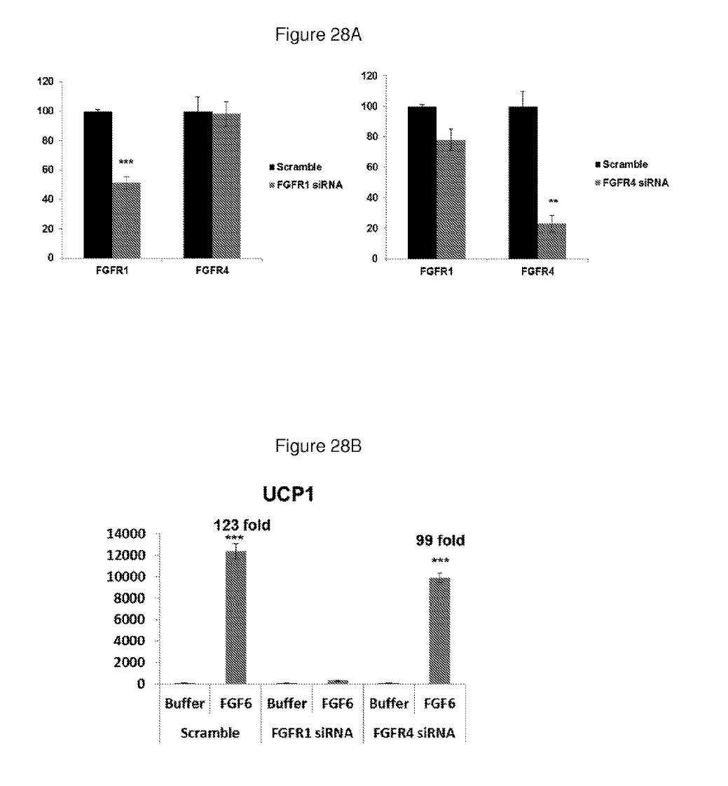

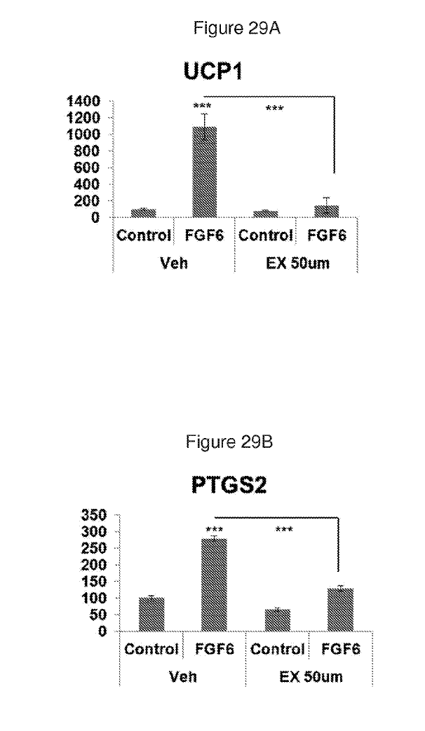

It has been reported that FGF6 transduces signals into cells preferentially via FGFR1 and FGFR4 (Ornitz, et al., J Biol Chem 271:15292-15297, 1996). In order to determine whether FGFR1 or FGFR4 were acting as an FGF6 receptor(s) in UCP1 expression, specific siRNA for FGFR1 and FGFR4 was used to knockdown their expression in preadipocytes. As described in FIG. 28A, the FGFR1 siRNA was specific in decreasing expression of FGFR1 and not affecting expression of FGFR4, and vice versa (expression was determined using QPCR). The negative control siRNA (“scramble”) described in FIG. 28A had no impact on either FGFR1 or FGFR4 expression.

Subsequently UCP1 expression levels were determined upon treatment of cells with FGF6, where the cells were also exposed to either FGFR1 or FGFR4 siRNA. As demonstrated in FIG. 28B, addition of FGFR1-specific siRNA abolished the effect of FGF6 on UCP1 induction, whereas siRNA targeting FGFR4 or the negative scramble control siRNA had no substantial effect on UCP1 expression. These data suggest that FGF6 induces UCP1 expression by specific activation of FGFR1, but not via FGFR4.

To determine if the AMPK pathway is utilized by FGF6 to regulate UCP1 expression and mitochondrial function, WT-1 preadipocytes were pretreated with the Sirt2 selective inhibitor EX (EX 527; Santa Cruz Biotechnology) at concentrations of 0 uM or 50 uM in growth media (DMEM+10% FBS) for 3 hours. Following EX treatment, the cells were treated with EX and FGF6 for 18 hours.

UCP1 mRNA expression was determined. Addition of the Sirt2 inhibitor inhibited UCP1 expression, as described in FIG. 29A. Similarly, the expression level of PTGS2 was also reduced upon treatment of EX, as described in FIG. 29B. This data suggests that the AMPK pathways mediates, at least in part, the effect of FGF6 on UCP1 mRNA induction.

### Example 17: FGF6 Induces UCP1 Expression In Vivo and Improves Glucose Tolerance in DIO Mice

To determine whether FGF6 is able to induce UCP1 expression in vivo, UCP1 reporter mice and lentiviral-mediated gene transfer were utilized. The UCP1 reporter mice (UCP1-cre/LUC) were generated by crossing UCP1-cre mice with the Rosa-Luciferase reporter strain. In this model, cells that express UCP1 during their life cycle will permanently express luciferase. Luciferase activity in the UCP1-cre/LUC mouse can be monitored in vivo when the luciferase substrate luciferin is injected into the reporter mouse. Because the sqWAT depot expresses little to no UCP1 at basal state, but UCP1 transcription can be robustly turned on in response to different stimuli, it serves as an ideal site for testing the effect of molecules that may increase UCP1 gene expression in this reporter model.

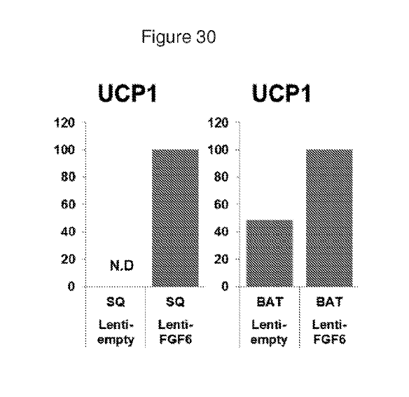

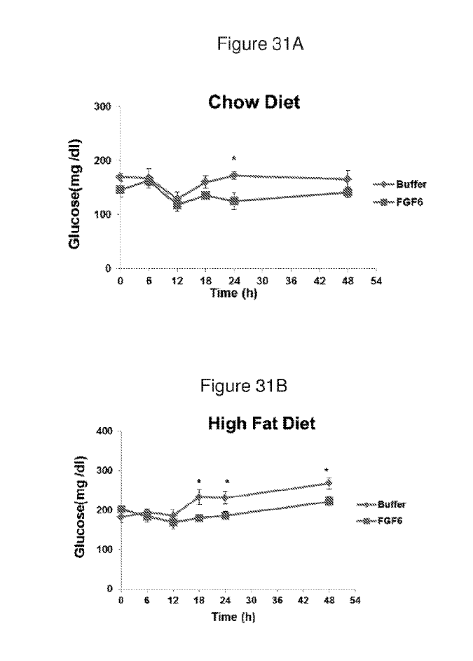

Lentivirus expressing FGF6 (Lenti-FGF6) or control virus (Lenti-Crl) was injected into left and right subcutaneous white adipose tissue (SQ) of a UCP1-cre/LUC mouse, respectively. The right subcutaneous white adipose tissue (SQ) depot receiving lenti-FGF6 displayed high level of luciferase activity compare with the sqWAT on the left side receiving lenti-Crl. Similarly, brown adipose tissue (BAT) receiving lenti-FGF6 also displayed high levels of luciferase activity compared with BAT receiving lenti-Crl, as quantitated by QPCR (see FIG. 30). These data suggest that FGF6 administration was able to induce UCP1 gene expression in vivo.

To determine whether induction of UCP1 and mitochondrial activity by FGF6 could lead to increased nutrient utilization and lower blood glucose or fatty acid in obese mice, diet-induced obese (DIO) mice were treated with recombinant FGF6, and glucose levels were monitored after injection. Specifically, C57BL6 mice (11 weeks) were fed on either a high fat diet (HFD) or a Chow diet (a regular animal diet as a control) and were injected subcutaneously with 0.5 mg/kg recombinant FGF6 (n=5) or buffer (n=5). Glucose levels were measured every 6 hours after injection. Obese FGF6-treated mice (HFD mice) showed a lower glucose level when compared with control mice (buffer injected), as described in FIG. 31. Chow diet fed mice also showed a reduction in glucose levels (as described in FIG. 31A). Thus, the FGF6-injected HFD mice showed a higher level of glucose tolerance relative to HFD mice who received the negative control.

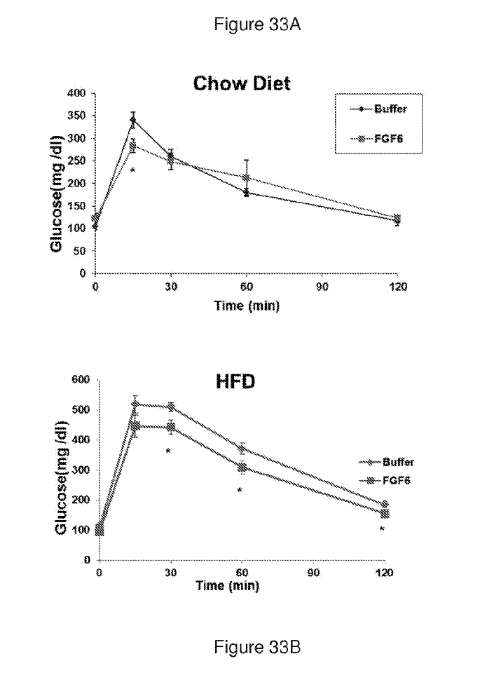

Furthermore, the glucose tolerance test described in FIGS. 33A and 33B showed that FGF6 can be used as a treatment to improve glucose tolerance for patients having obesity, represented by the obese mouse model. C57BL6 mice were fed either the Chow diet or a high fat diet (HFD) and were fasted overnight (16 h) prior to intraperitoneal injection of 2 mg/g body weight of glucose using a 20% (w/v) solution. Blood glucose measurements were conducted before and 15, 30, 60, and 120 min after injection. where it was determined that FGF6 enhanced glucose tolerance, particularly in obese HFD mice (as described in FIGS. 33A and 33B).

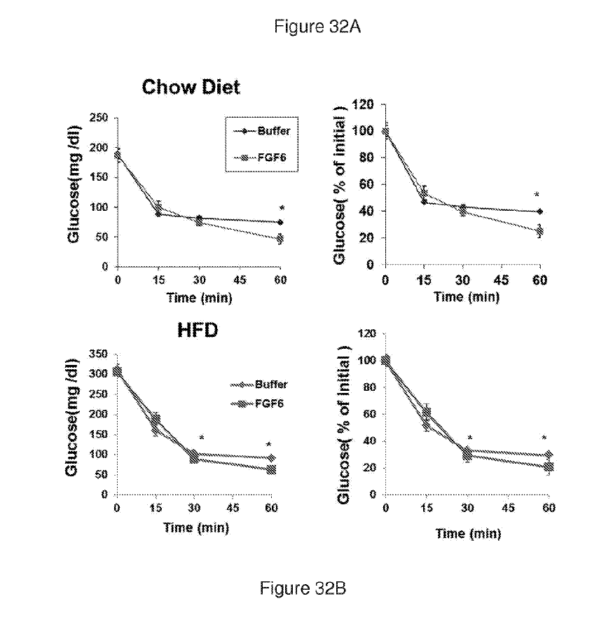

In addition to testing the impact of FGF6 on glucose tolerance, an insulin tolerance test was also performed using Chow fed and Obese (HFD) mice. Animals were fasted for two hours before receiving an intraperitoneal dose of 1.5 IU of recombinant human insulin (Humalog; Eli Lilly and Company, Indianapolis, Ind.). Blood samples were collected from the tail vein for measurement of blood glucose levels using a glucometer before and 15, 30, and 60 min after injections of FGF6 and the buffer control. As described in FIGS. 32A and 32B, subcutaneous injection of 0.5 mg/kg of FGF6 and 1.5 U of insulin per kg body weight into C57BL6 mice fed either the Chow diet of a high fat diet resulted in lower levels of glucose in the FGF6 injected mice, suggesting that FGF6 was able to increase insulin sensitivity.

Obese FGF6-treated mice exhibited enhanced insulin sensitivity and improved glucose tolerance compared with control mice (who received buffer alone), as shown in FIGS. 32 and 33, respectively. These data suggest that induction of UCP1 by FGF6 leads to increased nutrient utilization, and thus FGF6 can lower blood glucose in obese mice.

### EQUIVALENTS

Those skilled in the art will recognize, or be able to ascertain using no more than routine experimentation, many equivalents to the specific embodiments of the invention described herein. Such equivalents are intended to be encompassed by the following claims. The contents of all references, patents and published patent applications cited throughout this application are incorporated herein by reference.

1. A method of expressing uncoupling protein 1 (UCP1) in an FGF-receptive cell, said method comprising contacting the FGF-receptive cell with an FGF receptor agonist, in an amount sufficient to induce UCP1 expression, such that UCP1 is expressed in the FGF-receptive cell, wherein the FGF-receptive cell does not exhibit substantial lipid accumulation and does not differentiate into a brown adipocyte following contact with the FGF receptor agonist.

2. (canceled)

3. The method of claim 1, wherein the FGF-receptive cell is an undifferentiated cell selected from the group consisting of a primary adipose precursor, an adult stem cell, an embryonic stem cell, an induced pluripotent stem cell, a stromal-vascular fraction cell, an immortalized human brown fat precursor cell, an immortalized human white fat precursor cell, a brown preadipocyte, and a white preadipocyte.

4. (canceled)

5. The method of claim 1, wherein the FGF receptor agonist is selected from the group consisting of an FGF protein (or functional fragment thereof), a nucleic acid encoding an FGF protein (or functional fragment thereof), an FGF mimetic, and an anti-FGF receptor agonist antibody, or an antigen-binding fragment thereof.

6. The method of claim 5, wherein the FGF protein is not FGF21.

7. The method of claim 5, wherein the FGF protein is selected from the group consisting of FGF1, FGF2, FGF4, FGF6, FGF8, FGF9, FGF16, FGF17, FGF18, and FGF20.

8-15. (canceled)

16. The method of claim 1, wherein the FGF-receptive cell does not exhibit substantial increases in expression of a brown adipocyte marker selected from the group consisting of PR Domain Containing 16 (PRDM16), PPAR-gamma Coactivator 1 (PGC1), Adipocyte Protein 2 (Ap2), and Cell Death Inducing DFFA-Like Effector A (CIDEA).

17. A method of treating a subject having a disorder that would benefit from metabolic control, said method comprising administering a composition comprising an FGF receptor agonist to the subject, such that the disorder is treated, wherein the FGF receptor agonist is administered to the subject in the absence of an additional agent selected from the group consisting of an additional growth factor, dexamethasone, and indomethacin.

18. The method of claim 17, wherein the FGF receptor agonist is administered to the subject by subcutaneous injection.

19. (canceled)

20. The method of claim 17, wherein the FGF receptor agonist is a nucleic acid encoding an FGF protein and is administered to the subject via a viral vector.

21. The method of claim 17, wherein the FGF receptor agonist is administered to the subject via a drug delivery matrix.

22. The method of claim 21, wherein the drug delivery matrix is silk hydrogel.

23. The method of claim 17, wherein
the FGF receptor agonist is administered to adipose tissue of the subject.

24. (canceled)

25. The method of claim 17, wherein the disorder is selected from the group consisting of a disease that would benefit from glucose control, a disease that would benefit from weight control, a disease that would benefit from cholesterol control, and a fatty acid metabolism disorder.

26. The method of claim 25, wherein the disease that would benefit from glucose control is selected from the group consisting of insulin resistance, diabetes, and hyperglycemia, wherein the disease that would benefit from weight control is selected from the group consisting of liver disease, dyslipidemia, a glycemic control disorder, cardiovascular disease and obesity, and wherein the disease that would benefit from cholesterol control is heart disease.

27. The method of claim 25, wherein the disorder is diabetes or obesity, and wherein the FGF receptor agonist is FGF6 protein or a nucleic acid encoding an FGF6 protein.

28. The method of claim 25, wherein the disorder is metabolic syndrome.

29. The method of claim 28, wherein the FGF receptor agonist is FGF6 protein or a nucleic acid encoding an FGF6 protein.

30. The method of claim 28, wherein the subject has insulin resistance and/or insulin insensitivity.

31. (canceled)

32. The method of claim 17, wherein the FGF receptor agonist is selected from the group consisting of an FGF protein (or functional fragment thereof), a nucleic acid encoding an FGF protein (or functional fragment thereof), an FGF mimetic, and an anti-FGF receptor agonist antibody, or an antigen-binding fragment thereof.

33. The method of claim 32, wherein the FGF protein is not FGF21.

34. The method of claim 32, wherein the FGF protein is selected from the group consisting of FGF1, FGF2, FGF4, FGF6, FGF8, FGF9, FGF16, FGF17, FGF18, and FGF20.

35. (canceled)

36. The method of claim 17, wherein the FGF receptor agonist is administered at a dose of about 0.5 mg/kg to about 300 mg/kg.

37. (canceled)

38. An ex vivo method of treating a subject having a disorder that would benefit from metabolic control, said method comprising administering an FGF-receptive cell contacted with an FGF receptor agonist to the subject, such that the disorder is treated, wherein the FGF-receptive cell is administered to the subject in the absence of an additional agent selected from the group consisting of an additional growth factor, dexamethasone, and indomethacin.

39-40. (canceled)

41. The method of claim 38, wherein the disorder is selected from the group consisting of a disease that would benefit from glucose control, a disease that would benefit from weight control, a disease that would benefit from cholesterol control, and a fatty acid metabolism disorder.

42. The method of claim 41, wherein the disease that would benefit from glucose control is selected from the group consisting of insulin resistance, diabetes, and hyperglycemia, wherein the disease that would benefit from weight control is selected from the group consisting of liver disease, dyslipidemia, a glycemic control disorder, cardiovascular disease and obesity, and wherein the disease that would benefit from cholesterol control is heart disease.

43. The method of claim 41, wherein the disorder is diabetes or obesity, and wherein the FGF receptor agonist is FGF6 protein or a nucleic acid encoding an FGF6 protein is administered to the subject by injection.

44. The method of claim 41, wherein the disorder is metabolic syndrome.

45. The method of claim 44, wherein
the FGF receptor agonist is FGF6 protein or a nucleic acid encoding an FGF6 protein.

46. The method of claim 44, wherein the subject has insulin resistance and/or insulin insensitivity.

47. The method of claim 38, wherein the FGF receptor agonist is selected from the group consisting of an FGF protein (or functional fragment thereof), a nucleic acid encoding an FGF protein (or functional fragment thereof), an FGF mimetic, and an anti-FGF receptor agonist antibody, or an antigen-binding fragment thereof.

48. The method of claim 38, wherein the FGF-receptive cell is administered to adipose tissue of the subject.

49. The method of claim 27, wherein an anti-FGFR1 agonist antibody is administered to the subject.

50. The method of claim 38, wherein the subject is human.

51. A method for lowering the weight of a subject, said method comprising
selecting a subject in need of weight loss, and
locally administering to white adipose tissue of the subject an FGF receptor agonist, thereby lowering the weight of the subject.

52-57. (canceled)

58. The method of claim 51, wherein the subject is human.

59. The method of claim 51, wherein the FGF receptor agonist is selected from the group consisting of an FGF protein (or functional fragment thereof), a nucleic acid encoding an FGF protein (or functional fragment thereof), an FGF mimetic, and an anti-FGF receptor agonist antibody, or an antigen-binding fragment thereof.

60-63. (canceled)


SEQUENCE LISTING (56 sequences)

SEQ ID NO: 1 - DNA, 744 bp, Homo sapiens
tttagggccattaattctgaccacgtgcctgagaggcaaggtggatggccctgggacagaaactgttcatcactatgtcccggggagcaggacgtctgcagggcacgctgtgggctctcgtcttcctaggcatcctagtgggcatggtggtgccctcgcctgcaggcacccgtgccaacaacacgctgctggactcgaggggctggggcaccctgctgtccaggtctcgcgcggggctagctggagagattgccggggtgaactgggaaagtggctatttggtggggatcaagcggcagcggaggctctactgcaacgtgggcatcggctttcacctccaggtgctccccgacggccggatcagcgggacccacgaggagaacccctacagcctgctggaaatttccactgtggagcgaggcgtggtgagtctctttggagtgagaagtgccctcttcgttgccatgaacagtaaaggaagattgtacgcaacgcccagcttccaagaagaatgcaagttcagagaaaccctcctgcccaacaattacaatgcctacgagtcagacttgtaccaagggacctacattgccctgagcaaatacggacgggtaaagcggggcagcaaggtgtccccgatcatgactgtcactcatttccttcccaggatctaaggacccacaaaagaaggcttacagatttaaagcatcatctgttcgattgaaattttgcaccagcgaagaattc

SEQ ID NO: 2 - protein, 208 aa, Homo sapiens
MALGQKLFITMSRGAGRLQGTLWALVFLGILVGMVVPSPAGTRANNTLLDSRGWGTLLSRSRAGLAGEIAGVNWESGYLVGIKRQRRLYCNVGIGFHLQVLPDGRISGTHEENPYSLLEISTVERGVVSLFGVRSALFVAMNSKGRLYATPSFQEECKFRETLLPNNYNAYESDLYQGTYIALSKYGRVKRGSKVSPIMTVTHFLPRI

SEQ ID NO: 3 - DNA, 1232 bp, Mus musculus
tttagggccattaattctgaccacgtgcctgagaggcaaggtggatggccctgggacagaggctgttcatcactatgtcccggggagcaggacgtgttcagggcacgctgcaggctctcgtcttcttaggcgtcctagtgggcatggtggtgccctcacctgccggcgcccgcgccaacggcacgctactggactccagaggctggggcaccctcttgtccaggtctcgagctgggctagctggagagatttcgggtgtgaattgggaaagcggctatttggtgggcattaagcgacagcggagactctactgcaacgtgggcattggcttccacctccaggtgcctcctgacggccgcatcagtggaacacacgaggagaacccctacagcctgctggagatctccacggtagaacggggtgtggtgagcctcttcggggtgaagagtgctctcttcattgccatgaacagtaaaggaagactgtacacaacgcccagcttccacgacgaatgcaagttccgagaaaccctccttccaaacaactacaacgcctacgagtcagacctgtacagagggacctacattgcactgagcaaatatggacgggtaaagcggggcagcaaggtgtccccaatcatgactgtcactcacttcctccccaggatataaggacccatggatgaaggctcacggggtgacagctgcgtcttttctgtcaggattttacactaagagaacgcttctgctcctatcccgtgacttctgagtttggcttatctcggcctgaagatctgagtggatgccctgggccactggactgcatggtgtgtaagccacgtggctgagggtctacagttcacatttcaggatggcaggggctcctgtgcttcagcctcacctgaggagagacaatacagatgactcttgcagcgtgtggccctgtgcataagaaaaagccaaaatgggagtatgcatgtgtccgtgcatatccacacatctattcgtagattctcagagcaaaggcattttaggataccgagttcctttctcccaacatcgtcaccgcaggaaactgcctgtcagagcgtcacttgtcgagtcagcaggattgagatgaaccccattctgccgttttctgatcatctatctatccagttgtctgtgtaacagtgagcagctatcttttatgtgccaggaggaaggtaggggtataaaatataactgtaaact

SEQ ID NO: 4 - protein, 208 aa, Mus musculus
MALGQRLFITMSRGAGRVQGTLQALVFLGVLVGMVVPSPAGARANGTLLDSRGWGTLLSRSRAGLAGEISGVNWESGYLVGIKRQRRLYCNVGIGFHLQVPPDGRISGTHEENPYSLLEISTVERGVVSLFGVKSALFIAMNSKGRLYTTPSFHDECKFRETLLPNNYNAYESDLYRGTYIALSKYGRVKRGSKVSPIMTVTHFLPRI

SEQ ID NO: 5 - DNA, 4545 bp, Homo sapiens
actctgcgcgccggcgggggctgcgcaggaggagcgctccgcccggctacaacgctccgcgagccggcgcggcaacacctgttcgcggcagcctgggcggcacgcgagctcccggacgcggctctcctcgctcgccgctcgccacccgttctaagccaatggacatctgccgagcctctggagaatcctggatactagctttggacgcctaaagtttcttcttctttttgttttattattattatcattttttggaggggggaccgggaggggagatttgtcgccgccaccaacgtgagatttttttttccccttgaaggattcatgctgatgtctgcagagtcggttagagagtaaaaacagcgcatgccttcctggagtcaggatccgtaaattctgacgtagcccgtgcatcttaaaaatccctataataacgcctaggcatttaagttgctatggtcattctgatctcaaaccaaatggagaaactacggattttttttccttattacggtcggatgggatgaagaccttcctgcctgctaagagctggggatctatctatagagatacatagatatgtttatcaatatgtcagtgtgtgagtataaagtggtggtttcttagactatcagtggtttgaccttgaacctgtgccagtgaaacagcagattacttttatttatgcatttaatggattgaagaaaagaaccttttttttctctctctctctgcaactgcagtaagggaggggagttggatatacctcgcctaatatctcctgggttgacaccatcattattgtttattcttgtgctccaaaagccgagtcctctgatggctcccttaggtgaagttgggaactatttcggtgtgcaggatgcggtaccgtttgggaatgtgcccgtgttgccggtggacagcccggttttgttaagtgaccacctgggtcagtccgaagcaggggggctccccaggggacccgcagtcacggacttggatcatttaaaggggattctcaggcggaggcagctatactgcaggactggatttcacttagaaatcttccccaatggtactatccagggaaccaggaaagaccacagccgatttggcattctggaatttatcagtatagcagtgggcctggtcagcattcgaggcgtggacagtggactctacctcgggatgaatgagaagggggagctgtatggatcagaaaaactaacccaagagtgtgtattcagagaacagttcgaagaaaactggtataatacgtactcatcaaacctatataagcacgtggacactggaaggcgatactatgttgcattaaataaagatgggaccccgagagaagggactaggactaaacggcaccagaaattcacacattttttacctagaccagtggaccccgacaaagtacctgaactgtataaggatattctaagccaaagttgacaaagacagtttcttcacttgagcccttaaaaaagtaaccactataaaggtttcacgcggtgggttcttattgattcgctgtgtcatcacatcagctccactgttgccaaactttgtcgcatgcataatgtatgatggaggcttggatgggaatatgctgattttgttctgcacttaaaggcttctcctcctggagggctgcctagggccacttgcttgatttatcatgagagaagaggagagagagagagactgagcgctaggagtgtgtgtatgtgtgtgtgtgtgtgtgtgtgtgtgtgtgtatgtgtgtagcgggagatgtgggcggagcgagagcaaaaggactgcggcctgatgcatgctggaaaaagacacgcttttcatttctgatcagttgtacttcatcctatatcagcacagctgccatacttcgacttatcaggattctggctggtggcctgcgcgagggtgcagtcttacttaaaagactttcagttaattctcactggtatcatcgcagtgaacttaaagcaaagacctcttagtaaaaaataaaaaaaaataaaaaataaaaataaaaaaagttaaatttatttatagaaattccaaaggcaacattttatttattttatatatttatttattatatagagtttatttttaatgaaacatgtacaggccagataggcattttggaagctttaggctctgtaagcattaaatggcaaagtccgctatgaacctgtggtaaattcatgcaagtagatataatggtgcatggatataagaaattctaatgaccctaatgtactaaaggcgacaatctcttttgtgcccatattattgtaaacttatgcacatcgctcatgacactgagtattcactcttcagactgcttgtttcatagcttatcccagaggattaaagataaactgggtctcaaactttgattctgtgtctgcaatatttcctctctcataagtgactccactattgtaacttcatggttggaaaatatgagggttgatatatgtcttacttgtttaaatctgtcgcagaatataccaaagctaaataataactatgctttcattttagccgatctccagaatgacagtattaacatcaaacattgtattgatttagaattctcaaaaaaggaaaaaaaagtacatagcacagactattttttttaaagacgtaagaatcagattaacaggatcatacttgtaaactttttttggttcacttggctatcaaatatgaaattatagaagtatcataggggtcattgtaacatcttttagagaaaatggctatcagtgtgaactgtcataattacgtggtaatagcacccttagtaaaacttgcaaaatgaaactaataaatcgttatcaataatgacaatgagggggaaagtattatacttgttgactgtgttttgttttttaaaatggtctccacaagcgctcaatttttttagaggggatattactatatagaatatcttttacaaggcttttataacattttatgctgaaaagcataagaatacgtatttctttagtagcaataattttggaacttgcccttgggcaagcgagactatttcttactatatactaaggagaaaagagccaaattcttaaagcaatatttaagaaaaaaggaatttataacaaattctcatctacatatgacactttctagccagttgtgttgagaagtgcaaagtgacggtttaaacatgtgttgggatttattgaactaattttaaaatttactattcaaactttattttgctctgatgcacattctctatgaaaaataaaagtgtgtcactggtgagtgacagctgttatgagctagaagcgcatgacttattgtgacgatgtcttgcctttctgtggtccaagttggagtacatggcaatgccctcctgctgatgtgcattaaggaaaatctaagtctaatatttggaattaagatatattttagggggaggggacagaagcaatgtaaaatagttgatttatgataaagctcagaatgtcctcttcatttattttcttgttttattttcctttctaaacagaaactgcatttaattccaaaaagtagtattcttatttattatttaaccctttgctgctgctaaaatgtgcacatattcaggctttagtttttccaaaaggcattttttttttggctgaaaaatattaaacatttgaccacagggaagaatcaagtttctaggatgtcataggtatactatgtagcactgaaaaaattgattttaggtgacagccaaaagtagtcttaaagtagcatgagaccttagataatcgacctaaaagaaagaaaattgtgaaaaagacaaaaatcttcatgcattcctataaaacgctactttaaggtctacttttggagttaattttgtttggtactttttttttttttaagacgagcaaattgttatatgcttttggcaattgatacaataaactgtaatggtctgtaaataaataaatattgactcatgcgatttatgtaaatagtggaactgggagagtggatggctcagggtttcggtgtgggcattgtctcttgggcagtagagtgagtcatccccagctcatgggtttgcatccagttcttgtcttaagagacccaaagcccagtgaatggcagccctgagccactgtggaatgggggttctggtttcacaaacagatgcttagatagccaaaccactgtcttgttggtgccaacacttgcactgtggtcaaagacttaccgagcatgggctgaacaaccttcccatctgtcatgtgaatgtccccaagcagtggtgaaggacatgctaggtcagtgttggggaacctgccctgccaggtcctgttttgtagataaacaaatggctgccttctggtgtttttattctatttcatctcattaacactacaaccttgtgttatttacttgataatctgtaattgtatgtaaatacatacaggattatgtaatttgtgtaaatacataattacagagttttgaaaactgaaaaaaaaaaaaaaa

SEQ ID NO: 6 - protein, 208 aa, Homo sapiens
MAPLGEVGNYFGVQDAVPFGNVPVLPVDSPVLLSDHLGQSEAGGLPRGPAVTDLDHLKGILRRRQLYCRTGFHLEIFPNGTIQGTRKDHSRFGILEFISIAVGLVSIRGVDSGLYLGMNEKGELYGSEKLTQECVFREQFEENWYNTYSSNLYKHVDTGRRYYVALNKDGTPREGTRTKRHQKFTHFLPRPVDPDKVPELYKDILSQS

SEQ ID NO: 7 - DNA, 4145 bp, Mus musculus
attctgatctctaagcaaatggagaaactacggatttttttcccttattacggtcggatgggatgaagacctttctgcctgctgagagtcgaggctccatatgtagcgatgcatagctgtgttgatcaatgtcagtgtgtgagtataaagtggtggcttcttagactatcagtggtttgaccttgaacctgtgccagagaaacagccgattacttttatttatgcatcggatggattgaagaaaagaaccctttttccctctctgtctgcaactgcggcaagggaggggagttggatatacctcgcctagtgtctcctggttgataccatcattattgtttattcttgtgcttaaaagccgagtcctctgatggctcccttaggtgaagttgggagctatttcggtgtgcaggacgcggtaccgttcgggaacgtaccggtgttgccggtggacagtccggtgttgctaagtgaccacctgggtcagtccgaagcaggggggctgccccggggccccgcagtcacggacttggatcatttaaaggggattctcaggcggaggcagctgtactgcaggactggatttcatttagagatcttccccaacggtactatccagggaaccaggaaagaccacagccgcttcggcattctggaatttatcagtatagcagtgggcctggtcagcattcgcggtgtggacagtggactctacctcggcatgaacgagaagggggagctgtatggatcagaaaaactaacacaggaatgtgtgttcagagaacagtttgaagagaactggtacaacacctactcttccaacctctataaacatgtggacaccggaaggagatactatgttgcattaaataaggacgggactccaagagaagggaccaggactaaacggcaccagaaatttacacattttttacctagaccagtggaccctgacaaagtacctgaactatataaggatattctaagccaaagctgacaaagacagtgtcttcacttgagcccttaaaacataaccactataaatgctttcatgcggtgggttcttattgattagcagtgccgtcacctcagctccactgttgccaaactttgtcgcatgcatatgaatgatggaagcttggatgaggacttgccaatttgctctgcacttactggctggtcctcctggagggctgcctagggccacttgcttgatttattacaaaagcaggggagagaggctgcgggagagggtgcgagtgcatgagtgagtggaggggactgcagcgcgccacgaggacaatggcctgatgcatgctgggaaatagacacgcttttacatttttgatcagttgaacttcattctatatcagcacagctgccatacttcaactcatcaggatttttggctggtggcctgctcaagggaacactgcctttccacaggcgtggagagagcagtctcacttaaaaagactatcagttcattcacactggtatcatagcccagtgaatttaaagcaaagacctcttagtttaaaagaaaaaaaaaaaaagaaagaaaaaaaagaaaagaaaaagaaaaaaacaaggaaaaaaagaaaaaaaagaaaaaaggttaaatttatttatagaaattccaaaggcaacattttatttattttatatatttatttattatatagagtttatttttaatgaaatatgtacaggccagataggcattttggaagctttaggctctgtaggcattaaatggcaaagtctgctatgaacctgtggtaacttcatgcaagtaggtatacgagaaattctaatggccctaatgtactaaaggcgacaatctattttgtgcccatattattgtaaacttctgcacatcgctcatgacactgaggagtcactcttcagactgcttgtttcatagcttatcccagaggattaaagataaactgggtttcaacccttcatttcgtgtctgtaattttcctctctcgtaattcagtctctcagtgtaactgggcggttggaacatatgagggttggtgtatgtcttacttgttttaatctattgcagagtgtaccaaagctaagcaatgactatgcgttttaaatttcagcccacttctgtgacaacagggctaacatcaaacatggcactgattaaaaattcccaagggagaggaaaatccatttacatagctcctcctcctcttcctccgcctcttcctcctcttcctcttcgacttcctcggcctcctcttcttttctttcttttcttttcttttcttttcttttcttttcttttcttttcttttcttttcttttcttttcttttcttttcttttcttttctttcttttcttttcttttctcttctcttctcttctcttcttttcttttcttttcttttcttttcttttctttccttttcttttcttttctgaggcaaaaattagatgaccgggatcattcttgtaaacattttatttgtgtcttggctatcaaatatgaaattctacatggaccctagggctcactataacatttttaatggagctggcatttggtgtgaactgtcataatcacatggggttagtaacctccaagttaaactggcaacatggaactaatgtcttcatcagtaacgaccttggaggggttactgtagccattgcctgtgcttcatctccgaatcggacttcagcagttctcagagaagtgggtactaaatagaacactgtttacaaaaccttttataacattatatgctgaaaagcccaagaatacatacttatttgatagccaaaatcctcctgttgggtaagggagactatttctctctcactatataccaaggtgaaaagagccaagttcttgaagcaataaagaaatctataacacattctcactctctagctcgttgatgtggggaaattgaaagtgacagctaaaaaaatatgtttgaatttgtgcagctaattttaagatgcaatattctggcttcactttgcttggatgcacattctcgatgggaagcgaaagcgtgtcactggcaagggatagctgttgtggtctagaaatgcctgtcttcctgtcctgagcaaatccatgtagccatcttatggtccaagctggagcgggtgacgatcttccgtgctgatgagcattagggaggaacttgtatttggaactgagagatgtcacagagagaggagccagacgctctgtggatcaggcaataggataaagatcaggatgcattctgcatgtgtgttctgtggatcccccacccgctttctaaacaggagccggcgtgaattttgagcagtgtttttgtttgttatttaacactttgctgctaaaatgtacacatactcagatttccatttatccaaaagcacttattttattttattttgctgcaaaagattcaacatttggccataggaaagaattaggttggtaggatgtcatagctgtaatatctgaagaaattgattttaggagacagccaataataatcataaagtagcaggtgttccagaaaatcagcccagaagaataaaaaagaactgtgcagaaactaaaaatttcatatattcccagaaaatgctactttaatgtctctttttggactttgttttggtactttttttttttttttttcctttctgaaaagcaaaatgttgtctgcttttggtaaccagtacaataaactgtaatggtctgtaaataaataaatattgatgtatgctatttatgtaaataggcattctgggagggaagatggctcagggtttctgtgtatcaccctgggaagtggatggattctttccagctctccggtttgcatcaggttcagttcctgctttaagaggcacaaagcctgttggatggcagccccatgccactgtgggctgggtgattccagctcagaagcagatgctcagatagccaaaccactctctcgttggtgccaacactggcatcctggtcaaagtcttagccagggtggactgaaccaccttcccatctgtcatgcaaatgtccccaagcagtgttgaaggacatgccaggtcagtgttggaacctgccctgccaggtcctgttttgtagattaaaaaaaaaatggcttcaaaaaaa

SEQ ID NO: 8 - protein, 208 aa, Mus musculus
MAPLGEVGSYFGVQDAVPFGNVPVLPVDSPVLLSDHLGQSEAGGLPRGPAVTDLDHLKGILRRRQLYCRTGFHLEIFPNGTIQGTRKDHSRFGILEFISIAVGLVSIRGVDSGLYLGMNEKGELYGSEKLTQECVFREQFEENWYNTYSSNLYKHVDTGRRYYVALNKDGTPREGTRTKRHQKFTHFLPRPVDPDKVPELYKDILSQS

SEQ ID NO: 9 - DNA, 4162 bp, Homo sapiens
agaagtccattcggctcacacatttgccccaagacaaaccacgttaaaataacacccagggtagctgctgccaccgtcttctgtctctacctccctcctggctggccaatggctctgtgttcctgggcctgctgctggctgtccagagtaggggttgcttagagctgtgtgcatccctgcgggtggtgtgggagtgggcggttgtctaaaggcaggtcccctctactgataaacaaggaccggagatagacctagaggctgacattcttggctcccccagcctacaccccccccacctcgatttcccacagagccctagggacgggtagccagctctgtggcatggtatctggaggcaggccagcaacctgatgtgcatgccacggcccgtccctctccccactcagagctgcagtagcctggaggttcagagagccgggctactctgagaagaagacaccaagtggattctgcttcccctgggacagcactgagcgagtgtggagagaggtacagccctcggcctacaagctctttagtcttgaaagcgccacaagcagcagctgctgagccatggctgaaggggaaatcaccaccttcacagccctgaccgagaagtttaatctgcctccagggaattacaagaagcccaaactcctctactgtagcaacgggggccacttcctgaggatccttccggatggcacagtggatgggacaagggacaggagcgaccagcacattcagctgcagctcagtgcggaaagcgtgggggaggtgtatataaagagtaccgagactggccagtacttggccatggacaccgacgggcttttatacggctcacagacaccaaatgaggaatgtttgttcctggaaaggctggaggagaaccattacaacacctatatatccaagaagcatgcagagaagaattggtttgttggcctcaagaagaatgggagctgcaaacgcggtcctcggactcactatggccagaaagcaatcttgtttctccccctgccagtctcttctgattaaagagatctgttctgggtgttgaccactccagagaagtttcgaggggtcctcacctggttgacccaaaaatgttcccttgaccattggctgcgctaacccccagcccacagagcctgaatttgtaagcaacttgcttctaaatgcccagttcacttctttgcagagccttttacccctgcacagtttagaacagagggaccaaattgcttctaggagtcaactggctggccagtctgggtctgggtttggatctccaattgcctcttgcaggctgagtccctccatgcaaaagtggggctaaatgaagtgtgttaaggggtcggctaagtgggacattagtaactgcacactatttccctctactgagtaaaccctatctgtgattcccccaaacatctggcatggctcccttttgtccttcctgtgccctgcaaatattagcaaagaagcttcatgccaggttaggaaggcagcattccatgaccagaaacagggacaaagaaatccccccttcagaacagaggcatttaaaatggaaaagagagattggattttggtgggtaacttagaaggatggcatctccatgtagaataaatgaagaaagggaggcccagccgcaggaaggcagaataaatccttgggagtcattaccacgccttgaccttcccaaggttactcagcagcagagagccctgggtgacttcaggtggagagcactagaagtggtttcctgataacaagcaaggatatcagagctgggaaattcatgtggatctggggactgagtgtgggagtgcagagaaagaaagggaaactggctgaggggataccataaaaagaggatgatttcagaaggagaaggaaaaagaaagtaatgccacacattgtgcttggcccctggtaagcagaggctttggggtcctagcccagtgcttctccaacactgaagtgcttgcagatcatctggggacctggtttgaatggagattctgattcagtgggttgggggcagagtttctgcagttccatcaggtcccccccaggtgcaggtgctgacaatactgctgccttacccgccatacattaaggagcagggtcctggtcctaaagagttattcaaatgaaggtggttcgacgccccgaacctcacctgacctcaactaacccttaaaaatgcacacctcatgagtctacctgagcattcaggcagcactgacaatagttatgcctgtactaaggagcatgattttaagaggctttggcccaatgcctataaaatgcccatttcgaagatatacaaaaacatacttcaaaaatgttaaacccttaccaacagcttttcccaggagaccatttgtattaccattacttgtataaatacacttcctgcttaaacttgacccaggtggctagcaaattagaaacaccattcatctctaacatatgatactgatgccatgtaaaggcctttaataagtcattgaaatttactgtgagactgtatgttttaattgcatttaaaaatatatagcttgaaagcagttaaactgattagtattcaggcactgagaatgatagtaataggatacaatgtataagctactcacttatctgatacttatttacctataaaatgagatttttgttttccactgtgctattacaaattttcttttgaaagtaggaactcttaagcaatggtaattgtgaataaaaattgatgagagtgttagctcctgtttcatatgaaattgaagtaattgttaactaaaaacaattccttagtaactgaactgtcatatttagaatggaaggaaaatgacagtttgtgaaagttcaaagcaatagtgcaattgaagaattgacctaagtaagctgacattatggttaataatagtattttagatttgtgcagcaaaataatttcataacttttttgtttttgttacttggataagatcaatctgttttattttagtaaatctttgcaggcaagttagagaaaatgcagtgtggcttaacgtctctttagtatgaagatttggccagaaaaagatacccagagaggaaatctaagataattataatggtccatactttttattgtatgaatcaaactcaagcataacattggccaaggaaaattaaataccattgctaacttgtgaaatggaagtctgtgatttcggagatgcaaagcattgtagtaaaaacaccaatgtgacctcgaccatctcagcccagatatcattcatatatctgttcaatgactattaaggtgcctactgtgtgctaggcactgtactggatactggggaccttgtctgtctggtttgctgctgtatcttctcccagggcattatatttatgatgaaagatgctgtggattcaattctttcagtcaagaataaacacagactttgtaggttcctgctgaataaagcaaatcccagaaacccagattttggaagaatcagcaaccccagcataaaataaacccctatcaaaatgtcagaggacatggcaaggtaaacttagcattttcaactttagaaccgggtcagcttcagggggactgctttcaaatcagccaaagagcctgtcagatcttcttagaaggaagaggttggtagttccctgctctgttttgaacatgctctagtttattaacctggggacattcccattgctgtcttaagtaagtctcatagccagctcctgtcacgtgactctcatatggattcattttcgggccagctctgaacaaagcatcatgaacatatgtgcttttggtcgtttgcaatgtgatggtggtggaggtaggtattggtttccttggaaggcatgataagaaagattcacaatggccaacagtgtgtatgaacaaaaaactgattggagcatcagctagtactgaaggtccttgctttgtgtcagaggcaaaggaacccaaggcgccaagtcctcagccttgagtgtactgctgacaactaaactcacaggctgcaaagcagacctctgatgaagatgcctgttatttcacatcactgtctttttgtgtatcatagtctgcaccttacaaatattaataaatgttccaataataggtgaaaaaaaaaa

SEQ ID NO: 10 - protein, 155 aa, Homo sapiens
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 11 - DNA, 3909 bp, Mus musculus
agagctgcagaaatcctgaggctcagagagccggctggagaggcagcttcagtccaggcaccctgtgacagcgcaaaggctgcccagcggacttcattcccgtcttgtgataaagtggagtgaagagagccccccagcctgccagttcttcagtgctgagcctaccaccgctgcttgctgccgagccatggctgaaggggagatcacaaccttcgcagccctgaccgagaggttcaacctgcctctaggaaactacaaaaagcccaaactgctctactgcagcaacgggggccacttcttgaggatccttcctgatggcaccgtggatgggacaagggacaggagcgaccagcacattcagctgcagctcagtgcggaaagtgcgggcgaagtgtatataaagggtacggagaccggccagtacttggccatggacaccgaagggcttttatacggctcgcagacaccaaatgaggaatgtctgttcctggaaaggctggaagaaaaccattataacacttacacctccaagaagcatgcggagaagaactggtttgtgggcctcaagaagaacgggagctgtaagcgcggtcctcggactcactatggccagaaagccatcttgtttctgcccctcccggtgtcttctgactagaggagtctgttctgagtgttctctattttggttgaccctaccatgttcccttgaccattggctgcgctaaccctcaggccacagagcctgaatttgtaagcagtgcttctaaaatagccagttcacttttctgctgagcccttccccaccaccccagcacaatttggaacacagggaccaaattgcttcaagaggacaactggctggccagtctaggtctgggtttggatatccggatgcctcagggcacctgctacaagctggacctggtgatgcaaagttgggggccatacgaagcattcaaaggggtctaagaagtgggttcttagaaagatacccactgcatgctctttccccttctgagcaatccctggttttaagcaccccaggcaattagcatgactacccttccccctaccagaaccgcacaaatatcagtaaagcaaccggtagtgaggctgaaggaatttcatggtagagagaaccccccccccccagcccccacatatggacaagacttaaaaccaaaagggagtgtggcgtttccgggacagcctagaagcatgggatctcggaacagagtaagaaggcaagagtcagccacagggagtcaggatgagcccacagcccagcagttatcaccaagttaccaaggaaacgtccacagtcagggaaagagccccggaaatggtttcttagtaatggcaagcacagagatctggggtctgaagagtgggcgtaggagcaaggagacaggatgtcttcagccgtggatgttagcctctaaaaaggaagatgttagacgggaggaggaggagagaaaaagtaatatcacgtcactgtgtgcttggccaccacgaaactggggttgtgggggtcccagtacgatggccccaaatagcccatacattcagatcatctgtattcagattcatttgtatgactgtgaaacctgatttggtggggctggtgtgggggaggtctgtgtcttgaaaggcccgcaagtcccatagatgctgctggcttccccaccatgctttcaagagcagccccagagaatgctgttcaaatgaaggttgctcagacttcaccctcttttacctccagtgcccgggttttcctgagtatccatccaggcagtgtccatagttagtgaagcatggtttcaaggcaccttggcccagtgcctataaaatgcccactcagaaatcactgaaaccgtacttcaaaaaaaaatgttgaacctcaccaaaagctttctcccaagagaccatttctgttaccattaattcttcaaactggcttcctccttaaagtaaacctggcccaagtgcccagcaaattagaaacactattcatctctgacataggacacaaacgccatgagaaggcctttcatacgccaatttaatatactgtgagacttatcttctcgttgcttcttatatagcttgaaggcattaaatagattcacaatcagccacagaggataataacagcctagaatgtaaaagctacttagttatctaacatgctacattcattcatgaggcctttcctctttgtcttccactgtgctttaagaatgggtatctaggggctggtgagatgggacaggagttcagttcctagcattcacagtgtgatgctcagacccaattctaactccaactccaggggtactgcatacacatgtacaaacccacactttttcagaaatataaaccaataatatggttgtggattaaaactgatgactgtgttcatttgtttgacagggatctaaagtcattatttgctggaaaaaaatccttatagcaacactgtcatatcttagcacagaagtcagatgacactcttgagagttcagagtgctaatgggggaactaaaccgagctatttcaatacatgattaataataatgttctttcagatgtggggatgaacacctttaatcccagcactcaggaggcagaggcaggtagatctatgtaagactgaggctaacctggtctccacagtgagttcctgggcagccaggggtatatagaaaggccccgtatcttttttttttttaatcataataatgttttgcttttttacaaaaagatgattcatggctttttaaagtctataacttaaatatgtttctgttttgtttcagtaaatcgtaggcacactggagataatacagtatggtttcccatgtctttatataaagatttggcccagaaaagtgtacagagatgaaatctaggatatttagaatagtgggtagttttttatcaaatgaacccaaacacaagtataaccctgatcaagacaaattaaaaaccactgcacaaggatgaaatgaagtctgagaccccagagactcaaagcactgatgatggtcattgtagccttaaccgtcaacctagtcaccatccgggtatttgctcaacatatactgcagacccaatgtgtgtcaggcactggatatggcccctggggacaatgtctgtcagctgtcctctctctgggcatatatttgtcaagagagatgctatggattcatttccttcagtcacgtcaaaacatggacttcatagtttcctactgaatcaacagtatgttctagaaaccctcaacggtcagtcacaccaccccacccccaacaaagtgacagagacaggctttgccaagtaaacccagggctttcaatgttagtcctctcagaggagtggagtagtttcaaatcaggaaggaaaaaacaaacaaaaaagctgtcggatcttgcagaaagggagcagagagactgtccctgtgctggtgggagcactgatcataggaacattcggttgtgtacgaagtcccaagaccatctgcttgtcacatgcctcctgtatgcactcaattccaagctgactgcaacctggtgtctaacactcactacgaactgtgaggagcatatgggcttttgacaattagtgacacagtagatatggagctgggtgtatcccctgatggccagcagtgtgcccctacagaaatcctaactggtacatcagctgccattgaagacgaggcccctgctttgtgttgaaggcagtggagcccgagtcctcagctctgcagagaacaccgcctaaaagcaatcacggtcgctggctgcaaagcaggctctgatggagacgacgcctgacattcactctgcacccatactgtattacactctgcaccttatcaatattaataaatgttgtattaatggc

SEQ ID NO: 12 - protein, 155 aa, Mus musculus
MAEGEITTFAALTERFNLPLGNYKKPKLLYCSNGGHFLRILPDGTVDGTRDRSDQHIQLQLSAESAGEVYIKGTETGQYLAMDTEGLLYGSQTPNEECLFLERLEENHYNTYTSKKHAEKNWFVGLKKNGSCKRGPRTHYGQKAILFLPLPVSSD

SEQ ID NO: 13 - DNA, 6774 bp, Homo sapiens
cggccccagaaaacccgagcgagtagggggcggcgcgcaggagggaggagaactgggggcgcgggaggctggtgggtgtggggggtggagatgtagaagatgtgacgccgcggcccggcgggtgccagattagcggacgcggtgcccgcggttgcaacgggatcccgggcgctgcagcttgggaggcggctctccccaggcggcgtccgcggagacacccatccgtgaaccccaggtcccgggccgccggctcgccgcgcaccaggggccggcggacagaagagcggccgagcggctcgaggctgggggaccgcgggcgcggccgcgcgctgccgggcgggaggctggggggccggggccggggccgtgccccggagcgggtcggaggccggggccggggccgggggacggcggctccccgcgcggctccagcggctcggggatcccggccgggccccgcagggaccatggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctgattttaatggccacatctaatctcatttcacatgaaagaagaagtatattttagaaatttgttaatgagagtaaaagaaaataaatgtgtatagctcagtttggataattggtcaaacaattttttatccagtagtaaaatatgtaaccattgtcccagtaaagaaaaataacaaaagttgtaaaatgtatattctcccttttatattgcatctgctgttacccagtgaagcttacctagagcaatgatctttttcacgcatttgctttattcgaaaagaggcttttaaaatgtgcatgtttagaaacaaaatttcttcatggaaatcatatacattagaaaatcacagtcagatgtttaatcaatccaaaatgtccactatttcttatgtcattcgttagtctacatgtttctaaacatataaatgtgaatttaatcaattcctttcatagttttataattctctggcagttccttatgatagagtttataaaacagtcctgtgtaaactgctggaagttcttccacagtcaggtcaattttgtcaaacccttctctgtacccatacagcagcagcctagcaactctgctggtgatgggagttgtattttcagtcttcgccaggtcattgagatccatccactcacatcttaagcattcttcctggcaaaaatttatggtgaatgaatatggctttaggcggcagatgatatacatatctgacttcccaaaagctccaggatttgtgtgctgttgccgaatactcaggacggacctgaattctgattttataccagtctcttcaaaaacttctcgaaccgctgtgtctcctacgtaaaaaaagagatgtacaaatcaataataattacacttttagaaactgtatcatcaaagattttcagttaaagtagcattatgtaaaggctcaaaacattaccctaacaaagtaaagttttcaatacaaattctttgccttgtggatatcaagaaatcccaaaatattttcttaccactgtaaattcaagaagcttttgaaatgctgaatatttctttggctgctacttggaggcttatctacctgtacatttttggggtcagctctttttaacttcttgctgctctttttcccaaaaggtaaaaatatagattgaaaagttaaaacattttgcatggctgcagttcctttgtttcttgagataagattccaaagaacttagattcatttcttcaacaccgaaatgctggaggtgtttgatcagttttcaagaaacttggaatataaataattttataattcaacaaaggttttcacattttataaggttgatttttcaattaaatgcaaatttgtgtggcaggatttttattgccattaacatatttttgtggctgctttttctacacatccagatggtccctctaactgggctttctctaattttgtgatgttctgtcattgtctcccaaagtatttaggagaagccctttaaaaagctgccttcctctaccactttgctggaaagcttcacaattgtcacagacaaagatttttgttccaatactcgttttgcctctatttttcttgtttgtcaaatagtaaatgatatttgcccttgcagtaattctactggtgaaaaacatgcaaagaagaggaagtcacagaaacatgtctcaattcccatgtgctgtgactgtagactgtcttaccatagactgtcttacccatcccctggatatgctcttgttttttccctctaatagctatggaaagatgcatagaaagagtataatgttttaaaacataaggcattcgtctgccatttttcaattacatgctgacttcccttacaattgagatttgcccataggttaaacatggttagaaacaactgaaagcataaaagaaaaatctaggccgggtgcagtggctcatgcctatattccctgcactttgggaggccaaagcaggaggatcgcttgagcccaggagttcaagaccaacctggtgaaaccccgtctctacaaaaaaacacaaaaaatagccaggcatggtggcgtgtacatgtggtctcagatacttgggaggctgaggtgggagggttgatcacttgaggctgagaggtcaaggttgcagtgagccataatcgtgccactgcagtccagcctaggcaacagagtgagactttgtctcaaaaaaagagaaattttccttaataagaaaagtaatttttactctgatgtgcaatacatttgttattaaatttattatttaagatggtagcactagtcttaaattgtataaaatatcccctaacatgtttaaatgtccatttttattcattatgctttgaaaaataattatggggaaatacatgtttgttattaaatttattattaaagatagtagcactagtcttaaatttgatataacatctcctaacttgtttaaatgtccatttttattctttatgtttgaaaataaattatggggatcctatttagctcttagtaccactaatcaaaagttcggcatgtagctcatgatctatgctgtttctatgtcgtggaagcaccggatgggggtagtgagcaaatctgccctgctcagcagtcaccatagcagctgactgaaaatcagcactgcctgagtagttttgatcagtttaacttgaatcactaactgactgaaaattgaatgggcaaataagtgcttttgtctccagagtatgcgggagacccttccacctcaagatggatatttcttccccaaggatttcaagatgaattgaaatttttaatcaagatagtgtgctttattctgttgtattttttattattttaatatactgtaagccaaactgaaataacatttgctgttttataggtttgaagaacataggaaaaactaagaggttttgtttttatttttgctgatgaagagatatgtttaaatatgttgtattgttttgtttagttacaggacaataatgaaatggagtttatatttgttatttctattttgttatatttaataatagaattagattgaaataaaatataatgggaaataatctgcagaatgtgggttttcctggtgtttccctctgactctagtgcactgatgatctctgataaggctcagctgctttatagttctctggctaatgcagcagatactcttcctgccagtggtaatacgattttttaagaaggcagtttgtcaattttaatcttgtggatacctttatactcttagggtattattttatacaaaagccttgaggattgcattctattttctatatgaccctcttgatatttaaaaaacactatggataacaattcttcatttacctagtattatgaaagaatgaaggagttcaaacaaatgtgtttcccagttaactagggtttactgtttgagccaatataaatgtttaactgtttgtgatggcagtattcctaaagtacattgcatgttttcctaaatacagagtttaaataatttcagtaattcttagatgattcagcttcatcattaagaatatcttttgttttatgttgagttagaaatgccttcatatagacatagtctttcagacctctactgtcagttttcatttctagctgctttcagggttttatgaattttcaggcaaagctttaatttatactaagcttaggaagtatggctaatgccaacggcagtttttttcttcttaattccacatgactgaggcatatatgatctctgggtaggtgagttgttgtgacaaccacaagcactttttttttttttaaagaaaaaaaggtagtgaatttttaatcatctggactttaagaaggattctggagtatacttaggcctgaaattatatatatttggcttggaaatgtgtttttcttcaattacatctacaagtaagtacagctgaaattcagaggacccataagagttcacatgaaaaaaatcaatttatttgaaaaggcaagatgcaggagagaggaagccttgcaaacctgcagactgctttttgcccaatatagattgggtaaggctgcaaaacataagcttaattagctcacatgctctgctctcacgtggcaccagtggatagtgtgagagaattaggctgtagaacaaatggccttctctttcagcattcacaccactacaaaatcatcttttatatcaacagaagaataagcataaactaagcaaaaggtcaataagtacctgaaaccaagattggctagagatatatcttaatgcaatccattttctgatggattgttacgagttggctatataatgtatgtatggtattttgatttgtgtaaaagttttaaaaatcaagctttaagtacatggacatttttaaataaaatatttaaagacaatttagaaaattgccttaatatcattgttggctaaatagaataggggacatgcatattaaggaaaaggtcatggagaaataatattggtatcaaacaaatacattgatttgtcatgatacacattgaatttgatccaatagtttaaggaataggtaggaaaatttggtttctatttttcgatttcctgtaaatcagtgacataaataattcttagcttattttatatttccttgtcttaaatactgagctcagtaagttgtgttaggggattatttctcagttgagactttcttatatgacattttactatgttttgacttcctgactattaaaaataaatagtagatacaattttcataaagtgaagaattatataatcactgctttataactgactttattatatttatttcaaagttcatttaaaggctactattcatcctctgtgatggaatggtcaggaatttgttttctcatagtttaattccaacaacaatattagtcgtatccaaaataacctttaatgctaaactttactgatgtatatccaaagcttctcattttcagacagattaatccagaagcagtcataaacagaagaataggtggtatgttcctaatgatattatttctactaatggaataaactgtaatattagaaattatgctgctaattatatcagctctgaggtaatttctgaaatgttcagactcagtcggaacaaattggaaaatttaaatttttattcttagctataaagcaagaaagtaaacacattaatttcctcaacatttttaagccaattaaaaatataaaagatacacaccaatatcttcttcaggctctgacaggcctcctggaaacttccacatatttttcaactgcagtataaagtcagaaaataaagttaacataactttcactaacacacacatatgtagatttcacaaaatccacctataattggtcaaagtggttgagaatatattttttagtaattgcatgcaaaatttttctagcttccatcctttctccctcgtttcttctttttttgggggagctggtaactgatgaaatcttttcccaccttttctcttcaggaaatataagtggttttgtttggttaacgtgatacattctgtatgaatgaaacattggagggaaacatctactgaatttctgtaatttaaaatattttgctgctagttaactatgaacagatagaagaatcttacagatgctgctataaataagtagaaaatataaatttcatcactaaaatatgctattttaaaatctatttcctatattgtatttctaatcagatgtattactcttattatttctattgtatgtgttaatgattttatgtaaaaatgtaattgcttttcatgagtagtatgaataaaattgattagtttgtgttttcttgtctccc

SEQ ID NO: 14 - protein, 288 aa, Homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 15 - DNA, 695 bp, Mus musculus
ggccccgggccgttgtacactcaaggggctctctcggcttcaggaagagtccggctgcactgggctgggagcccggcgggacacggactgggaggctggcagcccgcgggcgagccgcgctggggggccgaggccggggtcggggccggggagccccaagagctgccacagcggggtcccggggccgcggaagggccatggctgccagcggcatcacctcgcttcccgcactgccggaggacggcggcgccgccttcccaccaggccacttcaaggaccccaagcggctctactgcaagaacggcggcttcttcctgcgcatccatcccgacggccgcgtggatggcgtccgcgagaagagcgacccacacgtcaaactacaactccaagcagaagagagaggagttgtgtctatcaagggagtgtgtgccaaccggtaccttgctatgaaggaagatggacggctgctggcttctaagtgtgttacagaagagtgtttcttctttgaacgactggaatctaataactacaatacttaccggtcacggaaatactccagttggtatgtggcactgaaacgaactgggcagtataaactcggatccaaaacgggacctggacagaaggccatactgtttcttccaatgtctgctaagagctgactcacttttgacactgtcactgagacactgtca

SEQ ID NO: 16 - protein, 154 aa, Mus musculus
MAASGITSLPALPEDGGAAFPPGHFKDPKRLYCKNGGFFLRIHPDGRVDGVREKSDPHVKLQLQAEERGVVSIKGVCANRYLAMKEDGRLLASKCVTEECFFFERLESNNYNTYRSRKYSSWYVALKRTGQYKLGSKTGPGQKAILFLPMSAKS

SEQ ID NO: 17 - DNA, 1220 bp, Homo sapiens
gggagcgggcgagtaggagggggcgccgggctatatatatagcggctcggcctcgggcgggcctggcgctcagggaggcgcgcactgctcctcagagtcccagctccagccgcgcgctttccgcccggctcgccgctccatgcagccggggtagagcccggcgcccgggggccccgtcgcttgcctcccgcacctcctcggttgcgcactcctgcccgaggtcggccgtgcgctcccgcgggacgccacaggcgcagctctgccccccagcttcccgggcgcactgaccgcctgaccgacgcacggccctcgggccgggatgtcggggcccgggacggccgcggtagcgctgctcccggcggtcctgctggccttgctggcgccctgggcgggccgagggggcgccgccgcacccactgcacccaacggcacgctggaggccgagctggagcgccgctgggagagcctggtggcgctctcgttggcgcgcctgccggtggcagcgcagcccaaggaggcggccgtccagagcggcgccggcgactacctgctgggcatcaagcggctgcggcggctctactgcaacgtgggcatcggcttccacctccaggcgctccccgacggccgcatcggcggcgcgcacgcggacacccgcgacagcctgctggagctctcgcccgtggagcggggcgtggtgagcatcttcggcgtggccagccggttcttcgtggccatgagcagcaagggcaagctctatggctcgcccttcttcaccgatgagtgcacgttcaaggagattctccttcccaacaactacaacgcctacgagtcctacaagtaccccggcatgttcatcgccctgagcaagaatgggaagaccaagaaggggaaccgagtgtcgcccaccatgaaggtcacccacttcctccccaggctgtgaccctccagaggacccttgcctcagcctcgggaagcccctgggagggcagtgccgagggtcaccttggtgcactttcttcggatgaagagtttaatgcaagagtaggtgtaagatatttaaattaattatttaaatgtgtatatattgccaccaaattatttatagttctgcgggtgtgttttttaattttctggggggaaaaaaagacaaaacaaaaaaccaactctgacttttctggtgcaacagtggagaatcttaccattggatttctttaacttgt

SEQ ID NO: 18 - protein, 206 aa, Homo sapiens
MSGPGTAAVALLPAVLLALLAPWAGRGGAAAPTAPNGTLEAELERRWESLVALSLARLPVAAQPKEAAVQSGAGDYLLGIKRLRRLYCNVGIGFHLQALPDGRIGGAHADTRDSLLELSPVERGVVSIFGVASRFFVAMSSKGKLYGSPFFTDECTFKEILLPNNYNAYESYKYPGMFIALSKNGKTKKGNRVSPTMKVTHFLPRL

SEQ ID NO: 19 - DNA, 3030 bp, Mus musculus
gcgcggggactatcccgccaccgttgcgtccctatttgctctcgctacttaggtctgtgcgcagcactcaccgaactcacggcccgcagctcgaactcacgcacggcccgcgggccgggatggcgaaacgcgggccgaccacagggacgctgctgcccagggtcctgctggccctggtggtggccctggcggaccgagggaccgccgcacccaacggcacgcggcacgcagaattggggcacggctgggacggcttggtggcccgctcgctggcacgcctgccggtggccgcgcagcccccgcaggcggcggtccgcagcggcgcaggggactacctgctgggcctcaaaaggcttcggcggctctactgcaacgtgggcatcggattccacctgcaggtgctgcccgacggccgcatcggtggtgtgcacgcagacacgagggacagtcttctggagctctctccggtgcagcgaggcgtggtgagcatcttcggagtggccagccggttcttcgtggccatgagcagcaggggcaagctcttcggtgtgcctttctttaccgacgagtgtaaattcaaagaaatacttctgcccaacaactacaacgcctacgaatcctacgcgtaccccggtatgttcatggccctcagtaagaacgggcggaccaagaaggggaaccgagtgtcgcctaccatgaaggtaacccacttccttcctagactgtgaccctccggagccctgcctcagcctcggaagcacaccctacccctcaggaggagcactttctctcgatggagaattgtttgcaaaaaaactggtctaagatatttaaattatttaaatatgtatatatggacgcccaattatttataattttatgtattctcattttcggggggggggatatgaccaaaagaacaaacaaattgcagctcagacctctttggaatagcggaacagacttctttcttcactatcaaagatatgggtgtgatgctgtttcatgtgtgcctctaaaacttggtgacatcagtttccaagggtgcctggcctgtaactggaaaggcctgcctgggacctctgaggcagtgagaaggaccctgagcgttcccgatcgggagcattctgccgtcgccgctccctctgcttcctttggtatgaaccctgtcggatcggtttactccagggacagaagtcctcccgcctctgtttttagatctccaagactgatctttgaactctcttgcagtcaatcatcttcttggacctaccggataggagacccttagacaactttataaactcctgtttgccatttttggactggccaacagggcacgttgcttgtagccactggaactttgcttttctggagaggaactaggaatggacaagagggtgtgtgcgctcgccacaactcaaaactctggggatgaaattctgttttgtgatagaggatgatttgttgggatataacaatgtattttgcaagaatcaaactgagaaaaacaggcttccctgaatatgggaagctttgtgctggaactccataatttaagggtgagctgccctttcccaccccaggcagcctttatgcaggcagactgcaggagtccgaattccggtgctgccccaaggtgaaggaattctatccccatctacctcagagttcctgagaccctggtgactaagctggaggctctgctgggtgcatctgcctgcttctcttccacatgtagtatctggacccttgtcacgttccagctatctttacccaatgatttacttttagtaaggggtattcatggtgaaaatatgcacgaccagctgtcggagtgcactatatggtcaccaagtcaagtaacccttatatttttctttatatattttacgaatgtaacccctgtcactgagaccaaaatggaagagcagggtcggtggcatttaaaaaaaagggctgaggtgagggagaacaattaattttaatgaatgactttggagtttatacaaaatgaccttagctcgcttcagggaatgcttctccccaagatgttaagactctgctgggagacttctgagcaacctcccgaattaactttatgggaggctacagacagcaagactggaaaatctcattggcattttttttttttgtctttcacattcctttagaaaactctttgtttggatgctaatgggatacttaaaatactattctgtaccacagcccaagatggaagaagccacaccccaaagctgaggtgggagctcctcccaaacttcctttctgtctggtggctcacaggacaataagattttgtgttttttaaatccaggccctaggctccaagagtgttggggagaagacagttgaaatgtcttttcttcttgttattttctaaaagacggtctcatagcccaggctgtacctgaacttgctatgtcactaaagatgaccttgaatttctgatcctcttgccccatttcctagtgctaaggttacaggcattgcccaccacgcccactccttaggtgctgggaattgaaatcaagtgcctgccaggccactccacagagataggcctccctcttttttgtgggggaggaaatgtggggtcttactctgtagccaaggctggcctactgcttgagcccaagtgttgagactacaggtagaaggcacctgccctgttctgatgggattctctgcagctatgttgcttcaggcaggctgtggtcctacctgacacctattacctgtttcttcagccagctagctccctttcctgcgcccagggacaagactgtatctttggaattttgttaccagaatgggtttgatgtttctgctctgtatctgccactgttaccgagtcggtctgtagttactacaggtgtcgacccagagatagtatgtgctatgcacactggatgctccatccaaagagaagcattcaatcatgtatagacagccccatggactgatgggaatgattgggagagatattaaaatgacaaacgtgtctgc

SEQ ID NO: 20 - protein, 202 aa, Mus musculus
MAKRGPTTGTLLPRVLLALVVALADRGTAAPNGTRHAELGHGWDGLVARSLARLPVAAQPPQAAVRSGAGDYLLGLKRLRRLYCNVGIGFHLQVLPDGRIGGVHADTRDSLLELSPVQRGVVSIFGVASRFFVAMSSRGKLFGVPFFTDECKFKEILLPNNYNAYESYAYPGMFMALSKNGRTKKGNRVSPTMKVTHFLPRL

SEQ ID NO: 21 - DNA, 5399 bp, Homo sapiens
ggggaagcttcgcaggcgtgcacggagcagtgagatcactggcgttataaatatcccggtgccagcgcggagatccgctcgggtggcctctctcttcccctctccccttctcttccccgaggctatgtccacccggtgcggcgaggcgggcagagccagaggcacgcagccgcacaggggctacagagcccagaatcagccctacaagatgcacttaggacccccgcggctggaagaatgagcttgtccttcctcctcctcctcttcttcagccacctgatcctcagcgcctgggctcacggggagaagcgtctcgcccccaaagggcaacccggacccgctgccactgataggaaccctagaggctccagcagcagacagagcagcagtagcgctatgtcttcctcttctgcctcctcctcccccgcagcttctctgggcagccaaggaagtggcttggagcagagcagtttccagtggagcccctcggggcgccggaccggcagcctctactgcagagtgggcatcggtttccatctgcagatctacccggatggcaaagtcaatggatcccacgaagccaatatgttaagtgttttggaaatatttgctgtgtctcaggggattgtaggaatacgaggagttttcagcaacaaatttttagcgatgtcaaaaaaaggaaaactccatgcaagtgccaagttcacagatgactgcaagttcagggagcgttttcaagaaaatagctataatacctatgcctcagcaatacatagaactgaaaaaacagggcgggagtggtatgtggccctgaataaaagaggaaaagccaaacgagggtgcagcccccgggttaaaccccagcatatctctacccattttctgccaagattcaagcagtcggagcagccagaactttctttcacggttactgttcctgaaaagaaaaagccacctagccctatcaagccaaagattcccctttctgcacctcggaaaaataccaactcagtgaaatacagactcaagtttcgctttggataatattcctcttggccttgtgagaaaccattctttcccctcaggagtttctataggtgtcttcagagttctgaagaaaaattactggacacagcttcagctatacttacactgtattgaagtcacgtcatttgtttcaatgtgactgaaacaaaatgttttttgataggaaggaaactggaattctttgtactaatacagggagcacactccttcagttcagcaagacataaagccttttgctttatgcttgagggatatttagaactttgtattttcggaaagttaaataacagggactacgtatttttctgacttttacagattaacctgaaagaacatacatgatacatttttatttttggtttccaaagaatattttgatgcagataaaatattttgttaacttttgtttttttttgtttgttttcttaaaagtacctctgcattgagcatattttcttacttttattattttaattaatatgacataagcaatcattttatgctgtttatgaattataaatgtgtttatagctcatttgtaatatggaaatcttttacatttttcctattcactgcacttttttattgtttttatttctagccatacctcagataatatgtttagttttacattttaaaatgtttaaattctctttcacagcaccaaaggctcagcttggatttgtgtgtatgtgtatgtcaattcatgacattatgtggaatcctaaacctttggtggctgggatatgatgggttagaagcaaggagaaaatataaggactttttgatggaattaaatgtgggaggtaaggaaaaggatttagaggtaaaagtacactaagtttgcaacatttattgagatctaagtctgtcttgccttcatttctctttttatctcccccttgccctcattcttgaacagctggaggaatacattttattctgtccatgaagcatacactatgaaattcaagtgcttaaaaatacttctatgactctctgctatcccactgtatagatccacagggagcaaacacttagaaatgatagagaactgaaggagatcaatggtttaacagttatccatgccaagtcccattgtcagaaatattcttattactcagtcaaacactctttgagcttcccttcctaaaggtaaccaatccagtgaatagatgtgcccttttataaggaaacttctgatgtttattaaaaaaactggccttttgatagaggtaacttaatttgggaatttgttgtgttgaaatggcatttaatttcaacctaaatactgactgctggacataaatcacagaaaatttaacttaagaaaatttacaaaatttattctcaggtaatcattttaataaagttctgcaaaatacacgtttatcttacattcagaaatgtggcaaaaaaggcatagctaaaggctaaacatatggctttagtagtaacaaaagggttcatagaaacttcatggtttgcatttaaacatgtttaaagtgtacttataaactatttttttcttaaagcaaactatgatttattttggtgcacaaatacaaagtggaaacttaccaaaattgaactagctaccatataagcagattgctttaatttgatgggaaaatagtacacacatatatataacaaataatatattaaaaaacccatccatcaactaaaacattatatgtatacatcagtatagtgttttattataaagccaattatctgattaagcattctttccactgaatgcataatgtttaaatagcataaaatgaaatgctacaaaaattgaactaatttatactttaaagtatttctgggttaaatgaaacaatgaaattttttagtatgttcaactctcatccaaatggcatatgaccctgtttacacagcctaaagctaaaaatattactctagtttattctaatctattgttaagtattgtgcactgtataccaagttcttagggcacatgaaaaattttagctgccaaacaggaactagtaaacatatgttcctaataagtgaagggaaagataataatgatggtcaacaataagccacgtcaatgcataagttgtataggctaaatgttgcttgtaggctacattaaactcaaatgtaatagtttatcttatactcctggtttgatttgattagcatattaacgtgaaagtaggatagctactaaatatatattatgcaagtcaggaatcattaatttcaaaatttaaagccatgctaaaattaaaaagaaaatattaaattacacaattacacttgtctttactggccatacaaaatgattttttttttttttttgagacagagtcttgctctgtcaccaggctggagtgcagtggcatgatctcggctcactgcaacctccaactccctggtttaagggattctcctgcctcagcctcccaagtagctgggattacagactcatgccaccacgccagctaatttttgtatttttagtagagacggggtttcaccatgttggtcaggatggtctcaatcctggcctcttgatagtcctgacctcatgatctgcccacctcggcctccccaaagtgctgggattacaggtacaatgatgtataattaatgcttagtgaagcataaagttacctacatcaattaattaaatgaacttatgtacagaaaacatgtataaatataagtctatactaatgcttacaactttctaagagggttcttgcttatgtagctttttattattttaagtaactagaaccaccaaatatcaaataaaattatttggttatggttatgttcatctaaacacaacaataacttttatattaatatttaggagtctattttgtctataggtgacaaacatctccagactaacatgtcagttttatcaattatattatgtttaattatttaagatttctttatgtggaacatctatagagataaatagaaattttcaataagatgtagtaacactgtgatttatctttcaagagtctctcttcacttccttctaaagagactaatttgagagtacaggtgcatattaattttcttggttctttcagctgaattatattggtccagaagttcaaaatcatgtgacaataataagggatactgacagaagttatttccaagtttgtgtatatattataaaaattacatatataaaactaaggcttttatttctgttatttttaagcttttatttcttgtagctaaaaataaaacatcataaatctggtaggtaaatttcttattaaatcaatcttgaaatagaaaatgtaataactttcttaccattaacattttttacccttccatagaagggagggaataaatcatgacttatcccattttcaataacaaaacgaaactatggcactaaccaaaaacttgcattctggcataatttttacagttgcagagaattgtttctgggctcattaaaaaaagtagtattgcagacattgctgcaatgggaagcagacaataacttcttaaaggaattctacacctcctttaagatttacttaattgctacatctaaattctgataatttaaaatccattttaggtgataaaattttttaaaagttttgaaggaaacctctggataaatggacaaggcctaatttttttttgtagtcaatccaactgtactggccaatttttgaaataagattatatgattaggtattagcagagacaaagagttacctcctccatcttactctgccctatttgaaagtctcaggggagaaaagggaacaagatgctgatccaacctgagtggagtcaggtgaggcatctttacatctaagaatttttttttaaattttattattattatacttcaagttctagggtacatgtccacaatgcacatgtctgtcacacatgcacacatgtgccatgctggtgtgctgcacccaccaacctgtcatccagcattaggtatatctcctaatgctatccctcccctctccacccaccccacagcaggccccggtatgtgatgttccccttcgtgtgtccatgtgttcttattgttcaattcccacctatgagtgagaatatgtggtgtttggtttttggtccttgcaatagtttgctgagaatgatggtttccagcttcatccatgtccctacaaagaacatgaactcatcattttttatggctgcatagtattccatggtgtatatgtgccacattttcttaatccagtctatcattgttggacatttgggttggttccaagtctttgctattgtgaatagtgctgcaataaacatatgtgtgcatgtgtctttaaaaaaaaaaa

SEQ ID NO: 22 - protein, 268 aa, Homo sapiens
MSLSFLLLLFFSHLILSAWAHGEKRLAPKGQPGPAATDRNPRGSSSRQSSSSAMSSSSASSSPAASLGSQGSGLEQSSFQWSPSGRRTGSLYCRVGIGFHLQIYPDGKVNGSHEANMLSVLEIFAVSQGIVGIRGVFSNKFLAMSKKGKLHASAKFTDDCKFRERFQENSYNTYASAIHRTEKTGREWYVALNKRGKAKRGCSPRVKPQHISTHFLPRFKQSEQPELSFTVTVPEKKKPPSPIKPKIPLSAPRKNTNSVKYRLKFRFG

SEQ ID NO: 23 - DNA, 2501 bp, Mus musculus
gcaggcgtgcacggagcagtgagatcactggcgttataaatatcccggtgccagcgccgagatccgctcgggtggcctctctctctccccctctccctctcccttccccgaggctatgtccaccctgtgcggcgagggaggcagcgccagaggcacgcagccgcgcgggggctacggagcccggagccagccctgcaagatgcacttaggacccccgcggccggaagaatgagcctgtccttgctcttcctcatcttctgcagccacctgatccacagcgcttgggctcacggggagaagcgtctcactcccgaagggcaacccgcgcctcctaggaacccgggagactccagcggcagccggggcagaagtagcgcgacgttttcttcgtcttctgcctcctcaccagtcgcagcttctccgggcagccaaggaagcggctcggaacatagcagtttccagtggagcccttcggggcgccggaccggcagcctgtactgcagagtgggcatcggtttccatctgcagatctacccggatggcaaagtcaatggctcccacgaagccagtgtgttaagtattttggaaatatttgctgtgtctcaggggattgtaggaatacgaggagttttcagcaacaaatttttagcgatgtcaaaaaaaggaaaactccatgcaagtgccaaatttacggatgactgtaagttcagggagagattccaagaaaacagctataatacctatgcgtccgcgatccacagaactgaaaagacaggccgagagtggtacgtggccctgaacaagagagggaaagccaagagaggctgcagcccacgggtcaaaccccaacacgtctccacccacttcctacccaggttcaagcagtccgagcaaccggaactttccttcaccgtcactgttccagaaaagaaaaagccaccggtgaaaccaaaggtgcccctgtcgcagcctcgcagaagtcccagcccagtgaagtacagactgaagtttcgctttggatgatgctcgtccatcctggccttgtgggaaacaactctctatacaggcgtcatcggagccctgaaggaaactcggatacagcatccctctgctgtgcctacacgtgtggaagtcaggtcattttgtttcgctttgactggaactaaactgctttctaaaatttaagtcgaaacagaagtgggattctctgtagcgatccagggagctcatctcttcagatcaccgaggatataaaggcttcgtgatttgaggatggttagcattctgcgttttcagaccagtttgatagcctggaactatgtacttttctcatttttacagatcaaccggaaagaacatacatgatacatgtttatttttgttttccaaagaatattttgatgcagataaaatattttataaacttttgtttttttaaggtacctcagcattgagcatattttcttacttttattattttaattaacgtgacacaagcagttggtttattgctgttcatggattataaacattttaatagtttgtgtggtaggatctggatctcctttgcgtttttctctctactacagcatcgtttactttattttagctatacctcaagtcgtataaatttagttctacattttaaaaaaaatttttaaatgtgctttcgaagccccaaaaggctcagcttggaggtgtgcattgtgtatgaagttatggcattatgtggaatctggccatattagtggctgggctcaatgatcagaaggaggaaaatatgaggacgttttgatggatttaaaggctttatgtaaagaaaaggacagaggccaccgcacactaagtgtgcagcgtccacagagatttaactctgtcttttgcttcttctccttttatctgccccctgccctcattcaagaccagctgaaggaataagttttattctgtccaccaagcatacactaggaaactggagtgcttaaaaatacttctataactccctgttatccttactgtatggacccacagggagtaactaatcagggtgtgtgtgtgtgtgtgtgtgtgtgtgtttgtgtgtgtgtgtgtgtgtgtgtgtgtgtgtgtgtgtgtagctgaaggagatgaatcactaaccaagttttcatgtcaagtctcatggtcaaaaatacttctactattcagtcaaataccctttgagctttctaccctaaaggtaatgactggaatgagtgcatctgctctgctctaagaaaacctggtgcaccctagaagcttctgcccttgcgacccaggagcttaattcgggaatgtgatgagctgaaaatgacacttcattctcaacccaaattccaaaccctcaacagaaaccacagacggtttagcttacaaagagttacaaaatgtattttcttggataatcctttttatggagatctatgaagtataagtgttcaccttatattcctaaatgctgcaataaagaggcgtagatgacc

SEQ ID NO: 24 - protein, 264 aa, Mus musculus
MSLSLLFLIFCSHLIHSAWAHGEKRLTPEGQPAPPRNPGDSSGSRGRSSATFSSSSASSPVAASPGSQGSGSEHSSFQWSPSGRRTGSLYCRVGIGFHLQIYPDGKVNGSHEASVLSILEIFAVSQGIVGIRGVFSNKFLAMSKKGKLHASAKFTDDCKFRERFQENSYNTYASAIHRTEKTGREWYVALNKRGKAKRGCSPRVKPQHVSTHFLPRFKQSEQPELSFTVTVPEKKKPPVKPKVPLSQPRRSPSPVKYRLKFRFG

SEQ ID NO: 25 - DNA, 856 bp, Homo sapiens
accttgcgtccgcagtaccgacccgcacgctcttcagcgcatccctagtgaaggaggttctcccccagcccgtggctgttgcacttgctggtcctctgcctccaagcccagcatgtgagggagcagagcctggtgacggatcagctcagccgccgcctcatccggacctaccaactctacagccgcaccagcgggaagcacgtgcaggtcctggccaacaagcgcatcaacgccatggcagaggacggcgaccccttcgcaaagctcatcgtggagacggacacctttggaagcagagttcgagtccgaggagccgagacgggcctctacatctgcatgaacaagaaggggaagctgatcgccaagagcaacggcaaaggcaaggactgcgtcttcacggagattgtgctggagaacaactacacagcgctgcagaatgccaagtacgagggctggtacatggccttcacccgcaagggccggccccgcaagggctccaagacgcggcagcaccagcgtgaggtccacttcatgaagcggctgccccggggccaccacaccaccgagcagagcctgcgcttcgagttcctcaactacccgcccttcacgcgcagcctgcgcggcagccagaggacttgggcccccgagccccgataggtgctgcctggccctccccacaatgccagaccgcagagaggctcatcctgtagggcacccaaaactcaagcaagatgagctgtgcgctgctctgcaggctggggaggtgctgggggagccctgggttccggttgttgatattgtttgctgttgggtttttgctgtttttttttttttttttttttttaaaacaaaagag

SEQ ID NO: 26 - protein, 140 aa, Homo sapiens
MAEDGDPFAKLIVETDTFGSRVRVRGAETGLYICMNKKGKLIAKSNGKGKDCVFTEIVLENNYTALQNAKYEGWYMAFTRKGRPRKGSKTRQHQREVHFMKRLPRGHHTTEQSLRFEFLNYPPFTRSLRGSQRTWAPEPR

SEQ ID NO: 27 - DNA, 1188 bp, Mus musculus
cggccaccggctccagcagcggctcagaggggttcggcgcgcgcggcgagcacgacattccacgagccgcgtcgggatagccgctggcctcccgcaccccgacctccctcagcctccgcaccttcggcttgtccccccgcggcctccagtgggacggcgtgaccccgctcgggctctcagtgctcccggggccgcgcgccatgggcagcccccgctccgcgctgagctgcctgctgttgcacttgctggttctctgcctccaagcccaggaaggcccgggcggggggcctgcgctgggcagggagcccacttccctgctccgagctggccgggagccccagggtgtttcccaacaggtaactgttcagtcctcacctaattttacacagcatgtgagggagcagagcctggtgacggatcagctcagccgccgcctcatccggacctaccagctctacagccgcaccagcgggaagcacgtgcaggtcctggccaacaagcgcatcaacgccatggcagaagacggagaccccttcgcgaagctcattgtggagaccgatacttttggaagcagagtccgagttcgcggcgcagagacaggtctctacatctgcatgaacaagaaggggaagctaattgccaagagcaacggcaaaggcaaggactgcgtattcacagagatcgtgctggagaacaactacacggcgctgcagaacgccaagtacgagggctggtacatggcctttacccgcaagggccggccccgcaagggctccaagacgcgccagcatcagcgcgaggtgcacttcatgaagcgcctgccgcggggccaccacaccaccgagcagagcctgcgcttcgagttcctcaactacccgcccttcacgcgcagcctgcgcggcagccagaggacttgggccccggagccccgataggcgctcgcccagctcctccccacccagccggccgaggaatccagcgggagctcggcggcacagcaaaggggaggggctggggagctgccttctagttgtgcatattgtttgctgttgggtttttttgttttttgttttttgtttttgttttttgttttttaaacaaaagagaggctctatttttgtattccaaaaaaaaaaaaaaaaaaaaaaaaaaaaaaaaaaaaaaaaaaaaaaaaaaaaaaaaaaaaaa

SEQ ID NO: 28 - protein, 244 aa, Mus musculus
MGSPRSALSCLLLHLLVLCLQAQEGPGGGPALGREPTSLLRAGREPQGVSQQVTVQSSPNFTQHVREQSLVTDQLSRRLIRTYQLYSRTSGKHVQVLANKRINAMAEDGDPFAKLIVETDTFGSRVRVRGAETGLYICMNKKGKLIAKSNGKGKDCVFTEIVLENNYTALQNAKYEGWYMAFTRKGRPRKGSKTRQHQREVHFMKRLPRGHHTTEQSLRFEFLNYPPFTRSLRGSQRTWAPEPR

SEQ ID NO: 29 - DNA, 627 bp, Homo sapiens
atgtggaaatggatactgacacattgtgcctcagcctttccccacctgcccggctgctgctgctgctgctttttgttgctgttcttggtgtcttccgtccctgtcacctgccaagcccttggtcaggacatggtgtcaccagaggccaccaactcttcttcctcctccttctcctctccttccagcgcgggaaggcatgtgcggagctacaatcaccttcaaggagatgtccgctggagaaagctattctctttcaccaagtactttctcaagattgagaagaacgggaaggtcagcgggaccaagaaggagaactgcccgtacagcatcctggagataacatcagtagaaatcggagttgttgccgtcaaagccattaacagcaactattacttagccatgaacaagaaggggaaactctatggctcaaaagaatttaacaatgactgtaagctgaaggagaggatagaggaaaatggatacaatacctatgcatcatttaactggcagcataatgggaggcaaatgtatgtggcattgaatggaaaaggagctccaaggagaggacagaaaacacgaaggaaaaacacctctgctcactttcttccaatggtggtacactcatag

SEQ ID NO: 30 - protein, 208 aa, Homo sapiens
MWKWILTHCASAFPHLPGCCCCCFLLLFLVSSVPVTCQALGQDMVSPEATNSSSSSFSSPSSAGRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHS

SEQ ID NO: 31 - DNA, 4572 bp, Mus musculus
ggctttccaagggacttggaggtggagagaagggcccaacaaaacgccagccgccagccgccccccaaacaagaagtggctttcggaagacttcacatcaacaggcaccaccaaaaagagaaaggaaggagaagacaacagcgcctgggcagctgcctccagttctgacaactccaaagagacactttttaagtggccagcaggctgggactctgcagagaaggaccagaaggtgccaaccgcagaggggcgcagatgtcttcctgcacccccaccccacccactttgggttttgttcaccgtcctgtcatctgtttttcagacctcttttgcatctaacatggtgaagaaaggagtgaagaagagaacaaagtaacccccggggggagcgaagagctctggtgaccgacaccaccagttcctactgccgcggccacccacgtccactgttcaccctgagactggagagacgcaggcagcggatccgaggacggagcgaggacaggcagccggtccttcctagaagttatggatgttggtgcactcgcttctggccagatccgtacccagagggagctatccagaagccaccacctccagctgtctctctgcctcgcagcaggtcttacccttccagtatgttccttctgatgagacaatttccagtgccgagagtttcagtacaatgtggaaatggatactgacacattgtgcctcagcctttccccacctgccgggctgctgttgctgcttcttgttgctctttttggtgtcttcgttccctgtcacctgccaagctcttggtcaggacatggtgtcacaggaggccaccaactgctcttcttcctcctcgtccttctcctctccttccagtgcgggaaggcatgtgcggagctacaatcacctccaaggagatgtccgctggagaaggctgttctccttcaccaagtactttctcacgattgagaagaacggcaaggtcagcgggaccaagaatgaagactgtccgtacagtgtcctggagataacatcagtggaaatcggagttgttgccgtcaaagccatcaacagcaactattacttagccatgaacaagaaggggaaactctatggctcaaaagagtttaacaacgactgtaagctgaaagagagaatagaggaaaatggatacaacacctatgcatcttttaactggcagcacaatggcaggcaaatgtatgtggcattgaatggaaaaggagctcccaggagaggacaaaaaacaagaaggaaaaacacctctgctcacttcctccccatgacgatccaaacatagaagaaaacactgttggtggatgcagtacaaccaatgactctttggacagaaagagatggtatcctcactgaagactgtagctcaaaaggcaaagacatagccctgaattcagcttgtttaaaggaaggaaggctttggatgtttttgtactcactgctgacatacaaagttcttttcactagctctgtgtcattgtgtcatgccttataatcaagatagaggcaagtcaagtttggatggaagttatcctcaagtgaacaatgttgtggtgggggctgggctttttttgtttgtttgtttgtttcatttttaagtttttgtttttgaacttctgagatagaacttaaagaacatggaacactctgttgaatgatctttgggaaagttatttatggaatatgaacacatatcaaagactttcattgctcattcaagcctgatgattcaatgagcagtaagacacgcaagcatttactggaaagcacttgggtcatatcatatgcacaaccaaaggagctttgggtgtggcaccatggaagaattggatcagatttacaaatataaacatagtagtatgaaactgtcctaatacaaatagtatggtatgcttgtgcattctgtctccatccttttctatttccttctaagttatttatttaataggatgttaaatatcttttggggtttaaagagtatcttcaatgctgccctctggtttaccttttctctctctctctctctctctctctctctctctctctctctctctctctctctctctctctctctctctctctccctctctctccctccctcccccctctggcaccatacgcacattcatgacaaagtgttttaaaaccttggcaaacacttcagaaataggagatgagatcaaggaagcagtatgaatgccccatgcgctctcagttgacttaatttgcacttctgcaataaaaaacaccagcaatgactatggcagaattctgctatagattatgtaacagatatctgtcatcatttgtcaacatatatcagtccagagggacccttaccttaaaatgtagaaggccaaattctctttcattgtcttatttcatcttcaagaatatactaaaagaagaaaaaatgaattgttagactaacattgttgggttttttttttcctactgatgatggcttgccacaggtcacaatggcaaatgatgcaaaggttatctgcacatacatgagccctttgtaaggcccacagaatccttctccctcaaaagaaccaaaaaaggaaatttggtatgaagtgcaactctccctggggcttaacctgagcaaatatatcctagtatatgagtaaccatatactgacacctgttcaagctgaatggtctagtctttacagaaccacataaaccttgttttctgtaaatttaaaatgttctagaaggttccataatataaccacattgaaattcattttcttagaaaaggtatagaaagcagtatgtaagtgtgccatgcaccctcgctctgtagatcactaaataaacacgtaagccttatttgcagtgtctgtagtgattttaagaatgtaggaaacacttctaaaaaaattttaaaggataactctgagatgatattgatgctgcagtcttctttcttgtttggaaatgtctgtttattttcattgtttggattcagtattttgataggaacaaaaagactcaccaaatgtgtctgtttactaaaatttaacctctagagaggctagtgatttgtgatcctcttctaacttatttgtgctgatgcttgaccagtacaaatcagctttttaaaatattattattaaaggttgatcagtcattttaaaattggccttttttttcagaatgttcctacaggtcataatttatgatttctttgaaaagcttgcatttcaagagaaaagcacagaggcacaatgctttggtttatgggtataggttgcattttgtggtgttctttcaacttgttttctgacaaatgggatttttaaaatgtatacttcttgtggttggattctgtatgttagagtttaattggtaactgagtctaaaggctctaatgtaatgaatctctagaagaactaggtatctttttttacttttattttaaaataataattatacctgacacatgaccatggaccacccacaaccaaaattaaatgtttggggagacaaactatagtattcagtgacaagggtaacagcaaatagtgcagacgttggattcttatttcactttgccatttagattactaaagagactatgtgtaaacagtcatcattatagtactcaagacattaaacagcttctagcaaaatgtatcaaagcttgcagagtccaaaaatagaaaacatctttccccctctcccaccctacatttccccctgtatgcatcctaacagagataaatacaaaatgaattcggtaaggagagaggagattcttcttcacttcatatttgtttgatattaatagagaattctggtcctttttacaactactgaaagaaaagaagttcagtcctaaattttgtgtgttaaaaaaagaaaagattttgtgagttctgcctccgtgggaagtgtgggcactgctccaccatgctgaagtgtgttagccacgggtacagagcatatgactgttgacatcagactccttaaagatacagaatcgcttccctcctcctaatcctcaaaaggctgaacagtgtatattatgttacatttaaataaaggcaataaaaatgctgggaaaagagaataaaagtactgttcttattttatttcctttctttcttctcttctcttttcttttctttccttttcttttttttttcctttttttttcttttttttttttattagcctaaaactatacctggtaatgagatcagctccagggctgtgtgcatggcaggatgtggttaaatgccccacagccccaaacaacaacaacagaaaaaaaaattactcaaacatttgtaaggtttctttaatgttttacatgtgtgagccggctatccttaccctaataacaaccaaatgctttcgggttctcctaactactcaggtccacctagtttacacagtggataaagaagaatgaattgaaaacaaggatggcttgtgcaacaatgagaggctcttggaggaaagccaggagctgcaaacgttgacttccagggcatggaaaagaccaacgaatttgatttgaaaagt

SEQ ID NO: 32 - protein, 209 aa, Mus musculus
MWKWILTHCASAFPHLPGCCCCFLLLFLVSSFPVTCQALGQDMVSQEATNCSSSSSSFSSPSSAGRHVRSYNHLQGDVRWRRLFSFTKYFLTIEKNGKVSGTKNEDCPYSVLEITSVEIGVVAVKAINSNYYLAMNKKGKLYGSKEFNNDCKLKERIEENGYNTYASFNWQHNGRQMYVALNGKGAPRRGQKTRRKNTSAHFLPMTIQT

SEQ ID NO: 33 - DNA, 624 bp, Homo sapiens
atggcagaggtggggggcgtcttcgcctccttggactgggatctacacggcttctcctcgtctctggggaacgtgcccttagctgactccccaggtttcctgaacgagcgcctgggccaaatcgaggggaagctgcagcgtggctcacccacagacttcgcccacctgaaggggatcctgcggcgccgccagctctactgccgcaccggcttccacctggagatcttccccaacggcacggtgcacgggacccgccacgaccacagccgcttcggaatcctggagtttatcagcctggctgtggggctgatcagcatccggggagtggactctggcctgtacctaggaatgaatgagcgaggagaactctatgggtcgaagaaactcacacgtgaatgtgttttccgggaacagtttgaagaaaactggtacaacacctatgcctcaaccttgtacaaacattcggactcagagagacagtattacgtggccctgaacaaagatggctcaccccgggagggatacaggactaaacgacaccagaaattcactcactttttacccaggcctgtagatccttctaagttgccctccatgtccagagacctctttcactataggtaa

SEQ ID NO: 34 - protein, 207 aa, Homo sapiens
MAEVGGVFASLDWDLHGFSSSLGNVPLADSPGFLNERLGQIEGKLQRGSPTDFAHLKGILRRRQLYCRTGFHLEIFPNGTVHGTRHDHSRFGILEFISLAVGLISIRGVDSGLYLGMNERGELYGSKKLTRECVFREQFEENWYNTYASTLYKHSDSERQYYVALNKDGSPREGYRTKRHQKFTHFLPRPVDPSKLPSMSRDLFHYR

SEQ ID NO: 35 - DNA, 3040 bp, Mus musculus
ccgccaagacaggcagttcgcgccccggggccccggctcggcccgcgccgccgccggccccgccatggcggaggtcgggggcgtctttgcctccttggactgggacctgcacggcttctcctcctctctggggaacgtgcccttagctgactccccgggtttcctgaacgagcgcctgggccagatcgaggggaagctgcagcgcggctcgcccacagacttcgcccacctgaaggggatcctgcggcgccgccagctctactgccgcaccggcttccaccttgagatcttccccaacggcacggtgcacggcacccgccacgaccacagccgcttcggaattctggaatttatcagcttggctgtggggctgatcagcatcaggggagtggactctggcctgtacctaggaatgaatgagcgaggagagctctatggatcgaagaaactcacacgtgaatgtgttttccgggaacagtttgaagaaaactggtacaacacctatgcctccaccttgtacaaacactcggactcggagagacagtattatgtggccctgaataaagacggctcaccccgggagggatacaggactaaacgacaccagaaattcactcactttttaccaaggccagtagatccttctaagttgccctccatgtccagagatctcttccgctataggtaatggacctgtggtgccacctctgtgacccatgtactctgtgaccatggttggctctgcaaacactatactcatagcttttcaatccttccttcctatcaattagataacagagaagcactcactgttcaatgtgacccagctgtccccttgttcaaagtgtatatcaaggttgggttattttggggagggacatgggggaggggtggttctgagggaaatgtgtatatactttttgttttactcatgttctttcccgagatgtggcattacaaagaacaagggcccccattaagggccaaggctgtcttgcctcacaatctacccagataccttgaaatgggggtacaaatgaaagaccaaggaatctgccacagatgctacctaaagagctctgggaaagtgttatttggaaaatcctaggggctccaaccctgctgcaagccatccctaccctggctctgcagatctctcattcgtcctgacatgcagttttcctgccaccggaagtgggagatatggggtaggttgtaaggaaatgatattaaaatgtaagcgtggatactgtgcataaggacaaactatttataaaatgtatatgctatttattttatatatttatttatttcaaaataattttatttttaatgagagatgctatgactggattttcttcagaaagaataaggtcctactgaaacaggaaaaaatgagttattaaaggcttttatttggcaataaaattgtctttatattataaaaccagtgtctaattctattgctttacacattcagtagaatgaagaggctggccagcttcttttttttttcctccaggttaataaaatccccaatgacaggctgatgcagcttgagctgggtaactccatcacctagtttggtagcattatccaggcaccttcttcaagacctactgctcttttttggtcccatgactcaagggagctttaggaagacctcaactcacagctcctactgaagctcccatttgtaaaccttcaggattgggcatagaggaacctttcttggggacatgatgagggcccacagctctaagaacagcgacctaatcagtggatctttatttcagatatgaaggtgaagcaaattaaaatagtagtcagcaaggagaggagtttttgagccaatcaagttccctgctggaagacatcatcctactggctctgaccatccccagcatcttttaccagggcttctagaggattctgggaaactgttagcacattcccttagagcagctcttgcccagcagaatcacctggaaactgttgctcaaggatagaaatcagcagaaacttagttctgggccaggcactttgcatggtaccgagctttccagagtttccctaagcatgccacatcatacatgtgatccctaagcatatagtgatagatttttgttagtggttttttttttgttgtttttttttgtttttttttttttttgttttgttttttgcaggcaaatggctcataagtgaattggtgggcaagtgttagtccagagaatgaagaaggattagtttcatacctcagagctcttcctctccgagatcagccctaaagtatcttgacttcaaatccatcgactctggtatttccagaagtaggggtgcaagaggttggaatcaaagcaataaaagtcttggtgagcacaccaacagtatgaaggcctaattggcaaaggtttggaatctaggatagttcttcctcatcctgcccattatgaaggcatctgctacttttctaactaattgtacctgtgagtcagacagacaccagctcacatgcaaagcaaacccagtggtataggtgcagatcaatggtgaagtgcttcttagcatgggacgagccctagattccatccctccagcaccacaaaaaagcaaaaccaaaagagctattaaagttcacctcaatttaagtacccaataaagaagtcacacacatatggggatactgacctgtggtgagagctaaattgagggctgggactatagctcaatggaagaacactagctttccatggtccagtttgtagtgctgcaagaaataagtcagtaaataaagacaaaaactaacaaacaaaattaagttattacatggcatgtagtcattggctagcatgctgattgggggaatccctaaggtggacatttcagtgggctaggcttagtagggtgttttccaacagtgataatgtctgctttgaagaaccaatagcattaaggaaagcaaaacattgtttatgttgagctatggaaaagagacactgtctataaaattctctaaaattaaaaacctgctacatcc

SEQ ID NO: 36 - protein, 207 aa, Mus musculus
MAEVGGVFASLDWDLHGFSSSLGNVPLADSPGFLNERLGQIEGKLQRGSPTDFAHLKGILRRRQLYCRTGFHLEIFPNGTVHGTRHDHSRFGILEFISLAVGLISIRGVDSGLYLGMNERGELYGSKKLTRECVFREQFEENWYNTYASTLYKHSDSERQYYVALNKDGSPREGYRTKRHQKFTHFLPRPVDPSKLPSMSRDLFRYR

SEQ ID NO: 37 - DNA, 1238 bp, Homo sapiens
acctctccagcgatgggagccgcccgcctgctgcccaacctcactctgtgcttacagctgctgattctctgctgtcaaactcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgagaagcagaagcagttcgagtttgtgggctccgcccccacccgccggaccaagcgcacacggcggccccagcccctcacgtagtctgggaggcagggggcagcagcccctgggccgcctccccacccctttcccttcttaatccaaggactgggctggggtggcgggaggggagccagatccccgagggaggaccctgagggccgcgaagcatccgagcccccagctgggaaggggcaggccggtgccccaggggcggctggcacagtgcccccttcccggacgggtggcaggccctggagaggaactgagtgtcaccctgatctcaggccaccagcctctgccggcctcccagccgggctcctgaagcccgctgaaaggtcagcgactgaaggccttgcagacaaccgtctggaggtggctgtcctcaaaatctgcttctcggatctccctcagtctgcccccagcccccaaactcctcctggctagactgtaggaagggacttttgtttgtttgtttgtttcaggaaaaaagaaagggagagagaggaaaatagagggttgtccactcctcacattccacgacccaggcctgcaccccacccccaactcccagccccggaataaaaccattttcctgcaaaaaaaaaaaaaaaaaa

SEQ ID NO: 38 - protein, 216 aa, Homo sapiens
MGAARLLPNLTLCLQLLILCCQTQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFEFVGSAPTRRTKRTRRPQPLT

SEQ ID NO: 39 - DNA, 1687 bp, Mus musculus
cttaaggagatgaggttgccgcatcaaacctgagggcgggagctgctgaccctgaggaaggggagggcaaggcgcctccctccctgtgcttgccgctggccccgcacttcacggagaccggcaggttggtgcgggtcaggaataaatcaagagagatcgtaggggccagcctcctgatagaggacgctctcgcagctccaatcctttgcgcccggggacctctgcccgaagccagtagcccaagagagcgagtacgccaatcggaccagctccggcttcgcagccccactggcccggaaggtggacacctcgcacctcagtctctcaattacgcctccccttcattcccccctcggctagccttccccttccctccatctcctttcattcctgaaaacctgtggctccgagaaaccttggcttctctgggactctacctcaggggcctggccacatccgctcctgagtttggggagcagaggggcaatcgccccaagaagtctctccagcgatgggagccgcccgcctgctgcctaaccttaccctgtgcttgcagctattgattctctgctgtcaaacacagggggagaatcacccgtctcctaattttaaccagtacgtgagggaccagggcgctatgaccgaccagctgagcaggcggcaaatccgtgaataccagctctacagccggaccagtggcaagcacgtgcaggtcaccggacgtcgcatctctgccaccgcagaggatggcaacaagttcgccaagctcatcgtggagacagatacattcggcagcagagtccgcatcaagggggcagagagcgagaagtacatctgtatgaacaagaggggcaagctgattgggaagccgagcgggaagagcaaagactgcgtgttcaccgagatcgtactggagaacaactacacggccttccagaacgcccggcacgagggctggttcatggctttcactcggcagggccggccacgccaggcctcccggagccgccagaaccagcgagaggcccacttcatcaagcgcctctaccaaggccagctgccttttcccaaccacgctgaaaggcagaagcagttcgaatttgtgggctccgcccccactcgcaggaccaagcgcactcggaggccccagtcccaaacgtagtcagggaggccagtagtgtgccaggctgggtagatgcccctcagcagccttccctctcttctctaactcatccaaagatgggggctggggaggaaggtggggagccagtctccaaaaggaccccgagggcatgaagtttctgatccccactgggaagagacagtacttgtgtatcccagggtgggcaccattctatggaacaggtttgaggccagaggcagaccccggcagaagaaatagggtctccttgacctaaaatgcctgcttcccagcgtctacctgaaataagcgggctgctcagcagattccagagcttgcacaactgtgctcaaatcttgctccctgaacctctctgaatctgaaccaaatagccaaactcttggccagaccgtagggacttacggttttgttttcaaaaacagacagagacagaaagagaaacaaaagccagtcagccactgttcttatttctcgccccaggcctgcgtccccaccccaagccccagaataaaaccattttcctgc

SEQ ID NO: 40 - protein, 216 aa, Mus musculus
MGAARLLPNLTLCLQLLILCCQTQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAERQKQFEFVGSAPTRRTKRTRRPQSQT

SEQ ID NO: 41 - DNA, 1999 bp, Homo sapiens
cacggccggagagacgcggaggaggagacatgagccggcgggcgcccagacggagcggccgtgacgctttcgcgctgcagccgcgcgccccgaccccggagcgctgacccctggccccacgcagctccgcgcccgggccggagagcgcaactcggcttccagacccgccgcgcatgctgtccccggactgagccgggcagccagcctcccacggacgcccggacggccggccggccagcagtgagcgagcttccccgcaccggccaggcgcctcctgcacagcggctgccgccccgcagcccctgcgccagcccggagggcgcagcgctcgggaggagccgcgcggggcgctgatgccgcagggcgcgccgcggagcgccccggagcagcagagtctgcagcagcagcagccggcgaggagggagcagcagcagcggcggcggcggcggcggcggcggcggaggcgcccggtcccggccgcgcggagcggacatgtgcaggctgggctaggagccgccgcctccctcccgcccagcgatgtattcagcgccctccgcctgcacttgcctgtgtttacacttcctgctgctgtgcttccaggtacaggtgctggttgccgaggagaacgtggacttccgcatccacgtggagaaccagacgcgggctcgggacgatgtgagccgtaagcagctgcggctgtaccagctctacagccggaccagtgggaaacacatccaggtcctgggccgcaggatcagtgcccgcggcgaggatggggacaagtatgcccagctcctagtggagacagacaccttcggtagtcaagtccggatcaagggcaaggagacggaattctacctgtgcatgaaccgcaaaggcaagctcgtggggaagcccgatggcaccagcaaggagtgtgtgttcatcgagaaggttctggagaacaactacacggccctgatgtcggctaagtactccggctggtacgtgggcttcaccaagaaggggcggccgcggaagggccccaagacccgggagaaccagcaggacgtgcatttcatgaagcgctaccccaaggggcagccggagcttcagaagcccttcaagtacacgacggtgaccaagaggtcccgtcggatccggcccacacaccctgcctaggccaccccgccgcggcccctcaggtcgccctggccacactcacactcccagaaaactgcatcagaggaatatttttacatgaaaaataaggaagaagctctatttttgtacattgtgtttaaaagaagacaaaaactgaaccaaaactcttggggggaggggtgataaggattttattgttgacttgaaacccccgatgacaaaagactcacgcaaagggactgtagtcaacccacaggtgcttgtctctctctaggaacagacaactctaaactcgtccccagaggaggacttgaatgaggaaaccaacactttgagaaaccaaagtcctttttcccaaaggttctgaaaggaaaaaaaaaaaaaacaaaaaaaaagaaaaacaaagagaaagtagtactccgcccaccaacaaactccccctaactttcccaatcctctgttcctgccccaaactccaacaaaaatcgctctctggtttgcagtcatttatttattgtccgctgcaagctgccccgagacaccgcgcagggaaggcgtgcccctgggaattctccgcgcctcgacctcccgacgacagacgcctcgtccaatcatggtgaccctgccttgctcgcagttctggaggatgctgctatcgaccttccgtgactcacgtgacctagtacaccaatgataagggaatattttaaaaccagctatattatatatattatatatatataagctatttatttcacctctctgtatattgcagtttcatgaaccaagtattactgcctcaacaattaaaaacaacagacaaattatttaaaaaaccaaaaaaaaaaaaaaaaaa

SEQ ID NO: 42 - protein, 207 aa, Homo sapiens
MYSAPSACTCLCLHFLLLCFQVQVLVAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSRRIRPTHPA

SEQ ID NO: 43 - DNA, 1094 bp, Mus musculus
ggcacgagcggcaaggagggagcagcagcagcggcggcggcggcggcggcggcggcggaggcgcccggtcccggccgcgcggagcggacatgtgcaggctgggctaggagccgccgcctccccccccgccccgcgatgtattcagcgccctccgcctgcacttgcctgtgtttacactttctactgctgtgcttccaggttcaggtgttggcagccgaggagaatgtggacttccgcatccacgtggagaaccagacgcgggctcgagatgatgtgagtcggaagcagctgcgcttgtaccagctctatagcaggaccagtgggaagcacattcaagtcctgggccgtaggatcagtgcccgtggcgaggacggggacaagtatgcccagctcctagtggagacagataccttcgggagtcaagtccggatcaagggcaaggagacagaattctacctgtgtatgaaccgaaaaggcaagctcgtggggaagcctgatggtactagcaaggagtgcgtgttcattgagaaggttctggaaaacaactacacggccctgatgtctgccaagtactctggttggtatgtgggcttcaccaagaaggggcggcctcgcaagggtcccaagacccgcgagaaccagcaagatgtacacttcatgaagcgttaccccaagggacaggccgagctgcagaagcccttcaaatacaccacagtcaccaagcgatcccggcggatccgccccactcaccccggctaggtccggccacactcacccccccagagaactacatcagaggaatatttttacatgaaaaataaggaagaatctctatttttgtacattgtgtttaaaagaagacaaaaactgaacctaaagtcttgggaggaggggcgataggattccactgttgacctgaaccccatgacaaaggactcacacaaggggaccgctgtcaacccacaggtgcttgcctctctctaggaggtgacaattcaaaactcatccccagaggaggacttgaacgaggaaactgcgagaaaccaaagtcctttccccccaaaggttctgaaagcaaacaaaacaaaaacaaa

SEQ ID NO: 44 - protein, 207 aa, Mus musculus
MYSAPSACTCLCLHFLLLCFQVQVLAAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQAELQKPFKYTTVTKRSRRIRPTHPG

SEQ ID NO: 45 - DNA, 1016 bp, Homo sapiens
agcgacctcagaggagtaaccgggccttaactttttgcgctcgttttgctataatttttctctatccacctccatcccacccccacaacactctttactgggggggtcttttgtgttccggatctccccctccatggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttgaagtgcgatagtgacattatggaagagtcaaaccacaaccattctttcttgtcatagttcccatcataaaataatgacccaaggagacgttcaaaatattaaagtctattttctactgagagactggatttggaaagaatattgagaaaaaaaaccaaaaaaaattttgactagaaatagatcatgatcactctttatatgtggattaagttcccttagatacattggattagtccttaccagtagac

SEQ ID NO: 46 - protein, 211 aa, Homo sapiens
MAPLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 47 - DNA, 1476 bp, Mus musculus
tctagatgtttctgcaaggccagcagccagcatcctcctacccaaggagagaagatcaatctaagagggtgcactgcaccgtccccacagggacctcacaggagtaacccagccttaacttttttggtacctatttggctataatttttctgcattcgcctcgccacccttgctacactttttattggcggtgcagagggaagtctcctgttttctggaatttcagtcatcctgtccggcccgtccatggctcccttgaccgaagtcggggctttcttgggcggcctggagggcttgagccagcaggtggggtcgcacttcttgctgcctcctgcaggggagaggccaccgctgttaggggagcggcggggcgcgttggagaggggcgcccgcggcgggcccggttccgtggagctggcgcacctgcacggcatcctgcgccgccggcagctctactgccgcaccggcttccacctgcagatcctgcccgacggcaccgtgcagggcacccggcaggatcacagtctcttcggtatcctggaattcatcagtgtggcagtgggactggtcagtatcagaggtgtggacagtggcctgtaccttgggatgaatgacaaaggagaactttatggatcagagaaattgacttctgaatgcatcttcagggaacaatttgaagagaactggtataatacctattcgtccaacatatataaacatggagacacgggtcgcaggtattttgtagcacttaacaaggatggaactccaagagatggtgccaggtccaaaagacatcaaaagtttacccactttttaccaagaccagtagacccagaaagagttccagaattatacaaagacctactgatgtacacttgatgaatctagagccattgtttaaaaatcacagttcctgctgttaaataacaccgaagaagacgttcaggatattacgggagtctgcttttcactgaaagactctatttgggaagaaaattgagagtaaggaattaacttgaagcaaagcaagatcattctccgtaagtggattgtagttccttagacacgttgtttcagtcttaccagtagactgacgatgctgaaatcagttcatctgcggataatgtgaaccttgctgctgacgccgcatgtctctggataatgtttacttggacagttatcttaaaaatagatacttcatgttgaagaagtggattgagatgacataattactgcctcataaattctgaggaccttgtagaaaggttagaacgttatacataaaacaaaaatcaaaatactagatgactttgatctacaaaccacaccacactgagatgggacttttcttttagaagaatggacctatttatacttttttcaatttacaaatatttattatatatatttatttattaaagagtttatttttactagtatatgaagactatacaaaataaaacaaacaaaaag

SEQ ID NO: 48 - protein, 211 aa, Mus musculus
MAPLTEVGAFLGGLEGLSQQVGSHFLLPPAGERPPLLGERRGALERGARGGPGSVELAHLHGILRRRQLYCRTGFHLQILPDGTVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 49 - DNA, 513 bp, Homo sapiens
atgcgccgccgcctgtggctgggcctggcctggctgctgctggcgcgggcgccggacgccgcgggaaccccgagcgcgtcgcggggaccgcgcagctacccgcacctggagggcgacgtgcgctggcggcgcctcttctcctccactcacttcttcctgcgcgtggatcccggcggccgcgtgcagggcacccgctggcgccacggccaggacagcatcctggagatccgctctgtacacgtgggcgtcgtggtcatcaaagcagtgtcctcaggcttctacgtggccatgaaccgccggggccgcctctacgggtcgcgactctacaccgtggactgcaggttccgggagcgcatcgaagagaacggccacaacacctacgcctcacagcgctggcgccgccgcggccagcccatgttcctggcgctggacaggaggggggggccccggccaggcggccggacgcggcggtaccacctgtccgcccacttcctgcccgtcctggtctcctga

SEQ ID NO: 50 - protein, 170 aa, Homo sapiens
MRRRLWLGLAWLLLARAPDAAGTPSASRGPRSYPHLEGDVRWRRLFSSTHFFLRVDPGGRVQGTRWRHGQDSILEIRSVHVGVVVIKAVSSGFYVAMNRRGRLYGSRLYTVDCRFRERIEENGHNTYASQRWRRRGQPMFLALDRRGGPRPGGRTRRYHLSAHFLPVLVS

SEQ ID NO: 51 - DNA, 489 bp, Mus musculus
atgcgcagccgcctctggctgggcctagcctggctgctgttggcgcgggcaccgggcgctccgggagggtacccgcatctggagggcgacgtgcgctggcgccgcctcttctcctccactcactttttcctgcgtgtggaccttggtggtcgggtgcaggggacgcgttggcggcacggccaggacagtatagtggagatccgttctgtccgtgtgggcactgtggtgatcaaagctgtgtactcaggcttctatgtggccatgaatcgcaggggccgcctctatgggtcgcgggtctactctgtggactgtaggttccgggagcgcatcgaggagaacggctacaacacatacgcctcgcgacgttggaggcaccgcggccgacccatgttcctggcacttgacagccaaggcattcccaggcaaggcagacggacacgacggcaccaactgtccacacacttcctgccagtcttggtctcgtcttga

SEQ ID NO: 52 - protein, 162 aa, Mus musculus
MRSRLWLGLAWLLLARAPGAPGGYPHLEGDVRWRRLFSSTHFFLRVDLGGRVQGTRWRHGQDSIVEIRSVRVGTVVIKAVYSGFYVAMNRRGRLYGSRVYSVDCRFRERIEENGYNTYASRRWRHRGRPMFLALDSQGIPRQGRRTRRHQLSTHFLPVLVSS

SEQ ID NO: 53 - DNA, 940 bp, Homo sapiens
ctgtcagctgaggatccagccgaaagaggagccaggcactcaggccacctgagtctactcacctggacaactggaatctggcaccaattctaaaccactcagcttctccgagctcacaccccggagatcacctgaggacccgagccattgatggactcggacgagaccgggttcgagcactcaggactgtgggtttctgtgctggctggtcttctgctgggagcctgccaggcacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcctgaagccagaggctgtttactatgacatctcctctttatttattaggttatttatcttatttatttttttatttttcttacttgagataataaagagttccagaggagaaaaaaaaaaaaaaaaaaaaaaaaaaaaaaaaaaaaaaaaaaaaaaaaaaaaaaa

SEQ ID NO: 54 - protein, 209 aa, Homo sapiens
MDSDETGFEHSGLWVSVLAGLLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 55 - DNA, 947 bp, Mus musculus
agacagccttagtgtcttctcagctggggattcaacacaggagaaacagccattcactttgcctgagccccagtctgaacctgacccatccctgctgggcaccggagtcagaacacaattccagctgccttggctcctcagccgctcgcttgccaggggctctcccgaacggagcgcagccctgatggaatggatgagatctagagttgggaccctgggactgtgggtccgactgctgctggctgtcttcctgctgggggtctaccaagcataccccatccctgactccagccccctcctccagtttgggggtcaagtccggcagaggtacctctacacagatgacgaccaagacactgaagcccacctggagatcagggaggatggaacagtggtaggcgcagcacaccgcagtccagaaagtctcctggagctcaaagccttgaagccaggggtcattcaaatcctgggtgtcaaagcctctaggtttctttgccaacagccagatggagctctctatggatcgcctcactttgatcctgaggcctgcagcttcagagaactgctgctggaggacggttacaatgtgtaccagtctgaagcccatggcctgcccctgcgtctgcctcagaaggactccccaaaccaggatgcaacatcctggggacctgtgcgcttcctgcccatgccaggcctgctccacgagccccaagaccaagcaggattcctgcccccagagcccccagatgtgggctcctctgaccccctgagcatggtagagcctttacagggccgaagccccagctatgcgtcctgactcttcctgaatctagggctgtttctttttgggtttccacttatttattacgggtatttatcttatttatttattttagtttttttttcttacttggaataataaagagtctgaaagaaaaatgtgtgtt

SEQ ID NO: 56 - protein, 210 aa, Mus musculus
MEWMRSRVGTLGLWVRLLLAVFLLGVYQAYPIPDSSPLLQFGGQVRQRYLYTDDDQDTEAHLEIREDGTVVGAAHRSPESLLELKALKPGVIQILGVKASRFLCQQPDGALYGSPHFDPEACSFRELLLEDGYNVYQSEAHGLPLRLPQKDSPNQDATSWGPVRFLPMPGLLHEPQDQAGFLPPEPPDVGSSDPLSMVEPLQGRSPSYAS